(19) United States
(12) Patent Application Publication
(10) Pub. No.: US 2025/0134895 A1
(43) Pub. Date: May 1, 2025

(54) METHODS AND COMPOSITIONS FOR TREATING ALPHA-SYNUCLEIN-MEDIATED NEURODEGENERATION

(71) Applicant: UNIVERSITY HEALTH NETWORK, Toronto (CA)

(72) Inventors: Lorraine V. Kalia, Toronto (CA); Suneil K. Kalia, Toronto (ON)

(21) Appl. No.: 18/684,053

(22) PCT Filed: Aug. 16, 2022
(86) PCT No.: PCT/CA2022/051242

§ 371 Date: Feb. 15, 2024

Related U.S. Application Data
(60) Provisional application No. 63/233,619, filed on Aug. 16, 2021.

(51) Int. Cl. A61K 31/52; A61K 31/4178; A61K 31/436; A61K 31/438; A61K 31/513; A61P 25/28

(52) U.S. Cl. CPC A61K 31/52; A61K 31/4178; A61K 31/436; A61K 31/438; A61K 31/513; A61P 25/28

(57) ABSTRACT

Provided is a method of treating a subject with an α-synucleinopathy neurodegenerative disorder, the method comprising administering one or more therapeutic(s) to the subject, a method of treating a subject with a high risk of developing an α-synucleinopathy neurodegenerative disorder, the method comprising administering one or more therapeutic(s) to the subject, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof.

### RELATED APPLICATIONS

This Patent Cooperation Treaty application claims the benefit of priority of U.S. Provisional Application 63/233,619 filed Aug. 16, 2021, which is incorporated by reference herein in its entirety.

### FIELD

The present disclosure relates to therapeutics that may be repurposed as a disease-modifying therapy for Parkinson's disease (PD) and other α-synucleinopathy neurodegenerative disorders.

### BACKGROUND

Parkinson's disease (PD) is a common and disabling neurodegenerative disorder characterized by dopaminergic neuron loss induced by α-synuclein oligomers resulting in motor impairment. There is an urgent need for disease-modifying therapies for PD, but drug discovery is challenged by a lack of in vivo models that recapitulate early stages of neurodegeneration, when treatments are expected to have their greatest impact. Further, rodents and invertebrate organisms, such as the nematode worm Caenorhabditis elegans, provide in vivo models of human disease processes that can be instrumental for initial studies of potential pharmacological treatments.

PD is the most common neurodegenerative movement disorder, and its prevalence is projected to double over the next two decades.¹ Hence, there is an urgent need for the discovery of disease-modifying therapies that will slow the neurodegenerative process and thereby reduce disease progression and associated morbidity. PD is defined by motor impairment due to selective dysfunction and prominent loss of dopaminergic neurons in the substantia nigra pars compacta (SN). Abnormal accumulation of the protein α-synuclein and its aggregation into oligomers and fibril-containing inclusions, termed Lewy bodies, are also defining features. Missense mutations in SNCA, the gene encoding α-synuclein, promote aggregation of mutated α-synuclein and cause inherited monogenic forms of PD (e.g., A30P or A53 T mutation). Furthermore, multiplications of SNCA or small nucleotide polymorphisms in SNCA that increase expression of wild-type α-synuclein are also associated with PD, providing strong evidence that α-synuclein can mediate neurodegeneration in both its mutant and wild-type forms.² Thus, α-synuclein has emerged as a promising therapeutic target for disease modification in PD.³

Heat shock protein 90 (HSP90) and HSP70 have been found to co-localize with α-synuclein aggregates in PD patients (Daturpalli et al., 2013; Cox et al., 2018). HSP70 has been found to prevent α-syn oligomer and fibrillar aggregation in vitro (Gao et al., 2015) and in vivo (Moloney et al., 2014; Klucken et al., 2004). To date, human trials aimed at discovering disease-modifying therapies for PD have been unsuccessful.⁴ An important lesson from these failed attempts is that therapies may need to be initiated early in the neurodegenerative process, when interventions are expected to have their greatest impact.⁵ Yet, ongoing preclinical efforts still rely on animal models of later disease stages, often when over 50% of dopaminergic neurons are already lost. Hence, there is a need for in vivo models that recapitulate early stages of neurodegeneration for PD drug discovery. Invertebrate organisms, such as the nematode worm Caenorhabditis elegans, are instrumental for in vivo modelling of human disease processes to test potential pharmacological treatments.⁶ C. elegans is particularly amenable to modelling aspects of neurological diseases, such as PD, because the animal has a well-characterized nervous system, which uses many of the same neurotransmitters found in humans (e.g., dopamine) and mediates a diversity of behaviours, most involving motor function.

Transgenic C. elegans strains expressing α-synuclein have been developed but none provide a sensitive indicator of the earlier stages of dopaminergic neuron degeneration.⁷ In one of the commonly used models, wild-type α-synuclein fused to a fluorescent protein is expressed only within body wall muscle cells where large intramuscular protein inclusions of α-synuclein form spontaneously.⁸ While this model can be useful to examine α-synuclein aggregation, it has limited relevance to PD since intramuscular α-synuclein inclusions do not occur in the disease. Another commonly used transgenic C. elegans co-expresses wild-type or mutant α-synuclein with a fluorescent protein in dopaminergic neurons. Neurodegeneration is observed by changes in morphology of the fluorescent neurons, including shortened neuritic processes and rounding of soma, as well as overt neuron loss.^{9,10} However, these structural changes reflect severely compromised neurons and represent dopaminergic neuron degeneration at advanced stages.

Post-mortem autopsy studies have demonstrated that loss of dopaminergic neuron terminals is a key feature of earlier stages of PD.⁵ It is at the earlier disease stages, when dopaminergic neuron cell bodies are intact and terminals are not completely degenerated, that treatment with disease-modifying therapies is expected to be most effective in PD. Yet, most models used for drug discovery do not have measurable and modifiable markers that reflect early α-synuclein-mediated neurodegeneration.

Currently, targeting α-synuclein as a disease-modifying strategy for PD is an active area of drug development with small molecules, antibodies, and antisense oligonucleotides (ASOs) designed to reduce α-synuclein nearing or in human clinical trials.³

Drug repurposing in PD has had previous success, most notably in the repurposing of antiviral amantadine for the treatment of parkinsonian symptoms, which was approved in 1976 (nisar et al., 2019). However, more recent studies involving drug repurposing have had limited success. The investigation of caffeine for symptomatic treatment of PD reached clinical trial stage in 2012, following large-scale cohort studies in which caffeine consumption was correlated with a lower risk of developing PD (sääksjärvi et al, 2008; palacios et al., 2012). However, the clinical trial study failed to meet clinical primary endpoints, with no significant motor benefit in patients, suggesting the lower risk of PD cannot be explained by caffeine treatment in patients (postuma et al., 2017). Similarly, the calcium-channel blocker israpidine, currently approved for the treatment of hypertension, was investigated for the potential protection from calcium-channel associated depletion of dopaminergic neurons in PD (simuni et al., 2020). However, this trial also failed to exhibit clinical end-points, which may have been due to reduced brain bioavailability of the drug in humans compared to pre-clinical rodent models (simuni et al., 2020). Nilotinib, an approved therapy for chronic myeloid leukaemia, previously underwent clinical investigation in PD patients to potentially slow disease progression through its action as a protein tyrosine kinase inhibitor (simuni et al., 2021). However, recent clinical data revealed nilotinib to exert no significant changes on dopaminergic biomarker expression in patients (simuni et al., 2021). Most recently, diabetes drug exenatide, which acts as a glp-1 receptor agonist, has shown some potential for repurposing following evidence of pre-clinical protection against dopaminergic neuron terminal loss in the striatum in the mitopark mitochondrial mouse model of PD (wang et al,. 2021).

There are undoubtably many benefits to the utilization of drug repurposing in drug discovery. Starting with a compound that already has pre-existing human pharmacokinetic and toxicological data allows drugs to enter at Phase II, allowing significantly quicker clinical approval, and giving companies an exclusive market advantage through bypassing the lengthy and expensive pre-clinical and safety testing prior to approval. Traditional drug discovery takes around 13-15 years and >$1 billion to develop and bring a new drug to market (Chen et al., 2018), and <6% of the new molecules that enter Phase 1 clinical trials are approved by the FDA (Sun et al., 2016). The advantages of drug repurposing over traditional drug discovery have contributed to the success rate of drug repurposing at around 30% (Pillaiyar et al., 2020).

Rapamycin is a drug that is an immunosuppressant and has previously demonstrated efficacy in an in vivo α-synuclein toxicity model of PD.⁴⁵

Losartan is a drug which is used to treat high blood pressure. It has previously shown neuroprotective potential in vitro and in a mouse model, but not via an α-synuclein-mediated mechanism.^{82,83} Rifabutin is used for treatment of tuberculosis.

Abacavir is an antiviral which acts as a nucleoside analog reverse-transcriptase inhibitor (NRTI) and was approved for the treatment of human immunodeficiency virus (HIV)/AIDS in 1998 (Saag et al., 1998). Drug repurposing has previously been applied to investigate abacavir for treatment of Amyloid Lateral Sclerosis (ALS), in which it has shown successful clinical outcomes in safety and tolerability in ALS patients (Gold et al., 2019). In addition, abacavir is known to have high CNS penetration, with the ability to cross both the blood-brain and blood-CSF barriers and has been shown to enter both the brain and cisternal CSF within therapeutic concentrations, as demonstrated both in rodent models (Thomas et al., 2001) and humans (Letendre et al., 2008). Mass spectrometry has shown abacavir to have high penetration in the neocortex, thalamus and striatum of rats, penetration which was much higher in comparison to other antiviral NRTIs tested including stavudine and didanosine (Mdanda et al., 2020).

In vivo models that provide a sensitive indicator of the earlier stages of dopaminergic neuron degeneration, and disease-modifying drugs for PD are desirable.

### SUMMARY

Provided herein is the first identification of a C. elegans locomotor abnormality due to dopaminergic neuron dysfunction that models early α-synuclein-mediated neurodegeneration. The presently described approach of applying this in vivo model to a multi-step drug repurposing screen with artificial intelligence (AI)-driven in silico and in vitro methods resulted in the identification of therapeutics for example rifabutin, losartan, and abacavir as candidates for repurposing as a disease-modifying therapy for Parkinson's disease (PD) and other α-synucleinopathies. AI predictions that rifabutin, losartan, and abacavir reduce α-synuclein oligomers were validated in primary cortical rat neurons. Rifabutin, losartan, and abacavir were shown to reduce α-synuclein-mediated dopamine neuron degeneration in a C. elegans model and/or preclinical rat model of PD. Thus, rifabutin, losartan, and abacavir were identified as candidates for repurposing as disease modifying therapies for PD and other α-synucleinopathies.

An aspect of the disclosure includes a method of treating a subject with an α-synucleinopathy neurodegenerative disorder the method comprising administering one or more therapeutic(s) to the subject, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan or a combination thereof.

In some embodiments, the one or more therapeutic(s) is or comprises rifabutin, losartan, lamivudine, and/or abacavir. In some embodiments, the one or more therapeutic(s) is or comprises rifabutin, losartan, and/or abacavir.

Another aspect of the disclosure includes a method of treating a subject with a high risk of developing an α-synucleinopathy neurodegenerative disorder, the method comprising administering one or more therapeutic(s) to the subject, wherein the one or more therapeutic(s) is or comprise of rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof.

In some embodiments, the one or more therapeutic(s) is or comprises rifabutin, losartan, lamivudine, and/or abacavir. In some embodiments, the one or more therapeutic(s) is or comprises rifabutin, losartan, and/or abacavir.

Another aspect of the disclosure includes a method of inhibiting α-synuclein aggregation, the method comprising contacting a cell with one or more therapeutic(s), wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan or a combination thereof. For example, contacting includes administering to a subject, when the cell is in a subject. As another example α-synuclein aggregation can include intracellular and/or extracellular aggregation.

In some embodiments, the one or more therapeutic(s) is or comprises rifabutin, losartan, lamivudine, and/or abacavir. In some embodiments, the one or more therapeutic(s) is or comprises rifabutin, losartan, and/or abacavir.

Another aspect of the disclosure includes use of one or more therapeutic(s) for treating a subject with an α-synucleinopathy neurodegenerative disorder, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof.

In some embodiments, the one or more therapeutic(s) is or comprises rifabutin, losartan, lamivudine, and/or abacavir. In some embodiments, the one or more therapeutic(s) is or comprises rifabutin, losartan, and/or abacavir.

Another aspect of the disclosure includes use of one or more therapeutic(s) for treating a subject with a high risk of developing α-synucleinopathy neurodegenerative disorder, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof.

In some embodiments, the one or more therapeutic(s) is or comprises rifabutin, losartan, lamivudine, and/or abacavir. In some embodiments, the one or more therapeutic(s) is or comprises rifabutin, losartan, and/or abacavir.

Another aspect of the disclosure includes use of one or more therapeutic(s) in the manufacture of a medicament for the treatment of an α-synucleinopathy neurodegenerative disorder, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof.

In some embodiments, the one or more therapeutic(s) is or comprises rifabutin, losartan, lamivudine, and/or abacavir. In some embodiments, the one or more therapeutic(s) is or comprises rifabutin, losartan, and/or abacavir.

Another aspect of the disclosure includes use of one or more therapeutic(s) for inhibiting α-synuclein aggregation, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof.

In some embodiments, the one or more therapeutic(s) is or comprises rifabutin, losartan, lamivudine, and/or abacavir. In some embodiments, the one or more therapeutic(s) is or comprises rifabutin, losartan, and/or abacavir.

Another aspect of the disclosure includes one or more therapeutic(s) for inhibiting α-synuclein aggregation, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof.

In some embodiments, the one or more therapeutic(s) is or comprises rifabutin, losartan, lamivudine, and/or abacavir. In some embodiments, the one or more therapeutic(s) is or comprises rifabutin, losartan, and/or abacavir.

Another aspect of the disclosure includes one or more therapeutic(s) for treating an α-synucleinopathy neurodegenerative disorder, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof.

In some embodiments, the one or more therapeutic(s) is or comprises rifabutin, losartan, lamivudine, and/or abacavir. In some embodiments, the one or more therapeutic(s) is or comprises rifabutin, losartan, and/or abacavir.

An aspect of the disclosure comprises a method of treating a subject with an α-synucleinopathy neurodegenerative disorder the method comprising administering one or more therapeutic(s) selected from a group comprising rifabutin, losartan, and abacavir. In some embodiments, the subject is a mammal. In some embodiments, the subject is a human. In some embodiments, the α-synucleinopathy neurodegenerative disorder is selected from a group comprising Parkinson's Disease, dementia with Lewy bodies, multiple system atrophy, and Alzheimer's disease. In some embodiments, the one or more therapeutic(s) are rifabutin, losartan, and abacavir. In some embodiments, the therapeutic is rifabutin. In some embodiments, therapeutic is losartan. In some embodiments, the therapeutic is abacavir. In some embodiments, the method further comprises administering rapamycin. In some embodiments, the subject is administered the one or more therapeutics immediately following a clinical diagnosis of Parkinson's Disease, dementia with Lewy bodies, multiple system atrophy, Alzheimer's disease.

Another aspect of the disclosure includes a method of treating a subject with a high risk of developing an α-synucleinopathy neurodegenerative disorder, the method comprising administering one or more therapeutic(s) selected from the group comprising rifabutin, losartan, and abacavir. In some embodiments, the subject has known genetic risk factor(s) for developing α-synucleinopathy neurodegenerative disorder. In some embodiments, the α-synucleinopathy neurodegenerative disorder is selected from a group comprising Parkinson's Disease, dementia with Lewy bodies, multiple system atrophy, and Alzheimer's disease. In some embodiments, the subject has prodromal Parkinson's Disease, prodromal dementia with Lewy bodies, prodromal multiple system atrophy, and prodromal Alzheimer's disease (including those with idiopathic/isolated REM sleep behaviour disorder). In some embodiments, the subject is administered the one or more therapeutic(s) prior to clinical diagnosis of an α-synucleinopathy neurodegenerative disorder. In some embodiments, the subject is a mammal. In some embodiments, the subject is a human. In some embodiments, the therapeutics are rifabutin, losartan, and abacavir. In some embodiments, the therapeutic is rifabutin. In some embodiments, the therapeutic is losartan. In some embodiments, the therapeutic is abacavir. In some embodiments, the method further comprises administering rapamycin.

Another aspect of the disclosure includes a method of inhibiting α-synuclein aggregation, the method comprising contacting a cell with one or more therapeutic(s) selected from a group comprising rifabutin, losartan, and abacavir. In some embodiments, the cell is a mammalian cell. In some embodiments, the cell is a human cell. In some embodiments, the cell is a neuron. In some embodiments, the cell is in a subject afflicted with or at risk of developing an α-synucleinopathy neurodegenerative disorder. In some embodiments, the α-synucleinopathy neurodegenerative disorder selected from Parkinson's Disease, dementia with Lewy bodies, multiple system atrophy, or Alzheimer's disease. In some embodiments, the subject has prodromal Parkinson's Disease, prodromal dementia with Lewy bodies, prodromal multiple system atrophy, and prodromal Alzheimer's disease (including those with idiopathic/isolated REM sleep behaviour disorder). In some embodiments, the subject is administered the one or more therapeutic(s) prior to clinical diagnosis of an α-synucleinopathy neurodegenerative disorder. In some embodiments, the one or more therapeutic(s) are rifabutin, losartan, and abacavir. In some embodiments, the therapeutic is rifabutin. In some embodiments, the therapeutic is losartan. In some embodiments, the therapeutic is abacavir. In some embodiments, the method further comprises administering rapamycin. For example, contacting includes administering to a subject, when the cell is in a subject. As another example α-synuclein aggregation can include intracellular and/or extracellular aggregation.

Another aspect of the disclosure includes use of one or more therapeutic(s) selected from the group comprising rifabutin, losartan, and abacavir for treating a subject with an α-synucleinopathy neurodegenerative disorder.

Another aspect of the disclosure includes use of one or more therapeutic(s) selected from the group comprising rifabutin, losartan, and abacavir for treating a subject with a high risk of developing an α-synucleinopathy neurodegenerative disorder.

Another aspect of the disclosure includes use of one or more therapeutic(s) selected from the group comprising rifabutin, losartan, and abacavir in the manufacture of a medicament for the treatment of an α-synucleinopathy neurodegenerative disorder.

Another aspect of the disclosure includes use of one or more therapeutic(s) selected from the group comprising rifabutin, losartan, and abacavir for inhibiting α-synuclein aggregation.

Another aspect of the disclosure includes one or more therapeutic(s) selected from the group comprising rifabutin, losartan, and abacavir for inhibiting α-synuclein aggregation.

Another aspect of the disclosure includes one or more therapeutic(s) selected from the group comprising rifabutin, losartan, and abacavir for treating an α-synucleinopathy neurodegenerative disorder.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIGS. 1A-1F. Coiling is an early motor impairment of C. elegans that correlates with α-synuclein protein levels. (a) Top: Circularity values per frame from recordings of individual non-transgenic C. elegans (N2 wild-type strain) or transgenic C. elegans expressing A30P α-synuclein in dopaminergic neurons (syn). Bottom: Processed images (numbered 1 to 4) of the animal corresponding to different circularity values. Normal sinusoidal body shape corresponded to circularity values of ˜0.2 (left, image 2). Body shape during an omega turn corresponded to circularity values of ˜0.5 (left, images 1 and 3; right, image 4). Coiling animals had circularity values ranging from ˜0.6 to almost 1 (left, image 4; right, images 1 and 2). (b) Percentage of frames with a circularity value >0.6 for individual N2 (N=40 animals), syn (N=40 animals), or animals that do not express α-synuclein but have the same genetic background (tzls3) as syn animals (No syn) (N=41 animals) (one-way ANOVA with Tukey's post-hoc test, *** p<0.001). Each data point represents an individual animal. (c) Recordings were performed of N2, syn, or control animals expressing GFP instead of α-synuclein in dopaminergic neurons (N≥10 populations of n=10 animals per strain), and circularity values were calculated for each animal in each frame. Range of circularity values was divided into bins of 0.05 (20 bins from 0 to 1.0). Number of circularity values within each bin were counted and percentage relative to all counts was calculated (presented as log₁₀ of percentage). Circularity value of 0.6 (dotted line) was selected as the threshold to isolate the coiler phenotype. (d) Coiler scores of syn animals (N=3 populations of n=10 animals) and known coiler mutant strains: unc-10 (e102) (N=9 populations of n=10 animals), unc-69 (e587) (N=9 populations of n=10 animals), and unc-17 (e245) (N=9 populations of n=10 animals), normalized to control GFP animals (one-way ANOVA with Tukey's post-hoc test, ** p<0.01, **** p<0.0001 relative to syn animals). Each data point represents an individual population. (e) Coiler scores of animals expressing wild-type (WT), mutant A30P, or mutant A53 T α-synuclein in dopaminergic neurons. Control C. elegans express GFP only in dopaminergic neurons (GFP) or do not express either α-synuclein or GFP (No syn). Coiler scores are normalized to control GFP animals (N=10 populations of n=10 animals for each strain; each data point represents an individual population) (one-way ANOVA with Tukey's post-hoc test, *** p<0.001, **** p<0.0001 relative to GFP). (f) Correlation of coiler score and α-synuclein protein levels for 12 different C. elegans strains expressing α-synuclein in dopaminergic neurons (Pearson correlation, r=0.58, p<0.05). Each point of the scatter plot represents one animal strain. The syn protein: total protein ratio for the 3 lowest expressing strains ranged from 0.03 to 0.2×10⁻⁶.

FIGS. 2A-2E. Coiling results from early dopaminergic neuron dysfunction due to α-synuclein preceding neuronal loss. (a) Left: Loss of dopaminergic anterior deirid neuron (ADE) neurons over time for C. elegans expressing A30P α-synuclein plus GFP in dopaminergic neurons (N=3 populations of n=50 animals) and for C. elegans expressing GFP alone (N=4 populations of n=50 animals) (two-way ANOVA with Bonferroni's post-hoc test, ** p<0.01, *** p<0.001, **** p<0.0001 comparing syn plus GFP and GFP alone animals on the same experimental day). Each data point represents an individual population. Right: Representative fluorescent microscopy images of surviving ADE dopaminergic neurons (arrowheads). (b) CRE-GFP reporter in sublateral interneuron (SIA) cholinergic neurons is used to assess for intact (GFP expression “OFF”) or disrupted (GFP expression “ON”) presynaptic signalling from the upstream cephalic neuron (CEP) dopaminergic neuron. (c) Top: Representative fluorescent microscopy images of GFP positive SIA cholinergic neurons (arrowheads). Bottom: Quantification of number of GFP positive SIA neurons (left graph) and percentage of animals with GFP positive SIA neurons (right graph) for control animals not expressing α-synuclein (No syn) (N=104 animals), syn animals (N=100 animals), and a tyrosine hydroxylase (TH) mutant strain cat-2 (e1112) (N=104 animals) (one-way ANOVA with Tukey's post-hoc test, **** p<0.0001). (d) Left: Coiler scores of syn animals treated with increasing concentrations of exogenous dopamine, normalized to vehicle treated control GFP animals (N=3 populations (represented by each data point) of n=10 animals per treatment condition) (one-way ANOVA with Dunnett's post-hoc test, *p<0.05, ** p<0.01 relative to [Dopamine] 0 mM). Right: Coiler scores of control GFP animals treated with 1 mM raclopride (N=16 populations of n=10 animals) or vehicle (N=12 populations of n=10 animals), normalized to vehicle treated control GFP animals (two-tailed t-test, ** p<0.01). Each data point represents an individual population. (e) Gene ontology enrichment analysis of biological processes implicated in coiling using 97 genes from C. elegans strains previously reported to demonstrate coiling behaviour. Biological processes over-represented by a factor of ≥100 were ranked according to the −log₁₀ false discovery rate.

FIGS. 3A-3C. Alterations in protein control pathways that regulate α-synuclein accumulation affect coiling. (a) Left: Immunoblot of immunoprecipitated α-synuclein from syn C. elegans lysates. No α-synuclein was immunoprecipitated from GFP C. elegans lysates. The asterisk indicates the light chain of the immunoprecipitating antibody. Proteins co-immunoprecipitated with the anti-α-synuclein antibody were identified by mass spectrometry. Right: Proteins that were specifically co-immunoprecipitated from syn C. elegans lysates were analysed using gene ontology enrichment for biological process. Over-representation of proteins <0.05 FDR was ranked according to fold enrichment. (b) Left: Coiler scores of syn animals treated with 50 μM 17-(allylamino)-17-demethoxygeldanamycin (17-AAG) (N=21 populations of n=10 animals) or vehicle (N=17 populations of n=10 animals), normalized to vehicle treated control GFP animals (two-tailed t-test, *p<0.05). Right: Representative immunoblot for α-synuclein protein in lysates of treated animals (α-tubulin used as a loading control). (c) Left: Coiler scores of syn animals crossed with the mutant strain chn-1(by155). CHN-1 is the homolog of the human the co-chaperone protein, carboxyl-terminus of HSP70 interacting protein (CHIP). Coiler scores are normalized to control GFP animals (N=10 populations of n=10 animals for each strain) (one-way ANOVA with Tukey's multiple comparisons test, *p<0.05, **** p<0.0001). Right: Representative immunoblot for α-synuclein protein in lysates of syn animals and syn animals crossed with chn-1(by155) (α-tubulin used as a loading control). Each data point represents an individual population.

FIG. 4. Screening strategy to identify compounds that inhibit α-synuclein oligomers and α-synuclein-mediated cytotoxicity. An AI-driven in silico screen was first performed with IBM Watson for Drug Discovery Predictive Analytics to predict compounds with a high likelihood of inhibiting aggregation of α-synuclein into oligomers. Next, highly ranked drugs were tested for their ability to reduce α-synuclein oligomer levels in vitro using a Gaussia princeps luciferase protein-fragment complementation cell assay. Positive hits were then assessed in vivo by measuring their effects on the motor impairment of C. elegans expressing α-synuclein. Last, screen hits were validated in mammalian in vitro and in vivo models. Specifically, drugs that reduced the coiling of C. elegans were examined for their ability to reduce α-synuclein oligomers measured by YFP protein-fragment complementation in rat primary cortical neurons, and the top candidate was tested in an adeno-associated viral vectors (AAV)-based rat model of α-synuclein-mediated dopaminergic neurodegeneration.

FIGS. 5A-5C. Acetaminophen, caffeine, losartan, mercaptopurine, rapamycin, and rifabutin reduce α-synuclein oligomers in vitro. (a) Testing of 40 highly ranked compounds (1 μM or 10 μM) for their ability to reduce α-synuclein oligomer levels in vitro using a Gaussia luciferase protein-fragment complementation cell assay. Rapamycin (2 μM) and bafilomycin A1 (50 nM) were used as active controls (N=4 for each treatment) (one-tailed t-test, * p<0.05). (b) Etoposide, piroxicam, theophylline, and vincristine demonstrated reductions in viability at multiple concentrations, measured using PrestoBlue-mediated fluorescence and normalized to vehicle alone (N=3 with n=2−4 replicates for each concentration) (one-way ANOVA with Dunnett's post-hoc test, * p<0.05, ** p<0.01, *** p<0.001, **** p<0.0001). No statistically significant changes in viability were observed with acetaminophen, caffeine, losartan, mercaptopurine, rapamycin, or rifabutin. (c) No statistically significant changes in the activity of full-length Gaussia luciferase alone were found with acetaminophen, caffeine, losartan, mercaptopurine, rapamycin, or rifabutin (N=2 with n=8 replicates for each concentration) (one-way ANOVA with Dunnett's post-hoc test).

FIGS. 6A-6C. Acetaminophen, caffeine, losartan, rapamycin, and rifabutin reduce coiling due to α-synuclein-mediated dopaminergic neuron dysfunction in vivo. (a) Coiler scores of syn animals and GFP animals treated with vehicle or 100 μM of drug, normalized to vehicle treated control GFP animals (N=12 populations of n=10 animals per treatment group) (one-way ANOVA with Tukey's post-hoc test, ** p<0.01, *** p<0.001, **** p<0.0001). (b) Locomotory speed of syn animals treated with vehicle or 100 μM of drug, normalized to vehicle treated control GFP animals (N=12 populations of n=10 animals per treatment group, two-tailed t-test, * p<0.05). (c) No statistically significant changes in coiler scores of unc-10(e102) animals treated with vehicle or 100 μM of drug, normalized to vehicle treated unc-10(e102) animals (N=12 populations of n=10 animals per treatment group) (one-way ANOVA with Tukey's post-hoc test). Each data point represents an individual population.

FIGS. 7A-7E. Losartan, rapamycin, and rifabutin reduce α-synuclein oligomers in mammalian neurons. (a) Five positive hits were tested for their ability to reduce α-synuclein oligomer levels in rat primary cortical neurons measured with YFP-based protein-fragment complementation. No statistically significant changes were observed in (b) total α-synuclein levels measured by immunohistochemistry or (c) fluorescence of full-length YFP alone. Fluorescence intensity was analyzed using Imaris image analysis software by measuring the mean fluorescence intensity of α-synuclein positive neurons throughout a 3D field of view generated by z-stack image acquisition using the “surfaces” module. The value generated for each neuron was normalized to the mean fluorescence intensity of neurons treated with vehicle alone (N=3-4 replicates of n=10 fields of view for each treatment; each data point represents an individual replicate) (nested one-way ANOVA with Dunnett's post-hoc test, *p<0.05, *** p<0.001). (d) Top & Middle: Representative fluorescent microscopy images of neurons treated with vehicle, losartan, rapamycin, or rifabutin and co-expressing human α-synuclein tagged with the N- or C-terminal half of YFP. Bottom: Representative fluorescent microscopy images of neurons treated with vehicle, losartan, rapamycin, or rifabutin and expressing YFP alone. Scale bars are 10 μM. (e) Representative immunoblot to detect oligomers of split YFP-tagged α-synuclein (crosslinked) in rat primary cortical neuron lysates (actin used as a loading control).

FIGS. 8A-8F. Rifabutin reduces nigrostriatal dopaminergic neurodegeneration in an AAV-based α-synuclein rat model. (a) Animals treated with rifabutin demonstrated less dopaminergic (TH+) cell death in the SN compared with vehicle treated animals (n=8 animals per treatment group, two-tailed t-test, *p<0.05). (b) Representative images of TH staining of the SN injected with AAV-A53 T α-synuclein and the contralateral non-injected SN from rats treated with vehicle or rifabutin. Scale bars are 200 μm. (c) Striatal levels of dopamine (DA) and its metabolites, DOPAC and HVA, were higher for rifabutin treated animals compared with vehicle treated animals (n=8 animals per treatment group, two-tailed t-test, *p<0.05). (d) Analyses of immunofluorescent images demonstrated that animals treated with rifabutin exhibited less dopaminergic (TH+) cell death in the injected SN compared to vehicle treated animals. Levels of α-synuclein oligomers and total human α-synuclein were lower in the surviving TH+ cells in the SN of rifabutin treated animals versus vehicle treated animals (n=7-8 animals per treatment group, two-tailed t-test, *p<0.05). (e) Representative images of immunofluorescent staining with anti-TH antibodies (to detect dopaminergic neurons), Syn-O2 (to detect α-synuclein oligomers), or Syn211 (to detect total human α-synuclein) in SN injected with AAV-A53 T α-synuclein from rats treated with vehicle or rifabutin. Scale bars are 200 μm. (f) RT-QuIC spectra of CSF from 7 animals treated with vehicle and 8 animals treated with rifabutin. The dotted black line represents the threshold (mean background fluorescence+5 SD) defining the positive and negative cases. Four vehicle treated cases were positive and 3 vehicle treated cases were negative (gray spectra). All 8 rifabutin treated cases were negative.

FIGS. 9A-9B. Performance of the algorithm used to rank candidate drugs by similarity to the known entities that reduce α-synuclein oligomers. (a) Receiver-operating characteristic (ROC) curve. The semantic framework was assessed using leave-one-out validation: one known entity was placed among the other candidate drugs and ranked, repeating for each individual drug in the known set. Individual points were connected using a stepwise curve. The area under curve (AUC) value is 0.98. A dashed line, with an AUC score of 0.5, represents a null hypothesis curve where the rankings of known entities made by IBM Watson for Drug Discovery Predictive Analytics would be completely random. (b) Precision-recall curve. The curve has an average precision of 0.50 and is always above the dashed line, a precision of 0.02 (the percent of positives), which represents a model with no skill.

FIG. 10. Acetaminophen, caffeine, losartan, mercaptopurine, rapamycin, and rifabutin do not alter the activity of full-length Gaussia luciferase alone. Raw luminescence data (measured in relative luciferase units) corresponding with normalized data in FIG. 5c are shown here (N=2 with n=8 replicates for each concentration).

FIGS. 11A-11D. Effects of acetaminophen, caffeine, losartan, mercaptopurine, rapamycin, and rifabutin on α-amyloid and tau oligomers in vitro. Testing of compounds found to reduce α-synuclein oligomers for their effects on (a) β-amyloid oligomer levels in vitro using a Gaussia luciferase protein-fragment complementation cell assay (N=3 with n=8 replicates for each treatment) and (b) viability in these cells (N=3 with n=4 replicates for each treatment). Testing of compounds found to reduce α-synuclein oligomers for their effects on (c) tau oligomer levels in vitro using a Gaussia luciferase protein-fragment complementation cell assay (N=3 with n=8 replicates for each treatment) and (d) viability in these cells (N=3 with n=4 replicates for each treatment) (two-tailed t-test, *p<0.05, ** p<0.01, **** p<0.0001). Each data point represents an individual replicate.

FIGS. 12A-12B. Abacavir, Acebutolol, Benzydamine, Hydralazine, Clioquinol, Lamivudine and Modafinil reduce α-syn oligomers. (A-B) Testing of the top 19 highly ranked drug compounds (1 μM or 10 μM) for their ability to reduce α-syn levels in vitro using a Gaussia luciferase protein fragment complementation assay in H4 neuroglioma cells. Rapamycin (2 μM) and bafilomycin A1 (50 nM) were used as positive and negative controls respectively. n=4 for each treatment group (one-tailed t-test, p<0.05*).

FIGS. 13A-13G. Abacavir, Lamivudine and Acebutolol are non-toxic in cells. H4 neuroblastoma following treatment of drugs (1-20 μM), measured using Presto Blue-mediated fluorescence, and normalized to vehicle alone. (a, b & e) No statistically significant changes in viability were observed following Abacavir, Lamivudine and Acebutolol treatment at <10 μM in H4 cells. Significant loss in cell viability was observed in (c) Benzydamine, 1.25 μM (f) Modafinil, <5 μM and (d, g) Clioquinol and Hydralazine, <10 μM (1way ANOVA with Dunnett's multiple comparisons test, p<0.05*, p<0.01 **, p<0.001 ***, p<0.0001 ****).

FIGS. 14A-14C. Gaussia luciferase activity following drug treatment in H4 cells. No statistically significant changes in the activity of full-length Gaussia luciferase alone were found with Acebutolol, Lamivudine or Abacavir (10 μM; n=2 with n=8 replicates for each concentration, one-way ANOVA with Dunnett's multiple comparisons test).

FIGS. 15A-15E. Abacavir is able to significantly reduce α-syn oligomers and increase HSP70 in primary rat cortical neurons. (a) Representative fluorescence microscopy images of positive hits were tested for their ability to reduce (b) α-syn oligomers (c) total α-syn levels (d) fluorescence of full-length YFP alone and their effect on (e) fluorescence intensity of HSP70. Abacavir treatment (10 μM) significantly reduced α-syn oligomers (a) compared to vehicle control (1way ANOVA p<0.0001 ***, one-tailed t-test, p<0.081 ***) and significantly increase HSP70 compared to vehicle dimethyl sulfoxide (DMSO) control (1way ANOVA p<0.0001 ***, one-tailed t-test, p<0.081 ***). Fluorescence intensity was normalized to vehicle alone for each treatment (n=3 replicates of n=10 fields of view per treatment).

FIGS. 16A-16B. Abacavir reduces α-synuclein-induced coiling due to dopaminergic neuron dysfunction in C. elegans. (a) Coiler scores of syn animals and GFP animals treated with vehicle or 100 μM of abacavir, normalized to vehicle treated control GFP animals (n=12 populations of n=10 animals per treatment group) (one-way ANOVA with Tukey's post-hoc test, *p<0.05). (b) Locomotory speed of syn animals treated with vehicle or 100 μM of drug, normalized to vehicle treated control GFP animals (n=12 populations of n=10 animals per treatment group, one-way ANOVA with Tukey's post-hoc test.

FIGS. 17A-17D. Rifabutin penetrates the blood-brain barrier and reduces nigral dopaminergic neuron loss in an AAV-based α-synuclein rat model. (a) Dopaminergic neuron loss in the SN was induced by stereotactic injection of human A53 T α-synuclein-expressing AAV (syn). AAV lacking the A53 T α-synuclein open reading frame was used as an empty vector control (EV). Animals injected with AAV-A53 T α-synuclein and treated with vehicle exhibited a substantial loss of dopaminergic (TH+) cells. Rifabutin rescued α-synuclein-mediated dopaminergic neurodegeneration in the SN, restoring TH+ cell numbers to those comparable to animals injected with AAV-EV and treated with vehicle or rifabutin (n=8-11 animals per treatment group, one-way ANOVA with Tukey's post-hoc test, *** p<0.001). (b) Representative images of TH staining of the SN injected with AAV-A53 T α-synuclein or AAV-EV from rats treated with vehicle or rifabutin. Scale bars are 200 μm. Steady-state rifabutin concentrations in (c) plasma and (d) saline-perfused brain tissue from a subset of animals (n=4 per treatment group). Mean brain-to-plasma drug concentration ratio (±SD) for rifabutin treated animals was 0.15±0.04.

FIG. 18. Abacavir increases HSP70 mRNA levels in rat primary cortical neurons. Rat primary cortical neurons were treated with vehicle control (DMSO) or abacavir (10 μM). Real-time quantitative RT-PCR was performed with 3 distinct primer sets for HSP70. β-Actin was used as the reference gene and the expression of HSP70 mRNA was analyzed by 2^{-ΔΔCT} method. The control group treated with DMSO was set as calibrator sample representing the 1×expression. Data are presented as mean±SEM. (n=3 independent experiments, unpaired two-tailed Student's t-test, ** p<0.01)

### DETAILED DESCRIPTION OF THE DISCLOSURE

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, Green, M. and Sambrook, J. (2012) Molecular Cloning: A Laboratory Manual. 4th Edition, Vol. II, Cold Spring Harbor Laboratory Press, New York).

### Definitions

As used herein, the term “subject” refers to a mammalian subject, preferably a human.

As used herein, the term “rifabutin”, which is an antimycotic antibiotic typically used to treat tuberculosis, refers to a compound that has the following chemical structure:

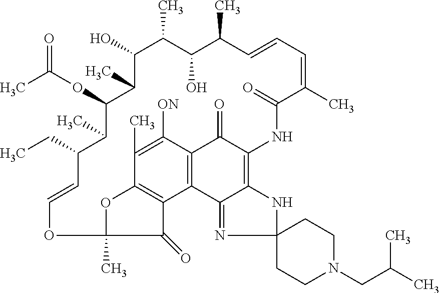

as well as any pharmaceutically acceptable salts thereof, and includes various formulations of rifabutin including generic formulations and brand name formulations such as Mycobutin.

As used herein, the term “losartan”, which is an angiotensin II receptor blocker (ARB) typically used to treat high blood pressure, refers to a compound that has the following chemical structure:

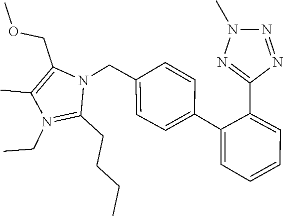

as well as any pharmaceutically acceptable salts thereof, for example losartan potassium and includes various formulations of losartan including generic formulations and brand name formulations such as Cozaar.

As used herein, the term “abacavir” refers to a nucleoside reverse transcriptase inhibitor (NRTI) typically used to treat human immunodeficiency virus (HIV) infection, and has the following chemical structure:

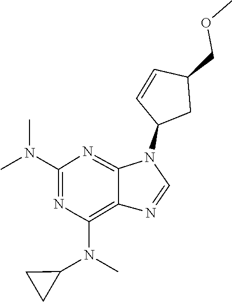

as well as any pharmaceutically acceptable salts thereof and includes various formulations of abacavir including generic formulations and brand name formulations such as Ziagen.

As used herein, the term “lamiduvine”, which is a nucleoside reverse transcriptase inhibitor (NRTI) typically used to treat human immunodeficiency virus (HIV) and hepatitis B infection, refers to a compound that has the following chemical structure:

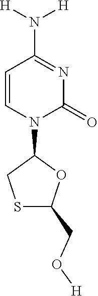 [SMILES: Nc1ccn([C@@H]2CS[C@H](CO)O2)c(=O)n1]

as well as any pharmaceutically acceptable salts thereof and refers to various formulations of abacavir including generic formulations and brand name formulations such as Epivir or 3TC.

As used herein, the terms “treat” or “treating”, as used herein, unless otherwise indicated, mean reversing, alleviating, or inhibiting the progression of, the disorder or condition to which such term applies, or one or more symptoms of such disorder or condition. The term “treatment”, as used herein, unless otherwise indicated, refers to the act of treating, as defined immediately above.

As used herein, “NRTI” or “nucleoside reverse transcriptase inhibitor” or “nucleoside analog reverse transcriptase inhibitor” refers to a class of drugs, which for example have a chain terminating effect, including for example, lamivudine, emtricitabine, tenofovir disoproxil funiarate, tenofovir alafenamide, abacavir, zidovudine, didanosine, and stavudine.

As used herein, a patient who is “high risk” refers to a patient that has an increased risk of developing Parkinson's Disease or another α-synucleopathy as compared to a control population, has known genetic risk factor(s) or is an individual with prodromal PD (including those with idiopathic/isolated REM sleep behaviour disorder) for which there are research criteria, an example of which can be found in Heinzel et al, 2019, which is hereby incorporated by reference.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

An early motor phenotype of transgenic C. elegans expressing α-synuclein in dopaminergic neurons was identified. It is demonstrated herein that this motor abnormality is due to α-synuclein-mediated dopaminergic neuron dysfunction and occurs prior to neuronal loss. This in vivo model was applied downstream of a combined artificial intelligence (AI)-driven in silico and in vitro screening platform to identify compounds that reduce the C. elegans motor impairment. Described herein is a validated assay for the discovery of potential disease-modifying drugs for early intervention in PD and other α-synucleinopathies.

Using this assay, the inventors have identified several known drugs that could be repurposed for the treatment of PD.

One aspect of the disclosure is a method of treating a subject with an α-synucleinopathy neurodegenerative disorder, the method comprising administering one or more therapeutic(s) to the subject, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof. In some embodiments, the one or more therapeutic(s) is selected from the group comprising rifabutin, lamivudine, losartan, and abacavir. In some embodiments, the one or more therapeutic(s) is selected from the group comprising rifabutin, losartan, and abacavir. In another embodiment, the therapeutic is or comprises abacavir and lamivudine.

In a further embodiment, the subject is a mammal. In another embodiment, the subject is a human. For neurodegenerative diseases, such as PD, it is expected that disease-modifying therapies would be more effective if given as early as possible in the disease course. In one embodiment, the one or more therapeutic(s) is administered to individuals at high risk of developing PD prior to a clinical diagnosis including individuals, for example with known genetic risk factor(s) or individuals with prodromal PD (including those with idiopathic/isolated REM sleep behaviour disorder) for which there are research criteria, an example of which can be found in Heinzel et al, 2019, which is hereby incorporated by reference, or individuals who have been predicted to be high risk by an algorithm, for example as described in Noyce, Alastair J et al. “PREDICT-PD: An online approach to prospectively identify risk indicators of Parkinson's disease.” Movement disorders: official journal of the Movement Disorder Society vol. 32,2 (2017): 219-226. doi: 10.1002/mds.26898, which is incorporated herein by reference.

In another embodiment, the subject has wild type alpha synuclein. In a further embodiment, the subject carries a genetic mutation in alpha synuclein. In a further embodiment, the mutation in alpha synuclein is an A30P mutation, A53 T mutation, A30G mutation, A53E mutation, E83Q mutation, G51D mutation, H50Q mutation or E46K mutation.

In some embodiments, the synucleinopathy neurodegenerative disorder is selected from a group comprising a group comprising Parkinson's Disease, dementia with Lewy bodies, multiple system atrophy, and Alzheimer's disease, prodromal Parkinson's Disease, prodromal dementia with Lewy bodies, prodromal multiple system atrophy, and prodromal Alzheimer's disease (including those with idiopathic/isolated REM sleep behaviour disorder). In an embodiment, the α-synucleinopathy neurodegenerative disorder is selected from a group comprising Parkinson's Disease, dementia with Lewy bodies, multiple system atrophy, and Alzheimer's disease. In a further embodiment, the mutation in alpha synuclein is an A35 T mutation, A30P mutation, A30G mutation, A53E mutation, E83Q mutation, G51D mutation, H50Q mutation or E46K mutation. Any subset of disorders and/or mutations are contemplated. In another embodiment, the α-synucleinopathy neurodegenerative disorder is Parkinson's Disease.

In an embodiment, the mutation in alpha synulein is an A30P mutation. In another embodiment, the mutation in alpha synuclein is an A35 T mutation.

In some embodiments, the one or more therapeutic(s) administered are rifabutin, lamivudine, losartan, and/or abacavir. In some embodiments, the one or more therapeutic(s) are or comprise rifabutin, losartan, and abacavir. In some embodiments, the therapeutic is or comprises rifabutin, lamivudine, losartan or abacavir. In some embodiments, the therapeutic is or comprises rifabutin, losartan or abacavir. In further embodiments, the therapeutic is or comprises rifabutin. In another embodiment, the therapeutic is or comprises losartan. In another embodiment, the therapeutic is or comprises abacavir. In another embodiment, the therapeutic is or comprises lamivudine. In another embodiment, the therapeutic is or comprises abacavir and lamivudine. In other embodiments, the method further comprises administering rapamycin. In some embodiments, the rapamycin is administered prior to, simultaneously, or after, the one or more therapeutic(s) rifabutin, losartan, lamivudine, and/or abacavir. Any subset of therapeutics are contemplated.

In some embodiments, the subject is administered the one or more therapeutic(s) immediately following a clinical diagnosis of Parkinson's Disease, dementia with Lewy bodies, multiple system atrophy, Alzheimer's disease. In some embodiments, rapamycin is administered prior to, simultaneously, or after, the one or more therapeutic(s) rifabutin, lamivudine, losartan, and/or abacavir. Any subset of disorders and/or therapeutics are contemplated.

Another aspect of the disclosure is a method of treating a subject with a high risk of developing an α-synucleinopathy neurodegenerative disorder, the method comprising administering one or more therapeutic(s) to the subject, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof. In some embodiments, the one or more therapeutic(s) is selected from the group comprising rifabutin, lamivudine, losartan, and abacavir. In some embodiments, the one or more therapeutic(s) is selected from the group comprising rifabutin, losartan, and abacavir. In another embodiment, the therapeutic is or comprises abacavir and lamivudine. In some embodiments, the subject has known risk factor(s) for developing an α-synucleinopathy neurodegenerative disorder, including genetic risk factor(s) identified by GWAS (such as those described in Nalls et al., 2019 and Chia et al., 2021 which are incorporated herein by reference), environmental risk factor(s) identified by meta-analyses (such as those described in Noyce et al., 2012 and Bellou et al., 2016 which are incorporated herein by reference), or the subject has increased risk for developing an α-synucleinopathy neurodegenerative disorder based prediction algorithms (such as Noyce et al., 2017 and, Schlossmacher et al., 2017 which are incorporated herein by reference). Any subset of therapeutics are contemplated.

In some embodiments, the α-synucleinopathy neurodegenerative disorder is selected from a group comprising Parkinson's Disease, dementia with Lewy bodies, multiple system atrophy, and Alzheimer's disease. In some embodiments, the α-synucleinopathy is Parkinson's Disease. In further embodiments, the subject has prodromal Parkinson's Disease, prodromal dementia with Lewy bodies, prodromal multiple system atrophy, and prodromal Alzheimer's disease (including those with idiopathic/isolated REM sleep behaviour disorder). Criteria for prodromal PD is well known in the art such as Berg et al., 2015 which is incorporated herein by reference, criteria for diagnosis of prodromal dementia with Lewy bodies is well known in the art such as McKeith et al., 2020 which is incorporated herein by reference, and criteria for prodromal Alzheimer's Disease is well known in the art such as Jack et al., 2018 which is incorporated herein by reference. Any subset of disorders are contemplated.

In some embodiments, the subject is administered the one or more therapeutic(s) prior to clinical diagnosis according to accepted diagnostic criteria of an α-synucleinopathy neurodegenerative disorder. Accepted diagnostic criteria for α-synucleinopathy neurodegenerative disorder are well known in the art, for example as in Postuma et al., 2015, McKeith et al., 2017, Gilman et al., 2008, and McKhann et al., 2011, each of which is incorporated herein by reference.

In a further embodiment, the subject is a mammal. In another embodiment, the subject is a human.

In another embodiment, the subject has wild type alpha synuclein. In a further embodiment, the subject carries a genetic mutation in alpha synuclein. In a further embodiment, the mutation in alpha synuclein is an A30P mutation.

In an embodiment, the α-synucleinopathy neurodegenerative disorder is selected from a group comprising Parkinson's Disease, dementia with Lewy bodies, multiple system atrophy, and Alzheimer's disease. In a further embodiment, the mutation in alpha synuclein is an A35 T mutation. In another embodiment, the α-synucleinopathy neurodegenerative disorder is Parkinson's Disease.

In some embodiments, the one or more therapeutic(s) administered are or comprise rifabutin, losartan, lamivudine, and/or abacavir. In some embodiments, the one or more therapeutic(s) are or comprise rifabutin, losartan, lamivudine, and abacavir. In some embodiments, the one or more therapeutic(s) are or comprise rifabutin, losartan, and abacavir. In some embodiments, the one or more therapeutic(s) is or comprises rifabutin, losartan, lamivudine, or abacavir. In some embodiments, the one or more therapeutic(s) is or comprises rifabutin, losartan, or abacavir. In further embodiments, the therapeutic is rifabutin. In another embodiment, the therapeutic is losartan. In another embodiment, the therapeutic is abacavir. In another embodiment, the therapeutic is lamivudine. In another embodiment, the therapeutic is or comprises abacavir and lamivudine. In other embodiments, the method further comprises administering rapamycin. In some embodiments, the rapamycinis administered prior to, simultaneously, or after, the one or more therapeutic(s) rifabutin, losartan, lamivudine, and/or abacavir. Any subset of therapeutics are contemplated.

In some embodiments, the subject is administered the one or more therapeutics immediately following a clinical diagnosis of Parkinson's Disease, dementia with Lewy bodies, multiple system atrophy, Alzheimer's disease. In some embodiments, rapamycin is administered prior to, simultaneously, or after, the one or more therapeutic(s) rifabutin, losartan, and/or abacavir. In another embodiment, the method comprises administering losartan and rifabutin. In another embodiment, the method comprises administering abacavir and lamivudine. Any subset of disorders and/or therapeutics are contemplated.

In another embodiment, losartan, rifabutin, lamivudine, and/or abacavir are administered orally, intraperitoneally, via injection, intranasally, intrathecally, and/or intraventricularly. In some embodiments rapamycin is also administered orally, intraperitoneally, via injection, intranasally, intrathecally, and/or intraventricularly. In some embodiments, the losartan, rifabutin, lamivudine, and/or abacavir is administered orally.

Another aspect of the disclosure includes a method of inhibiting α-synuclein aggregation, the method comprising contacting a cell with one or more therapeutic(s), wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof. In some embodiments, the one or more therapeutic(s) is selected from the group comprising rifabutin, lamivudine, losartan, and abacavir. In some embodiments, the one or more therapeutic(s) is selected from the group comprising rifabutin, losartan, and abacavir. In another embodiment, the therapeutic is or comprises abacavir and lamivudine. In some embodiments, the one or more therapeutic(s) selected from a group comprising rifabutin, lamivudine, losartan, and abacavir. In some embodiments, the one or more therapeutic(s) selected from a group comprising rifabutin, losartan, and abacavir. In some embodiments, the one or more therapeutic(s) comprises abacavir and lamuvidine. In some embodiments, the cell is a mammalian cell. In some embodiments, the cell is a human cell. In some embodiments, the cell is a neuron. In some embodiments, the cell is in a subject afflicted with or at risk of developing an α-synucleinopathy neurodegenerative disorder. In some embodiments, the α-synucleinopathy neurodegenerative disorder is selected from Parkinson's Disease, dementia with Lewy bodies, multiple system atrophy, or Alzheimer's disease. In some embodiments, the subject has prodromal Parkinson's Disease, prodromal dementia with Lewy bodies, prodromal multiple system atrophy, and prodromal Alzheimer's disease (including those with idiopathic/isolated REM sleep behaviour disorder). For example, contacting includes administering to a subject, when the cell is in a subject. As another example α-synuclein aggregation can include intracellular and/or extracellular aggregation. Any subset of disorders and/or therapeutics are contemplated.

In some embodiments, the subject is administered the one or more therapeutic(s) prior to clinical diagnosis of an α-synucleinopathy neurodegenerative disorder, for example a subject identified with prodromal Parkinson's Disease. In some embodiments, the one or more therapeutic(s) are rifabutin, lamivudine, losartan, and abacavir. In some embodiments, the one or more therapeutic(s) are rifabutin, losartan, and abacavir. In some embodiments, the therapeutic is rifabutin. In some embodiments, the therapeutic is losartan. In some embodiments, the therapeutic is abacavir. In some embodiments, the therapeutic is lamivudine. In some embodiments, the method further comprises administering rapamycin. Also provided are uses of any of the methods provided herein for example, use of one or more therapeutic(s) for treating a subject with an α-synucleinopathy neurodegenerative disorder, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof. In some embodiments, the one or more therapeutic(s) is selected from the group comprising rifabutin, lamivudine, losartan, and abacavir. In some embodiments, the one or more therapeutic(s) is selected from the group comprising rifabutin, losartan, and abacavir. In another embodiment, the therapeutic is or comprises abacavir and lamivudine. In some embodiments, the one or more therapeutic(s) is selected from rifabutin, losartan and abacavir. Another example includes use of one or more therapeutic(s) for treating a subject with a high risk of developing an α-synucleinopathy neurodegenerative disorder, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof. In some embodiments, the one or more therapeutic(s) is selected from the group comprising rifabutin, lamivudine, losartan, and abacavir. In some embodiments, the one or more therapeutic(s) is selected from the group comprising rifabutin, losartan, and abacavir. In another embodiment, the therapeutic is or comprises abacavir and lamivudine. Another example includes use one or more therapeutic(s) in the manufacture of a medicament for the treatment of an α-synucleinopathy neurodegenerative disorder, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof. In some embodiments, the one or more therapeutic(s) is selected from the group comprising rifabutin, lamivudine, losartan, and abacavir. In some embodiments, the one or more therapeutic(s) is selected from the group comprising rifabutin, losartan, and abacavir. In another embodiment, the therapeutic is or comprises abacavir and lamivudine. A further example includes use of one or more therapeutic(s) in the manufacture of a medicament for the treatment of an α-synucleinopathy neurodegenerative disorder, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof. In some embodiments, the one or more therapeutic(s) is selected from the group comprising rifabutin, lamivudine, losartan, and abacavir. In some embodiments, the one or more therapeutic(s) is selected from the group comprising rifabutin, losartan, and abacavir. In another embodiment, the therapeutic is or comprises abacavir and lamivudine. Any subset of therapeutics are contemplated.

Another example includes use of one or more therapeutic(s) for inhibiting α-synuclein aggregation, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof. In some embodiments, the one or more therapeutic(s) is selected from the group comprising rifabutin, lamivudine, losartan, and abacavir. In some embodiments, the one or more therapeutic(s) is selected from the group comprising rifabutin, losartan, and abacavir. In another embodiment, the therapeutic is or comprises abacavir and lamivudine. Another example includes one or more therapeutic(s) for inhibiting α-synuclein aggregation, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof. In some embodiments, the one or more therapeutic(s) is selected from the group comprising rifabutin, lamivudine, losartan, and abacavir. In some embodiments, the one or more therapeutic(s) is selected from the group comprising rifabutin, losartan, and abacavir. In another embodiment, the therapeutic is or comprises abacavir and lamivudine. Another example includes one or more therapeutic(s) for treating an α-synucleinopathy neurodegenerative disorder, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof. In some embodiments, the one or more therapeutic(s) is selected from the group comprising rifabutin, lamivudine, losartan, and abacavir. In some embodiments, the one or more therapeutic(s) is selected from the group comprising rifabutin, losartan, and abacavir. In another embodiment, the therapeutic is or comprises abacavir and lamivudine. Any subset of therapeutics are contemplated.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

To identify early motor impairment of transgenic animals expressing α-synuclein in dopaminergic neurons, a custom-built tracking microscope that captures locomotion of single C. elegans with high spatial and temporal resolution was used. Next, a method for semi-automated and blinded quantification of motor impairment for a population of simultaneously recorded animals with multi-worm tracking and custom image processing was devised. Genetic and pharmacological methods were then used to define the features of early motor dysfunction of α-synuclein-expressing C. elegans. Finally, we applied the C. elegans model to a drug repurposing screen by combining it with an artificial intelligence platform and cell culture systems to identify small molecules that inhibit α-synuclein oligomers.

A previously undescribed motor phenotype in C. elegans expressing α-synuclein that correlates with mutant or wild-type α-synuclein protein levels and results from dopaminergic neuron dysfunction but precedes neuronal loss was identified. Together with artificial intelligence-driven in silico and in vitro screening, this in vivo model identified five compounds that reduced motor dysfunction induced by α-synuclein. Three of these compounds also decreased α-synuclein oligomers in mammalian neurons.

Methods are described in Example 6.

### Example 2

Coiling is an Early Motor Impairment of C. elegans Expressing Mutant α-Synuclein

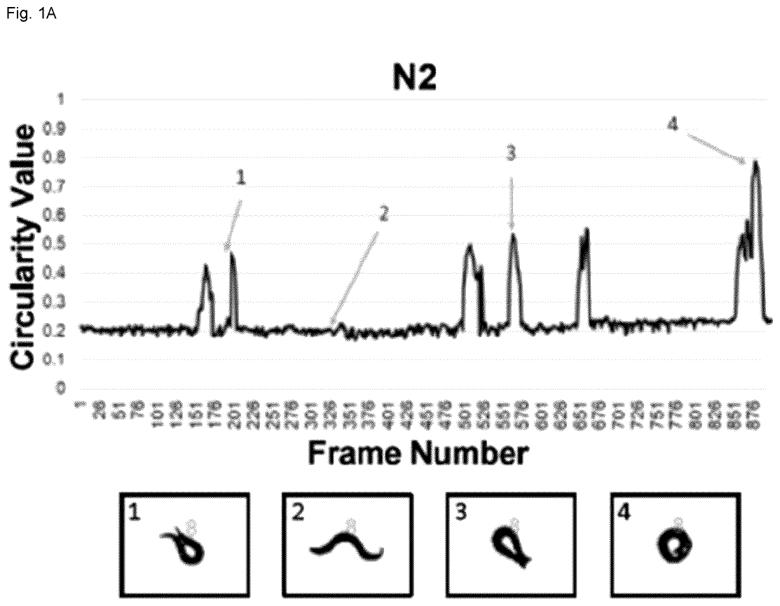

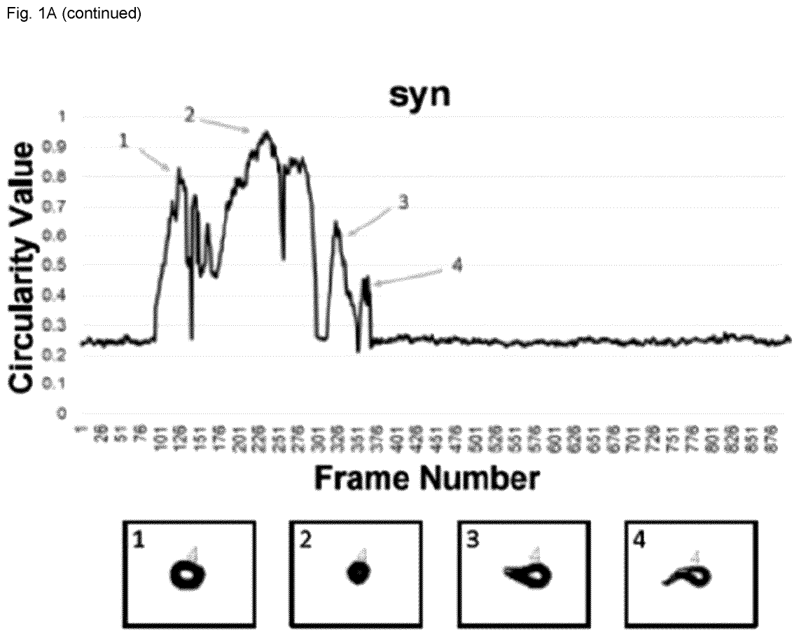

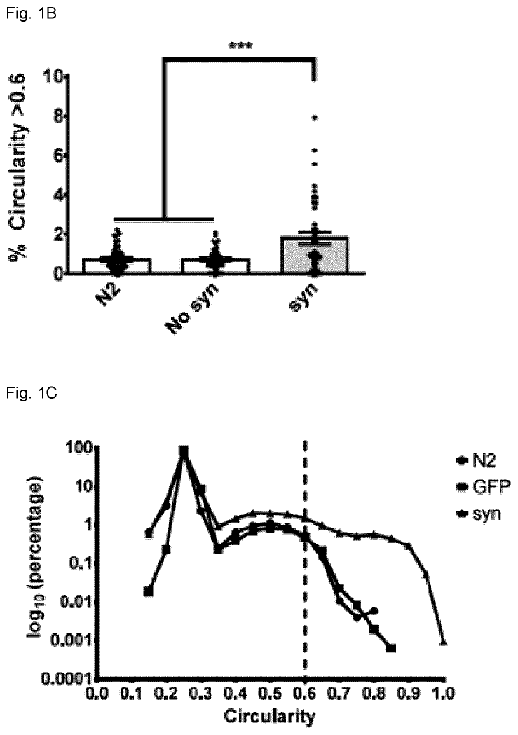

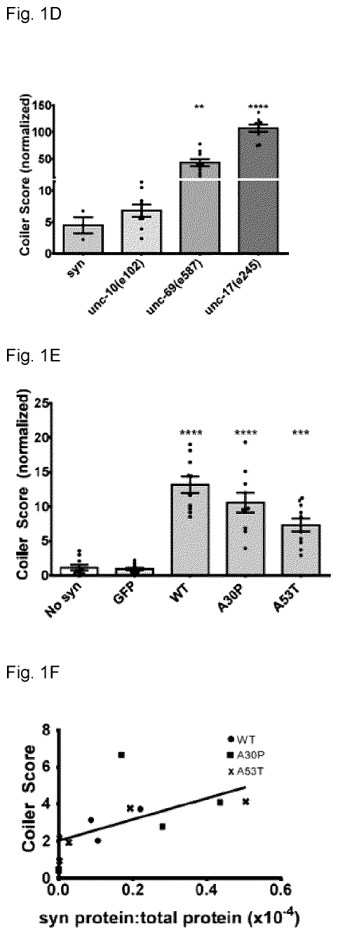

Expression of α-synuclein in dopaminergic neurons of C. elegans has not been previously reported to cause visible defects in spontaneous crawling. A subtle uncoordinated (unc) “coiler” phenotype was observed among transgenic C. elegans expressing a mutant form of human α-synuclein, with alanine at amino acid position 30 mutated to proline (A30P), under control of the dopamine neuron specific promoter, dat-1p. The A30P mutation is a known cause of inherited monogenic PD.² Coiling is an abnormal motor behaviour in which the animal spends increased time with its body in a round or circular position. To measure this motor impairment, a custom-built tracking microscope that captures and follows the motions of single C. elegans with high spatial and temporal resolution to record their locomotion was used. Coiling was quantified by calculating a numerical value for circularity, which corresponds with C. elegans adopting a circular shape. The circularity value is based on the formula 4π(A/P²), where A is area and P is perimeter of the worm's body. A value of 1 indicates a perfect circle and a value approaching 0 indicates an increasingly elongated shape. Recordings of individual transgenic C. elegans expressing A30P α-synuclein or non-transgenic N2 C. elegans revealed that the normal sinusoidal shape of the animals corresponded to circularity values of ˜0.2. The body shape during an omega turn, which is a normal motor behaviour used by the animal to change directions, corresponded to circularity values of ˜0.5, whereas coiling animals had circularity values ranging from ˜0.6 to almost 1 (FIG. 1a). A30P α-synuclein C. elegans were found to have circularity values in the coiling range for a greater proportion of video frames than non-transgenic N2 animals or C. elegans with the same genetic background (tzls3) but expressing no α-synuclein (FIG. 1b). Further, N2 and tzls3 animals showed the same degree of circularity, suggesting that tzls3 did not affecting coiling.

Using high-resolution, multi-worm tracking with custom image processing, methods were developed for semi-automated and blinded scoring of circularity for a population of simultaneously recorded animals (n=10 per recording). A coiler score was determined for the population tested by first calculating the circularity value for each animal in each frame of video and then calculating the proportion of circularity values >0.6. A circularity value threshold of 0.6 was chosen to isolate the coiler phenotype because it was found that, for N2 animals or control animals expressing green fluorescent protein (GFP) instead of α-synuclein in dopaminergic neurons, the likelihood of circularity values >0.6 was very low compared to A30P α-synuclein animals (FIG. 1c). The coiler phenotype is an abnormal motor behaviour previously reported for several unc C. elegans strains with mutations affecting neuronal function. To test the validity of the methods described herein for scoring the coiler phenotype, three C. elegans strains with differing degrees of coiling due to presynaptic dysfunction were examined: 1) unc-10 (e102), a mild coiler with a mutation in an ortholog of human RIMS1 (regulating synaptic membrane exocytosis 1) which is involved in presynaptic vesicular priming;¹⁴ 2) unc-69(e587), a moderate coiler with a mutation in an ortholog of human SCOCO (short coiled-coil protein) which functions in axonal outgrowth and presynaptic organization;¹⁵ and, 3) unc-17(e245), a severe coiler with a mutation in an ortholog of human SLC18A3 (solute carrier family 18 member A3) which exhibits acetylcholine transmembrane transporter activity.¹⁶ The mean (±SEM) coiler scores for unc-10(e102), unc-69(e587), and unc-17(e245), normalized to control GFP animals, were found to be 6.7±1.0, 43.2±6.6, and 107.8±7.0, respectively (FIG. 1d). The normalized mean (±SEM) coiler score for animals expressing A30P α-synuclein was 4.5±1.3, similar to that of unc-10(e102). These results demonstrate that the methods described herein can quantify the coiler phenotype of a C. elegans population and appropriately differentiate between different degrees of coiling. These animals were examined as young adults (4 days post-hatching) but morphological evidence of dopaminergic neurodegeneration in C. elegans expressing α-synuclein is reported to occur in older adults (6 to 10 days post-hatching).¹⁰ Therefore, these findings suggest that mild coiling is an early motor phenotype in C. elegans expressing A30P α-synuclein exclusively in dopaminergic neurons which can be measured using the methods described herein for semi-automated and blinded scoring of a population of animals.

Methods are described in Example 6.

### Example 3

Early Motor Impairment Correlates with Mutant or Wild-Type α-Synuclein Protein Levels

To determine whether the coiler phenotype discovered here for C. elegans expressing mutant A30P α-synuclein was a motor abnormality of other transgenic α-synuclein C. elegans, the locomotion of C. elegans expressing mutant A53 T or wild-type α-synuclein only in dopaminergic neurons. Transgenic A30P, A53 T, and wild-type α-synuclein C. elegans demonstrated comparable coiler scores which were each higher than the coiler score for control C. elegans expressing GFP instead of α-synuclein in dopaminergic neurons or animals without expression of ectopic protein in those neurons (FIG. 1e).

A decline in locomotion occurs as C. elegans age and hence motor impairment may correlate with the animal's lifespan. For example, C. elegans with mutations that prolong lifespan have slower motor decline, whereas the decline is accelerated in animals with mutations that reduce longevity.¹⁷ Since ectopic expression of α-synuclein in C. elegans has been reported to have variable effects on lifespan,¹⁸⁻²⁰ we measured lifespans of the transgenic A30P, A53 T, and wild-type α-synuclein C. elegans were measured. Their mean (±SD) lifespans were comparable at 16.3±0.5, 16.1±1.2, and 17.9±1.2 days, respectively, and were not statistically significantly shorter than that of control GFP animals (16.7±0.9 days). Thus, a shorter lifespan could not account for the motor dysfunction observed in these transgenic α-synuclein C. elegans.

Because the copy number of transgenes can vary between each C. elegans line, whether the differences in coiling between the transgenic α-synuclein animals were related to α-synuclein protein expression levels was tested. The coiling behaviour and α-synuclein protein levels of 12 independent transgenic C. elegans lines were measured: 9 expressing α-synuclein in dopaminergic neurons, and 3 co-expressing α-synuclein and GFP in dopaminergic neurons. A positive correlation between coiling severity and α-synuclein protein expression was observed (FIG. 1f). Together these findings indicate that coiling is not simply an artefact of ectopic protein expression in dopaminergic neurons since this motor phenotype occurred with α-synuclein but not GFP. Analogous to PD in humans, a more severe phenotype is associated with higher expression of α-synuclein.²¹ It was found that expression in dopaminergic neurons of either mutant α-synuclein (associated with familial PD) or wild-type α-synuclein (associated with sporadic PD) caused coiling. Therefore, for the remainder of the present study, the transgenic A30P α-synuclein C. elegans were used.

Methods are described in Example 6.

### Example 4

Early Motor Impairment Results from Dopaminergic Neuron Dysfunction Due to α-Synuclein and Precedes Neuronal Loss

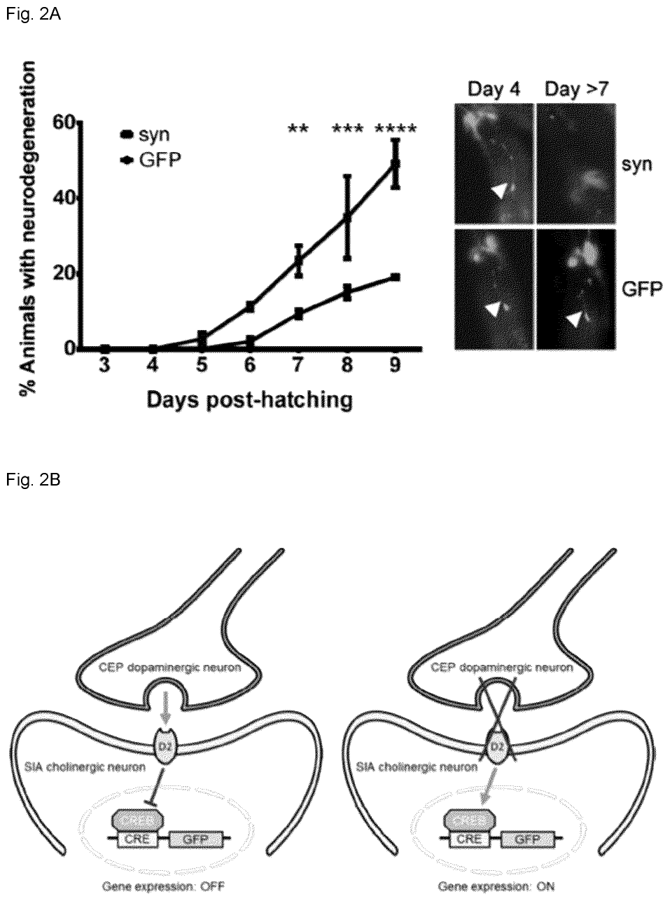

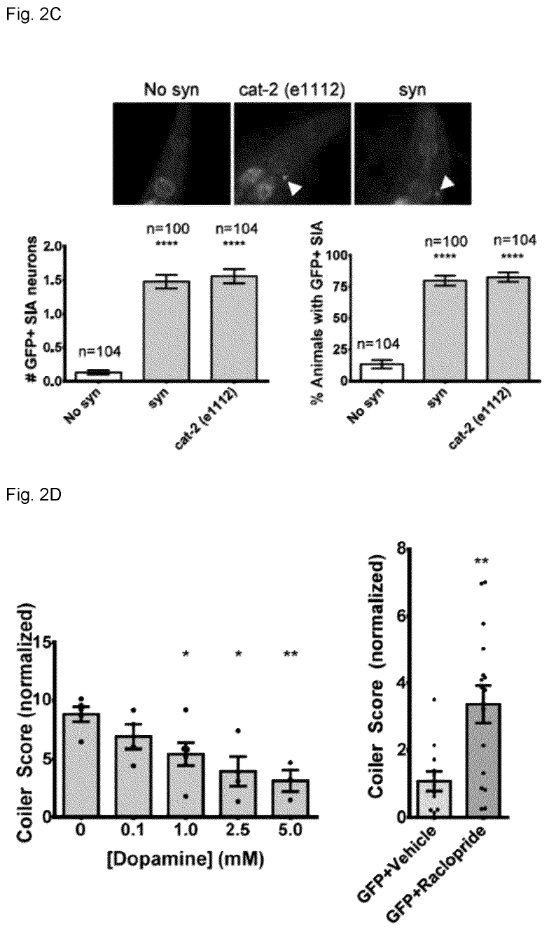

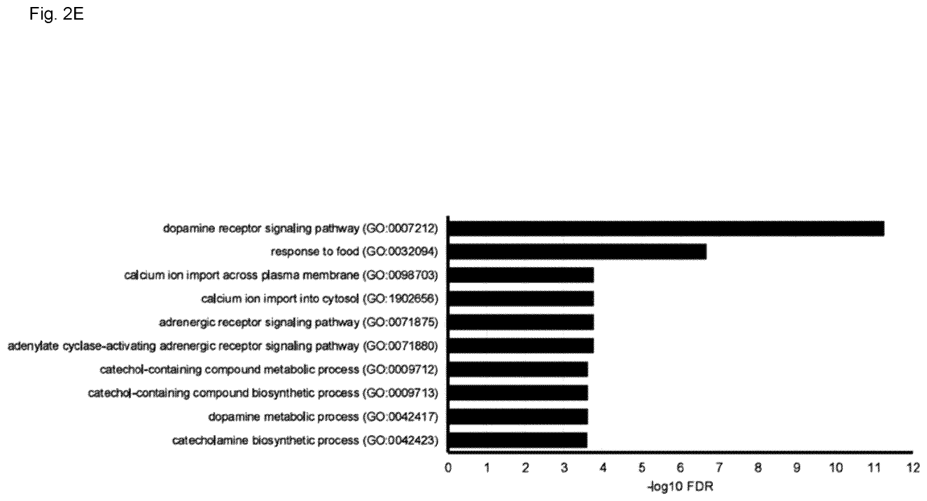

When administered to mammals, 1-methyl-4-phenyl-1,2,3,6-tetrahydropyridine (MPTP) crosses the blood-brain barrier and is converted in the brain to its toxic metabolite 1-methyl-4-phenylpyridinium (MPP+), which causes selective degeneration of dopaminergic neurons in the substantia nigra pars compacta by inhibiting the respiratory chain enzyme complex I in mitochondria. Since the intent was to develop a model that recapitulates early stages of neurodegeneration, whether coiling of transgenic A30P α-synuclein C. elegans occurred prior to dopaminergic neuron loss was investigated, which is typical of late-stage disease. A C. elegans hermaphrodite has 8 dopaminergic neurons, each with a stereotyped location: 2 pairs of cephalic (CEP) neurons and 1 pair of anterior deirid (ADE) neurons in the head region, and 1 pair of posterior deirid (PDE) neurons in the posterior body region. Because C. elegans are transparent, fluorescent markers are easily visualized in vivo and thus GFP (dat-1p::gfp) were used to track dopaminergic neuron loss over time. It was found that degeneration of ADE neurons are the easiest of the head dopaminergic neurons to visualize and therefore the most reliable to score. No loss of ADE neurons was observed in animals co-expressing A30P α-synuclein and GFP at the age when coiling was measured (4 days post-hatching). For older adults (from day 7 onwards), a statistically significant proportion of A30P α-synuclein animals exhibited ADE neurodegeneration, compared with animals expressing GFP alone (FIG. 2a). Therefore, coiling of A30P α-synuclein animals precedes dopaminergic neuron loss.

To determine whether dopaminergic neuron dysfunction, in the absence of neuronal loss, could account for the motor impairment of A30P α-synuclein animals, a fluorescent reporter system as an indicator of dopaminergic neuron signalling in C. elegans was used.^{25,26} In this system, C. elegans express a cre::gfp reporter gene in which the CRE (CAMP response element) DNA sequence is fused upstream of the gene encoding GFP.²⁷ CREB (CRE binding protein) is a transcription factor that, when activated, binds to the CRE sequence and induces transcription of genes downstream of CRE. In the CRE-GFP animal, GFP fluorescence is detected in cells in which activated CREB binds to CRE. Under normal conditions, GFP is seen in head mesodermal cells, some pharyngeal cells, and excretory glands. GFP is only rarely and weakly detected in neurons under normal conditions.²⁷ However, it was determined that GFP fluorescence can be induced in 4 cholinergic head neurons (named SIA) and regulated by dopamine (FIG. 2b).^{25,26} SIA neurons receive synaptic input from the dopaminergic CEP neurons. Basal stimulation of the G-protein-coupled D2-like dopamine receptors expressed on SIA neurons was demonstrated to inhibit CREB activation. This CREB inhibition due to upstream dopamine receptor stimulation is associated with minimal or no GFP expression in SIA neurons. Conversely, with reduced or absent dopaminergic neuron activity, stimulation of dopamine receptors on SIA neurons is limited, CREB is activated, and GFP expression is observed in these neurons.^{25,26} For example, the cat-2(e1112) animal with deficient dopaminergic neurotransmission due to mutation in the cat-2 gene, which encodes the enzyme tyrosine hydroxylase required for dopamine synthesis, exhibits frequent spontaneous GFP fluorescence in SIA neurons. Thus, minimal or absent GFP fluorescence in SIA neurons indicates intact dopaminergic neuron signalling, whereas spontaneous GFP fluorescence in SIA neurons indicates impairment of dopaminergic neuron signalling (FIG. 2b). Using this system, it was tested whether the coiling phenotype of A30P α-synuclein animals is associated with impaired dopaminergic neuron signalling. Four-day-old young adult animals expressing A30P α-synuclein in dopaminergic neurons exhibited spontaneous GFP fluorescence in SIA neurons, similar to cat-2(e1112) animals (FIG. 2c). In contrast, control animals with the cre::gfp reporter gene, but without α-synuclein, displayed minimal spontaneous GFP signal in SIA neurons.

If coiling of A30P α-synuclein C. elegans occurs due to impaired dopaminergic neuron signaling, one predicts that activation of dopamine receptors with exogenous dopamine would reduce the phenotype. The effect of dopamine treatment on motor behavior was examined and found that coiling of the animals decreased with treatment in a dose-dependent manner (FIG. 2d). Moreover, impairment of dopaminergic neuron signaling in animals not expressing α-synuclein was induced by treating them with raclopride, a D2 dopamine receptor antagonist. It was found that D2 receptor blockade in these non-α-synuclein expressing animals recapitulated the coiling behaviour of transgenic α-synuclein C. elegans (FIG. 2d). The results from these pharmacological experiments imply that coiling can result from dopaminergic synaptic dysfunction, whether due to postsynaptic dopamine receptor antagonism or to impaired presynaptic signaling associated with α-synuclein expression.

Although coiling has been previously reported for several unc C. elegans strains, common molecular pathways for this motor phenotype have not been determined. To identify such pathways, strains that coil were extracted from a database of motor behaviours of 300 different C. elegans mutant strains.¹¹ A gene ontology (GO) enrichment analysis of the genes mutated in the C. elegans strains with the coiler phenotype was then performed to examine for the over-representation of genes associated with specific biological processes (Table 1). Among the most highly over-represented gene classes associated with coiling were those related to dopamine receptor signaling, dopamine-related behaviour (i.e., response to food²⁶), and dopamine metabolism (i.e., dopamine/catechol-containing metabolic/biosynthetic process) (FIG. 2e). These results taken together suggest that, prior to causing loss of dopamine neurons, α-synuclein accumulation in dopaminergic neurons leads to impaired presynaptic signalling, which results in the coiling behavior.

TABLE 1
Gene ontology (GO) enrichment analysis of C. elegans strains with coiler phenotype.
Biological processes are listed if over-represented by ≥100-fold within the GO
over-representation analysis of biological processes using PANTHER “GO biological
process complete” (pantherdb.org/geneListAnalysis.do). C. elegans genome was used
as the reference list. Biological processes with ≥100-fold enrichment were ranked according
to −log₁₀ of the false discovery rate (FDR).

| | Gene Ontology | # of Genes | # of Genes | −log₁₀ |
|---|---|---|---|---|
| Biological Process | Term | from Input | Expected | (FDR) |
| Dopamine receptor signaling pathway | (GO: 0007212) | 8 | 0.07 | 11.266 |
| Response to food | (GO: 0032094) | 5 | 0.04 | 6.669586 |
| Calcium ion import across plasma membrane | (GO: 0098703) | 3 | 0.02 | 3.761954 |
| Calcium ion import into cytosol | (GO: 1902656) | 3 | 0.02 | 3.759451 |
| Adrenergic receptor signaling pathway | (GO: 0071875) | 3 | 0.02 | 3.756962 |
| Adenylate cyclase-activating adrenergic | (GO: 0071880) | 3 | 0.02 | 3.754487 |
| receptor signaling pathway | | | | |
| Catechol-containing compound | (GO: 0009712) | 3 | 0.03 | 3.605548 |
| metabolic process | | | | |
| Catechol-containing compound | (GO: 0009713) | 3 | 0.03 | 3.603801 |
| biosynthetic process | | | | |
| Dopamine metabolic process | (GO: 0042417) | 3 | 0.03 | 3.60206 |
| Catecholamine biosynthetic process | (GO: 0042423) | 3 | 0.03 | 3.598599 |
| Catecholamine metabolic process | (GO: 0006584) | 3 | 0.03 | 3.596879 |
| Synaptic target recognition | (GO: 0008039) | 2 | 0.01 | 2.31158 |
| Serotonin biosynthetic process | (GO: 0042427) | 2 | 0.01 | 2.310691 |
| Indole-containing compound | (GO: 0042435) | 2 | 0.01 | 2.308919 |
| biosynthetic process | | | | |
| Dopamine biosynthetic process | (GO: 0006585) | 2 | 0.01 | 2.307153 |
| from tyrosine | | | | |
| Primary amino compound | (GO: 1901162) | 2 | 0.01 | 2.305395 |
| biosynthetic process | | | | |
| Adenylate cyclase-activating | (GO: 0007191) | 2 | 0.01 | 2.303644 |
| dopamine receptor signaling pathway | | | | |
| Positive regulation of synaptic | (GO: 0032230) | 2 | 0.02 | 2.155523 |
| transmission, gabaergic | | | | |
| Regulation of synaptic vesicle | (GO: 2000300) | 2 | 0.02 | 2.154282 |
| exocytosis | | | | |
| Serotonin metabolic process | (GO: 0042428) | 2 | 0.02 | 2.152427 |
| Nonassociative learning | (GO: 0046958) | 2 | 0.02 | 2.151195 |
| Habituation | (GO: 0046959) | 2 | 0.02 | 2.149354 |
| Primary amino compound | (GO: 1901160) | 2 | 0.02 | 2.14813 |
| metabolic process | | | | |

Alterations in Protein Control Pathways that Regulate α-Synuclein Accumulation Affect Motor Function

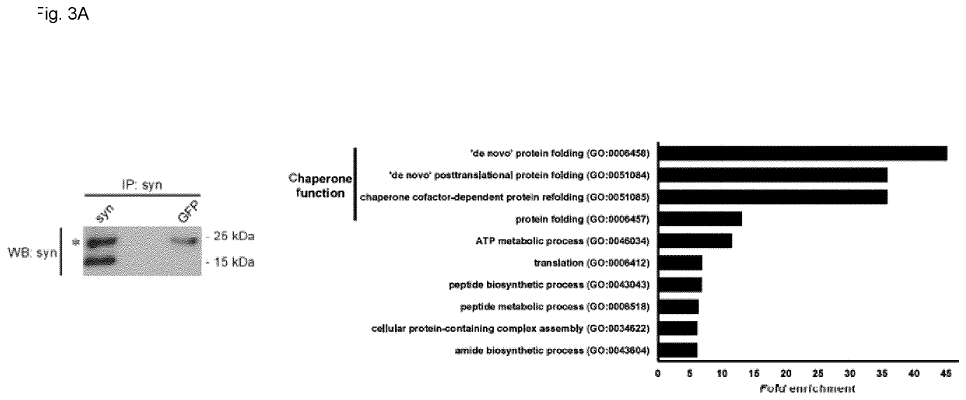

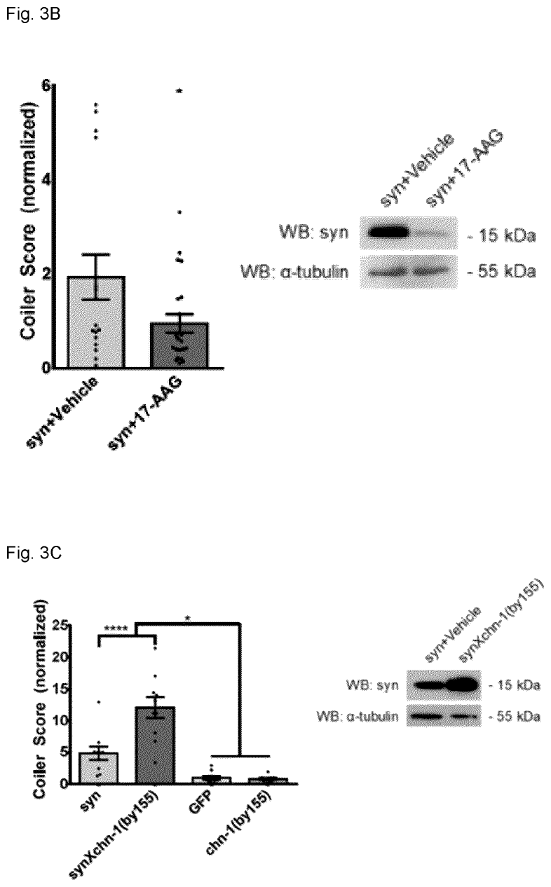

Abnormal accumulation of α-synuclein protein can occur in humans with PD due to defective cellular mechanisms responsible for protein quality control.²⁸ To determine which systems may be regulating α-synuclein accumulation in C. elegans, we identified proteins that bind to α-synuclein were identified. Immunoprecipitations were performed from α-synuclein or GFP C. elegans lysates with anti-α-synuclein antibodies (FIG. 3a), and co-immunoprecipitated proteins were then identified by mass spectrometry. After excluding non-specific binding proteins that were present in both α-synuclein and GFP conditions, a total of 131 C. elegans proteins were identified as interacting with α-synuclein. These proteins were analysed using GO enrichment analysis (Table 2). Among the α-synuclein interactors, we it was found that the most highly over-represented proteins were involved in chaperone function (FIG. 3a).

TABLE 2
Gene ontology (GO) enrichment analysis of proteins co-immunoprecipitated with α-synuclein
from C. elegans lysates. The biological processes are ranked by fold enrichment within the
GO over-representation analysis of biological processes using PANTHER “GO biological process
complete” (pantherdb.org/geneListAnalysis.do). The C. elegans genome was used as the
reference list. Only biological processes with a false discovery rate (FDR) ≤0.05 are listed.

| | Gene Ontology | # of Genes | # of Genes | Fold |
|---|---|---|---|---|
| Biological Process | Term | from Input | Expected | Enrichment |
| ‘De novo’ protein folding | (GO: 0006458) | 4 | 0.09 | 45.17 |
| Chaperone cofactor-dependent protein | (GO: 0051085) | 3 | 0.08 | 35.87 |
| refolding | | | | |
| ‘De novo’ posttranslational protein | (GO: 0051084) | 3 | 0.08 | 35.87 |
| folding | | | | |
| Protein folding | (GO: 0006457) | 6 | 0.46 | 13.11 |
| ATP metabolic process | (GO: 0046034) | 6 | 0.52 | 11.62 |
| Translation | (GO: 0006412) | 9 | 1.29 | 6.98 |
| Peptide biosynthetic process | (GO: 0043043) | 9 | 1.31 | 6.85 |
| Peptide metabolic process | (GO: 0006518) | 10 | 1.57 | 6.35 |
| Cellular protein-containing complex | (GO: 0034622) | 9 | 1.45 | 6.22 |
| assembly | | | | |
| Amide biosynthetic process | (GO: 0043604) | 9 | 1.46 | 6.18 |
| Cytoskeleton organization | (GO: 0007010) | 9 | 1.54 | 5.84 |
| Cellular amide metabolic process | (GO: 0043603) | 10 | 1.97 | 5.08 |
| Protein-containing complex assembly | (GO: 0065003) | 9 | 1.79 | 5.04 |
| Organelle organization | (GO: 0006996) | 17 | 5.39 | 3.15 |
| Cellular component organization or | (GO: 0071840) | 24 | 8.43 | 2.85 |
| biogenesis | | | | |
| Cellular component organization | (GO: 0016043) | 22 | 7.83 | 2.81 |

The chaperone system is composed of chaperone proteins, such as HSP70 (heat shock protein 70), and co-chaperones. Together, these proteins are integral to maintaining cellular protein quality control by folding newly translated polypeptides into their native conformation, refolding misfolded proteins to prevent their aggregation, and targeting proteins to degradation pathways, such as the autophagy-lysosomal system or ubiquitin-proteasome system, when refolding is unsuccessful. From the above in vitro findings, it was predicted that perturbations to the chaperone system in vivo would affect coiling of the transgenic α-synuclein C. elegans. To test this prediction, we treated the animals were treated with the geldanamycin analogue, 17-(allylamino)-17-demethoxygeldanamycin (17-AAG). Geldanamycin and its analogues are small molecules previously shown in cultured human cells to upregulate the chaperone system by increasing expression of HSP70 and to reduce α-synuclein accumulation and associated cell death.²⁹⁻³² We It was found that 17-AAG treatment resulted in lower α-synuclein protein levels and reduced coiling by 51% compared with vehicle control (FIG. 3b). To examine the effect of downregulating the chaperone system, we crossed the transgenic α-synuclein C. elegans were crossed with the chn-1(by155) animal to generate an animal lacking functional CHIP (carboxyl-terminus of HSP70 interacting protein). CHIP is a co-chaperone of HSP70 that assists in refolding of proteins and directing proteins, such as α-synuclein, for degradation.³³ The chn-1(by155) animals without α-synuclein exhibited limited coiling, similar to the transgenic control GFP animals. In contrast, the chn-1(by155) animals with α-synuclein demonstrated a 47% increase in coiling, which was greater than the coiling behaviour of the α-synuclein alone animals (FIG. 3c). The more severe coiling was associated with higher levels of α-synuclein protein. Taken together, these results demonstrate that decreases or increases in α-synuclein C. elegans coiling occur with perturbations in protein quality control systems previously shown to inhibit or enhance cytotoxicity induced by α-synuclein, respectively. These findings imply that a reduction in coiling of these animals may identify interventions, such as gene mutations or small molecules, that inhibit α-synuclein cytotoxicity in vivo.

Methods are described in Example 6.

### Example 5

### Compounds Identified in a Combined in Silico, In Vitro, and In Vivo Screen Reduce α-Synuclein Oligomers in Mammalian Models

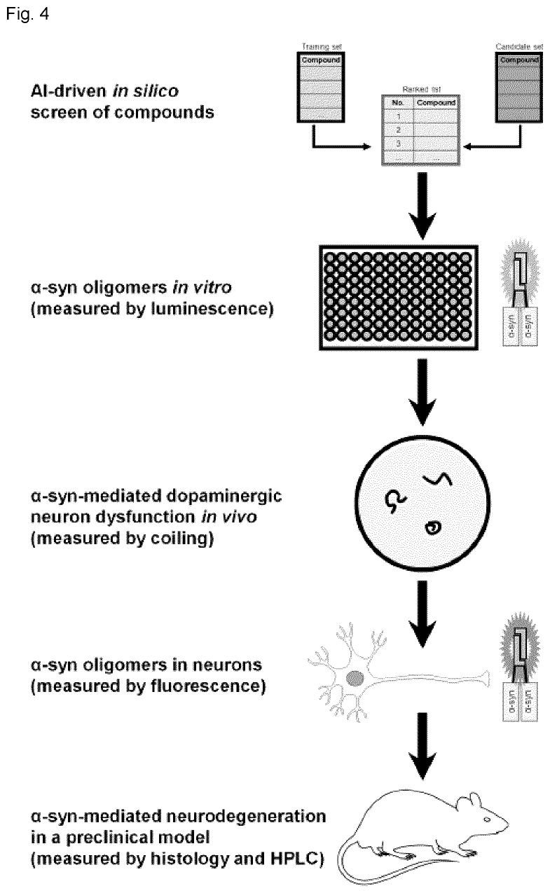

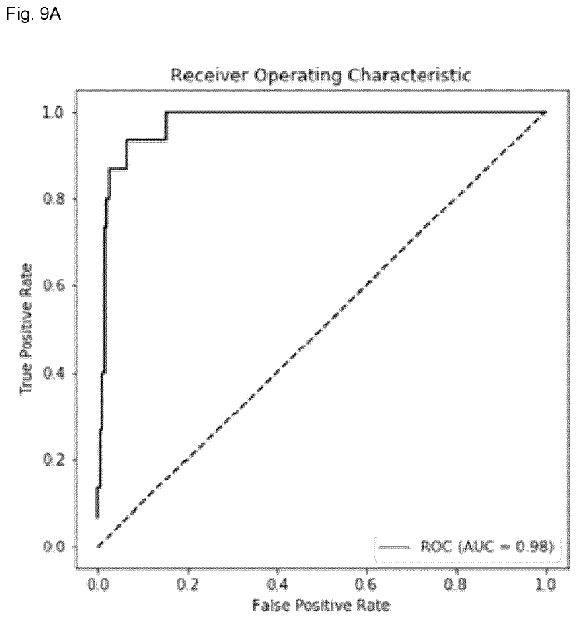

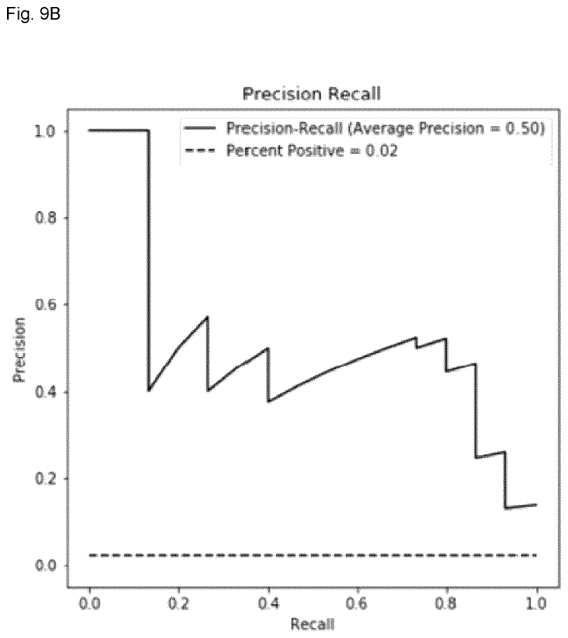

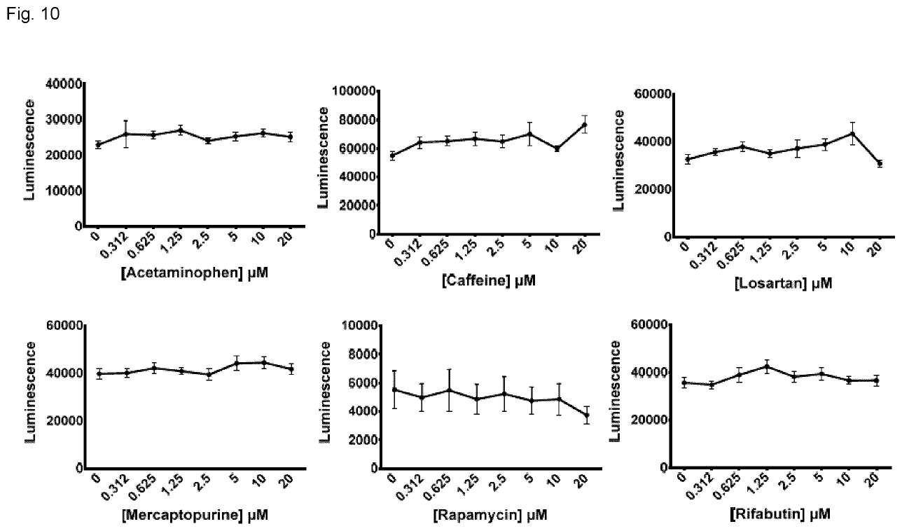

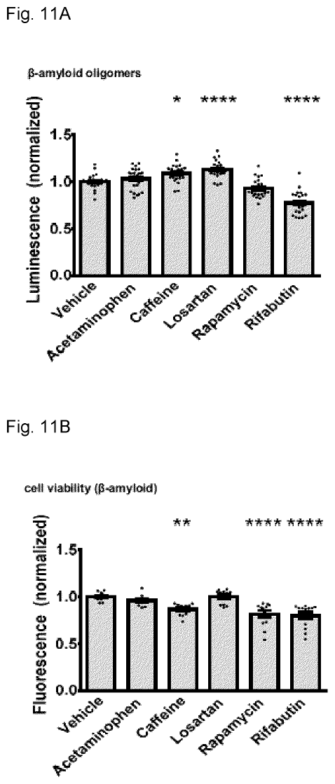

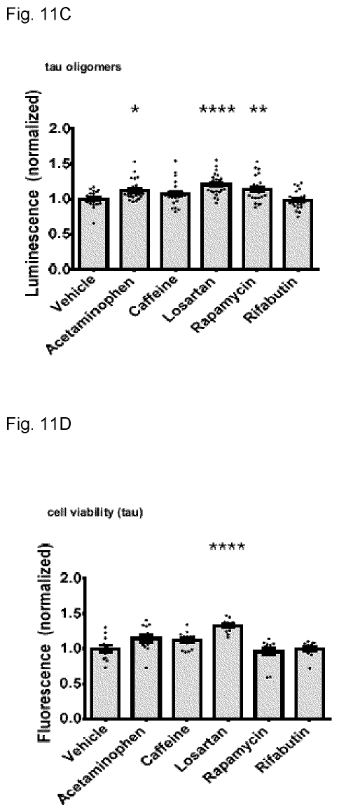

The application of this C. elegans model was explored by incorporating it into a screening strategy to identify compounds that inhibit α-synuclein aggregation and cytotoxicity (FIG. 4). In this strategy, drugs already approved for human use for treatment of diseases other than PD and thus have the potential for repurposing or repositioning were investigated.³⁴ First, an AI-driven in silico method to predict compounds with a high likelihood of inhibiting aggregation of α-synuclein into oligomers was used. IBM Watson for Drug Discovery Predictive Analytics is a cloud-based computing platform that employs natural language processing to extract domain-specific text features from the abstracts of published manuscripts.^{35,36} It creates a semantic model from a set of known entities that share a common property of interest and then applies the model to rank a set of candidate entities according to their similarity to the known set using a graph diffusion algorithm.^{35,36} For the screen described herein, the known entities were 15 small molecules previously shown to reduce α-synuclein oligomers in cell or animal models (Table 3),^{29-31,37-43} and the candidate entities were 620 compounds currently prescribed for treatment of various human diseases and tracked in health care administrative databases of prescription drug utilization (Table 4). The model's performance was assessed with leave-one-out (LOO) cross-validation in which the graph diffusion algorithm was applied 15 times, each time with one entity removed from the known set and added to the candidate set. It was found that 13 of the 15 known entities ranked in the top 30 when added to the 620 candidate entities, and the ranking scores of the known entities were significantly greater than those of the rest of the candidates (one-tailed Wilcoxon Sum Rank test, p=7.02×10⁻¹⁰) (Table 4). The precision-recall curve had an average precision of 0.5 which was always above precision due to chance (percent positive=0.02) (FIG. 9a). In addition, the area under the Receiver-Operating Characteristic curve (AUC) was 0.975 (FIG. 9b), further suggesting high predictive performance of the model (AUC=1 corresponds to a perfectly predictive model). Using this model, the 620 candidate compounds were ranked by similarity to the known entities that reduce α-synuclein oligomers (Table 4).

TABLE 3
Known entities are compounds previously shown to reduce
α-synuclein oligomers in cell or animal models in hypothesis-
driven studies (see references). The corresponding number
of abstracts of published abstracts identified and used
by IBM Watson for Drug Discovery Predictive Analytics for
each drug on the date of ranking is provided.

| Drug Name | # of Abstracts |
|---|---|
| 17-AAG²⁹⁻³¹ | 816 |
| AICAR³⁹ | 446 |
| Anle183b⁴² | 5 |
| Baicalein⁴⁰ | 1000 |
| Curcumin⁴¹ | 1000 |
| Cyclosporine³⁰ | 1000 |
| Geldanamycin³⁷ | 1000 |
| Isorhynchophylline⁴³ | 51 |
| Myricetin⁴¹ | 1000 |
| PD169316³⁰ | 176 |
| Resveratrol³⁹ | 1687 |
| Rosmarinic acid⁴¹ | 1021 |
| SB239063³⁰ | 122 |
| Tacrolimus³³ | 1000 |
| TBBz³⁰ | 19 |

TABLE 4
In silico ranking of compounds by IBM Watson for Drug Discovery
Predictive Analytics based on their likelihood of inhibiting
α-synuclein aggregation. Candidate entities (N = 620)
are oral medications currently prescribed for treatment of various
human diseases and listed in the Ontario Drug Benefit Program
formulary (formulary.health.gov.on.ca/formulary/). Known entities
(N = 15) are in bold. Ranking is shown for each of the
known entities when removed from the known set and added to
the candidate set during leave-one-out validation analysis.
Compounds highlighted in grey were selected for testing in vitro.

| | IBM Watson | |
|---|---|---|
| Drug Name | Ranking | Score |
| Curcumin | | 0.026572896 |
| Resveratrol | | 0.024891593 |
| Verapamil | 1 | 0.02391447 |
| Tretinoin | 2 | 0.023665065 |
| Caffeine | 3 | 0.02212228 |
| Dexamethasone | 4 | 0.021293124 |
| PD169316 | | 0.020369345 |
| SB239063 | | 0.020314941 |
| Sirolimus | 5 | 0.019989299 |
| Indomethacin | 6 | 0.019799477 |
| Etoposide | 7 | 0.019230978 |
| Baicalein | | 0.018408176 |
| Geldanamycin | | 0.018190702 |
| Estradiol | 8 | 0.018186088 |
| Celecoxib | 9 | 0.017885722 |
| Framycetin | 10 | 0.016386577 |
| 17-AAG | | 0.013898701 |
| Isorhynchophylline | | 0.01359914 |
| TBBz | | 0.011656517 |
| Myricetin | | 0.011613904 |
| Rosmarinic acid | | 0.011587948 |
| Sulindac | 11 | 0.01155053 |
| Tamoxifen | 12 | 0.01073828 |
| Cyclosporine | | 0.010528345 |
| Piroxicam | 13 | 0.010327113 |
| Minocycline | 14 | 0.01029561 |
| Ketoconazole | 15 | 0.010211005 |
| Alitretinoin | 16 | 0.009433155 |
| AICAR | | 0.009317443 |
| Lovastatin | 17 | 0.009266705 |
| Erlotinib | 18 | 0.009241609 |
| Ascorbic acid | 19 | 0.009162541 |
| Flurbiprofen | 20 | 0.008901568 |
| Tetracycline | 21 | 0.00810332 |
| Naproxen | 22 | 0.007928475 |
| Propranolol | 23 | 0.007212216 |
| Temozolomide | 24 | 0.006998555 |
| Sennosides | 25 | 0.006921414 |
| Imatinib | 26 | 0.006875301 |
| Allopurinol | 27 | 0.006825175 |
| Galantamine | 28 | 0.00679403 |
| Rifabutin | 29 | 0.006793076 |
| Calcitriol | 30 | 0.006743945 |
| Vincristine | 31 | 0.006730485 |
| Pioglitazone | 32 | 0.00670246 |
| Mercaptopurine | 33 | 0.006681412 |
| Melphalan | 34 | 0.006625598 |
| Losartan | 35 | 0.006591429 |
| Ibuprofen | 36 | 0.006536924 |
| Theophylline | 37 | 0.006147827 |
| Acetaminophen | 38 | 0.00607345 |
| Hydrocortisone | 39 | 0.005921451 |
| Pseudoephedrine | 40 | 0.005077118 |
| Collagenase | 41 | 0.004775753 |
| Auranofin | 42 | 0.004727561 |
| Pentoxifylline | 43 | 0.004670313 |
| Miconazole | 44 | 0.004667356 |
| Polyethylene glycol | 45 | 0.004591185 |
| Anle183b | | 0.004580211 |
| Ferrous sulfate | 46 | 0.004574955 |

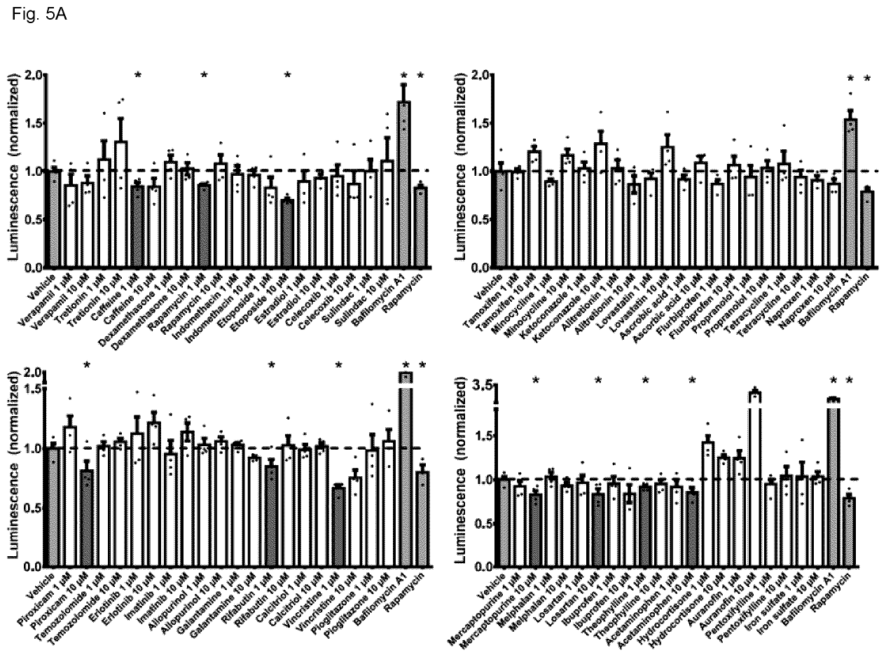

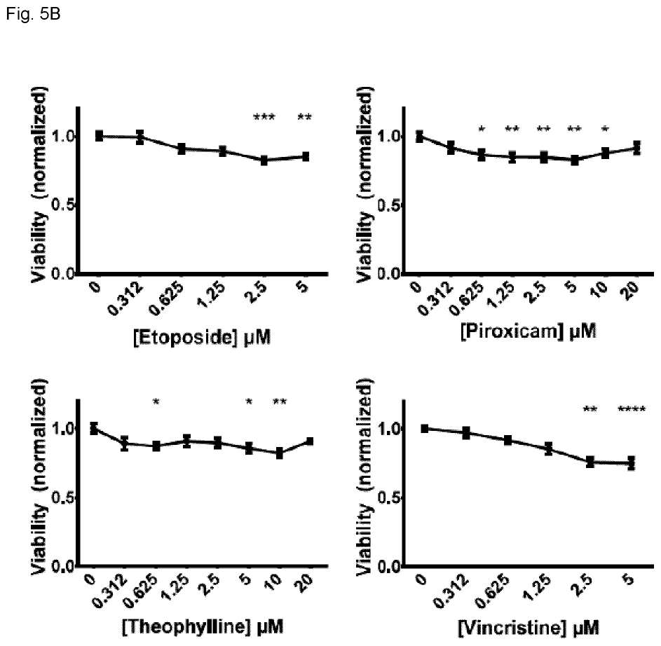

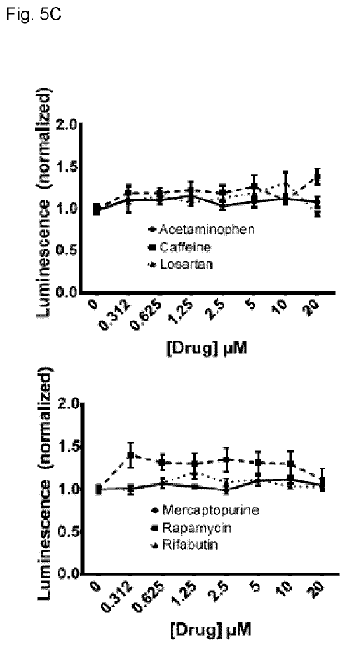

Next, 40 compounds ranked highly in silico based on predictions of their ability to reduce α-synuclein oligomers were tested in vitro (Table 5). A bioluminescent protein-fragment complementation assay was used in which human H4 neuroglioma cells co-express human α-synuclein tagged with the N- or C-terminal half of Gaussia princeps luciferase (FIG. 4).^{29-31,33,44} Reconstitution of a complete luciferase molecule from the fragments occurs upon α-synuclein oligomerization and hence luciferase activity provides a surrogate measure of α-synuclein oligomer levels. For controls in this assay, vehicle (i.e., DMSO), rapamycin, and bafilomycin A were used. Rapamycin, or sirolimus, is a small molecule that activates autophagy by inhibiting mTOR (mammalian target of rapamycin). In this assay, treatment of the cells with the autophagy inhibitor bafilomycin A1 resulted in an increase in α-synuclein oligomers, whereas treatment with rapamycin caused a decrease in α-synuclein oligomer levels (FIG. 5a). 9 additional compounds were identified as causing a statistically significant decrease in luciferase activity compared with vehicle alone: acetaminophen (14% decrease), caffeine (16% decrease), etoposide (30% decrease), losartan (16% decrease), mercaptopurine (17% decrease), piroxicam (19% decrease), rifabutin (15% decrease), theophylline (8% decrease), and vincristine (33% decrease) (FIG. 5a). It is possible that lower absolute α-synuclein oligomer levels would be measured if a compound caused cell death and thereby reduced the number of surviving α-synuclein-expressing cells. To test this possibility, cells expressing α-synuclein were treated with each of the compounds and a cell permeable, resazurin-based assay was used to test for cell viability. It was found that treatment with etoposide, piroxicam, theophylline, or vincristine, but not with the other compounds, reduced cell survival by >15% (FIG. 5b). Thus, these four compounds were excluded from further study since their effects on α-synuclein oligomer measures may be secondary to cell death. Moreover, small molecules with significant cell toxicity are unlikely to be amenable to repurposing for a neurodegenerative disease. None of the remaining compounds reduced the activity of full-length luciferase when cells expressing luciferase alone were treated (FIG. 5c). It was therefore inferred that the compounds reduced the absolute amount of α-synuclein oligomers in this assay.

TABLE 5
Forty compounds ranked highly by an Al-driven in silico platform based on its predictions
of their ability to reduce α-synuclein oligomers were tested in vitro.

| | | Canonical Drug | Potential Links to α-synuclein |
|---|---|---|---|
| Ranking | Drug Name | Function/Indication | and PD |
| 1 | Verapamil | Voltage-dependent calcium | Calcium channel blockers within its |
| | | channel blocker, various cardiac | family are associated with reduced |
| | | diseases | PD risk⁶² |
| 2 | Tretinoin | Acne medication, acute | Unknown |
| | | promyelocytic leukemia | |
| 3 | Caffeine | Inhibitor of adenosine A2A | Experimentally rescues rodent |
| | | receptor, commonly used | models of PD,⁶³ associated with |
| | | stimulant | reduced PD risk⁶⁴ |
| 4 | Dexamethasone | Corticosteroid, anti-inflammatory | Induces anti-inflammatory effect |
| | | | and reduces toxic α-synuclein |
| | | | aggregates⁶⁵ |
| 5 | Rapamycin | Immunosuppressant, post- | Induces autophagy to reduce α- |
| | | transplantation | synuclein aggregates in numerous |
| | | | models of PD⁴⁵ |
| 6 | Indomethacin | NSAID, COX inhibitor | Exhibits neuroprotective effect in |
| | | | mouse models of PD, similar to |
| | | | other anti-inflammatory drugs⁶⁶ |
| 7 | Etoposide | DNA topoisomerase inhibitor, | Unknown |
| | | anti-cancer | |
| 8 | Estradiol | Female sex hormone, prescribed | Exhibits neuroprotective effect on |
| | | for hormone replacement therapy | dopaminergic neurons exposed to |
| | | | MPP+ in vitro⁶⁷ |
| 9 | Celecoxib | NSAID, COX2 inhibitor, anti- | Rescues dopaminergic dysfunction |
| | | arthritic | in a rodent LPS-induced |
| | | | neurotoxicity model⁶⁸ |
| 11 | Sulindac | NSAID, COX inhibitor | Unknown |
| 12 | Tamoxifen | Estrogen receptor inhibitor, | Unknown |
| | | anti-breast cancer | |
| 13 | Piroxicam | NSAID, non-selective COX | Exhibited anti-fibrillogenic activity in |
| | | inhibitor | a drug screen of various NSAIDs⁶⁹ |
| 14 | Minocycline | Broad spectrum tetracycline- | Anti-inflammatory in MPTP mouse |
| | | class antibiotic | model⁷⁰ |
| 15 | Ketoconazole | Topical antimycotic | Rescued neurodegeneration in |
| | | | Drosophila model of α-synuclein |
| | | | expression⁷¹ |
| 16 | Alitretinoin | Eczema topical ointment | Unknown |
| 17 | Lovastatin | Inhibitor of de novo cholesterol | Reduces α-synuclein abundance in |
| | | synthesis, lowers blood | mouse models⁷² |
| | | cholesterol levels | |
| 18 | Erlotinib | EGFR inhibitor, chemotherapy | Unknown |
| 19 | Ascorbic acid | Common micronutrient, provides | Exhibits neuroprotective effect in |
| | | antioxidant function | Drosophila neurodegeneration⁷³ |
| 20 | Flurbiprofen | NSAID, possible COX inhibitor | Exhibited anti-fibrillogenic activity in |
| | | | a drug screen of various NSAIDs⁷⁴ |
| 21 | Tetracycline | Broad-spectrum antibiotic | Unknown |
| 22 | Naproxen | NSAID, nonselective COX | Exhibited weak anti-fibrillogenic |
| | | inhibitor | activity in a drug screen of various |
| | | | NSAIDs⁷⁴ |
| 23 | Propranolol | Anti-hypertensive drug, beta- | Increases abundance of SNCA |
| | | adrenergic receptor blocker | transcript⁷⁵ |
| 24 | Temozolomide | Alkylating agent, chemotherapy | Unknown |
| 26 | Imatinib | Bcr-Abl inhibitor, chemotherapy | Derivatives of this compound |
| | | | rescue neurodegeneration in an |
| | | | MPTP mouse model⁷⁶ |
| 27 | Allopurinol | Uric acid synthesis inhibitor, gout | Unknown |
| 28 | Galantamine | Acetylcholinesterase inhibitor, | Clinically used in PD dementia⁷⁷ |
| | | Alzheimer's disease | |
| 29 | Rifabutin | Antimycotic compound, | Unknown |
| | | tuberculosis | |
| 30 | Calcitriol | Increases blood calcium levels | Related compounds shown to |
| | | | reduce α-synuclein aggregation⁷⁸ |
| 31 | Vincristine | Microtubule destabilizing agent | Unknown |
| 32 | Pioglitazone | PPAR-γ inducer, diabetes | Neuroprotective in rodent models |
| | | | of PD^{79, 80} |
| 33 | Mercaptopurine | Purine analog, chemotherapy | Putative activator of |
| | | | neuroprotective factors⁸¹ |
| 34 | Melphalan | DNA alkylating agent, | Unknown |
| | | chemotherapy | |
| 35 | Losartan | Angiotensin receptor II | Exhibits neuroprotective effect in |
| | | antagonist, hypertension | cell and MPTP rodent model^{82, 83} |
| 36 | Ibuprofen | NSAID, COX inhibitor | Clinical correlation with reduced PD |
| | | | risk⁸⁴ |
| 37 | Theophylline | Bronchodilator | Unknown |
| 38 | Acetaminophen | Analgesic, anti-pyretic | Rescues C. elegans models of |
| | | | dopaminergic neurodegeneration⁸⁵ |
| 39 | Hydrocortisone | Steroid, anti-inflammatory | Unknown |
| 42 | Auranofin | Anti-inflammatory, arthritis | Exhibits neuroprotective effect by |
| | | | reducing inflammation⁸⁶ |
| 43 | Pentoxifylline | Nonselective phosphodiesterase | Rescues 6-OHDA rat model of |
| | | inhibitor | PD⁸⁷ |
| 46 | Iron sulfate | Iron nutrient supplement | Shows interaction with α-synuclein, |
| | | | possibly inducing sequestration⁸⁸ |

These highly ranked compounds were excluded from in vitro testing for the following reasons: framycetin (ranking 10) and miconazole (ranking 44) are topical medications (not oral medications), sennosides (ranking 25) and polyethylene glycol (ranking 45) are laxatives, pseudoephedrine (ranking 40) is a controlled substance with limited access, and collagenase (ranking 41) is an enzyme (not a small molecule).

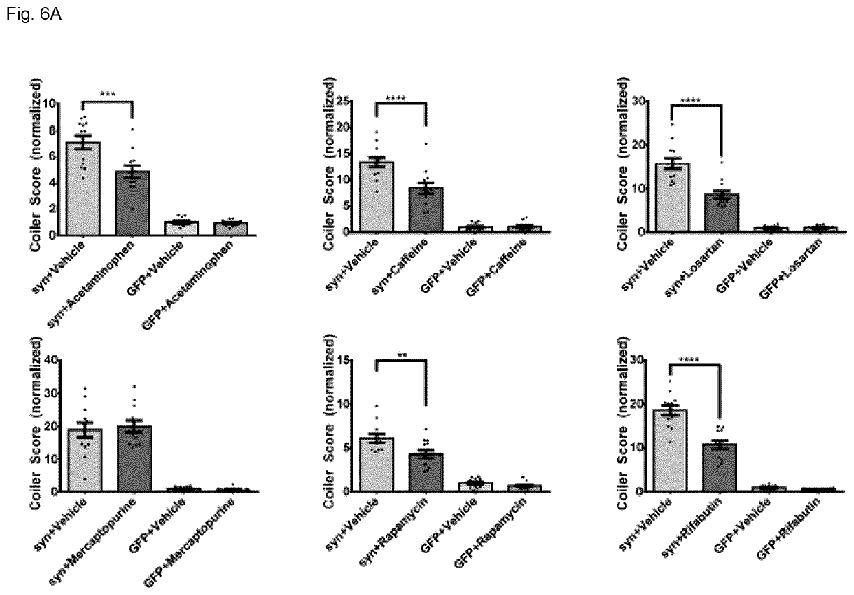

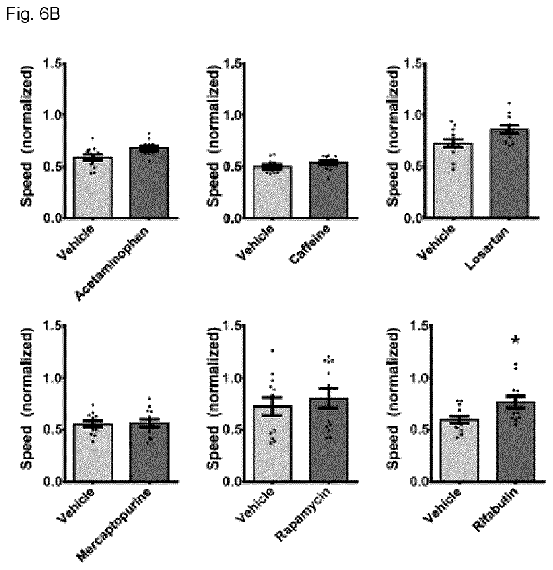

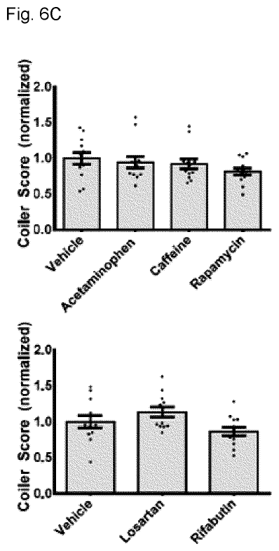

The transgenic α-synuclein C. elegans model described herein was then applied to test in vivo the 6 compounds that lowered α-synuclein oligomers but were not cytotoxic in vitro: acetaminophen, caffeine, losartan, mercaptopurine, rapamycin, and rifabutin. α-synuclein C. elegans or control GFP C. elegans were treated with each compound or vehicle control. It was found that treatment with 5 of the compounds caused a statistically significant reduction in coiling of α-synuclein C. elegans compared with vehicle alone: acetaminophen (32% decrease), caffeine (37% decrease), losartan (45% decrease), rapamycin (30% decrease), or rifabutin (42% decrease) (FIG. 6a). In contrast, mercaptopurine had no effect on the coiling behaviour of these animals. The minimal degree of coiling exhibited by control GFP C. elegans was unaffected by treatment with any of the compounds (FIG. 6a). Locomotor speed was also measured since reduced speed due to treatment could be a confound by decreasing the frequency of coiling events. It was found that treatment of these animals with each of the compounds did not decrease speed (FIG. 6b). To test whether the effects of acetaminophen, caffeine, losartan, rapamycin, and rifabutin were specific to coiling due to α-synuclein, unc-10(e102) animals which display a similar degree of coiling as α-synuclein animals, but due to mutation in an ortholog of human RIMS1 were treated, (FIG. 1d).¹⁴ It was found that none of these compounds affected the coiling behaviour of unc-10(e102) animals (FIG. 6c). Therefore, it was concluded that acetaminophen, caffeine, losartan, rapamycin, and rifabutin can each partially rescue the early dopamine-mediated motor impairment induced by α-synuclein in C. elegans.

### Validation of Screen Hits in Mammalian Models of α-Synuclein-Mediated Neurodegnerataion

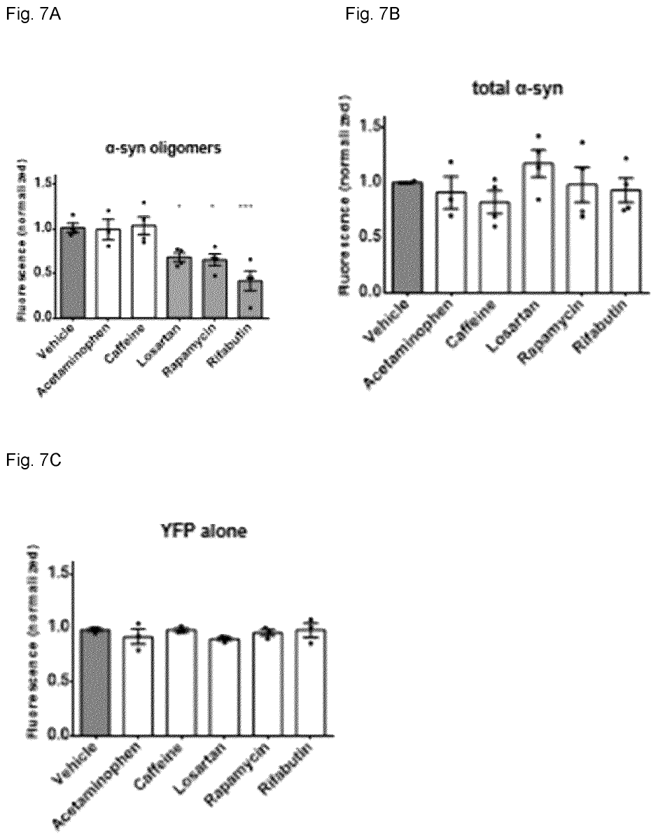

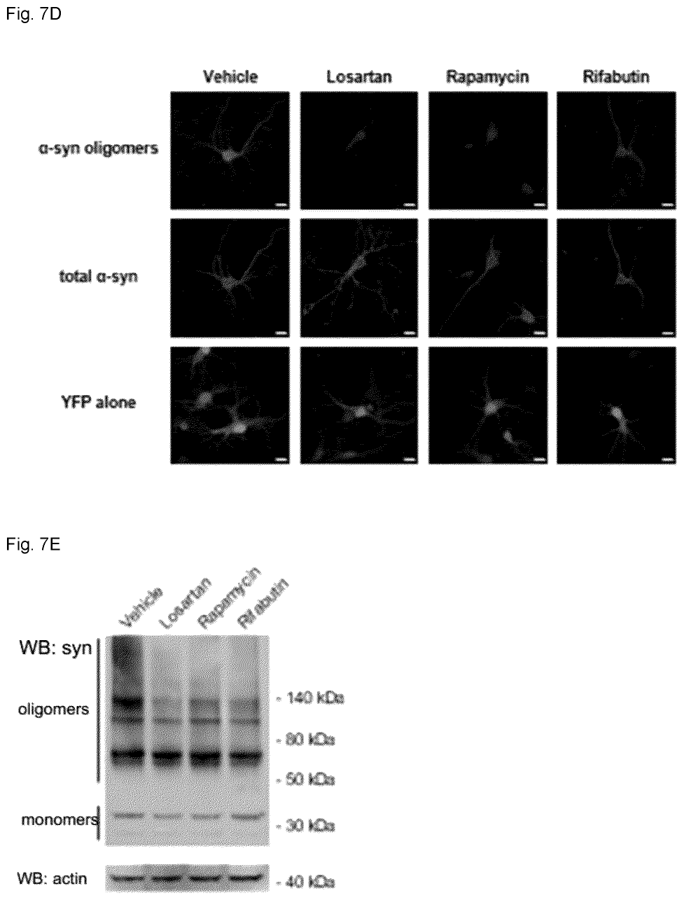

Finally, it was examined whether these 5 compounds that had effects in our invertebrate C. elegans model reduce α-synuclein oligomers and α-synuclein-mediated dopaminergic neurodegeneration in mammalian systems. Specifically, adeno-associated viral vectors (AAV) were used to co-express human α-synuclein tagged with the N- or C-terminal half of Venus yellow fluorescent protein (YFP) in rat cortical neurons. Similar to the luciferase protein-fragment complementation assay described above, reconstitution of a complete YFP molecule from the split YFP halves occurs upon α-synuclein oligomerization and thus spontaneous fluorescence provides an estimated measure of α-synuclein oligomer levels (FIG. 4).^{21,45,46,58,62} These neurons were treated with acetaminophen, caffeine, losartan, rapamycin, or rifabutin. 3 compounds were found to cause a statistically significant decrease in fluorescence compared with vehicle alone: losartan (33% decrease), rapamycin (36% decrease), and rifabutin (60% decrease) (FIG. 7a,d); this was associated with a reduction in α-synuclein oligomers as detected by immunoblotting (FIG. 7e). In contrast, none of the compounds reduced total α-synuclein levels (FIG. 7b,d) or YFP fluorescence when neurons expressing full-length YFP alone were treated (FIG. 7c,d). Thus, this screening strategy identified 3 compounds that reduce α-synuclein oligomer levels in mammalian neurons: rapamycin, which has previously demonstrated efficacy in an in vivo α-synuclein toxicity model of PD,⁵⁹ losartan, which has previously shown neuroprotective potential but not via an α-synuclein-mediated mechanism^{63,64} and rifabutin, which has not yet been explored as a treatment for neurodegenerative diseases.

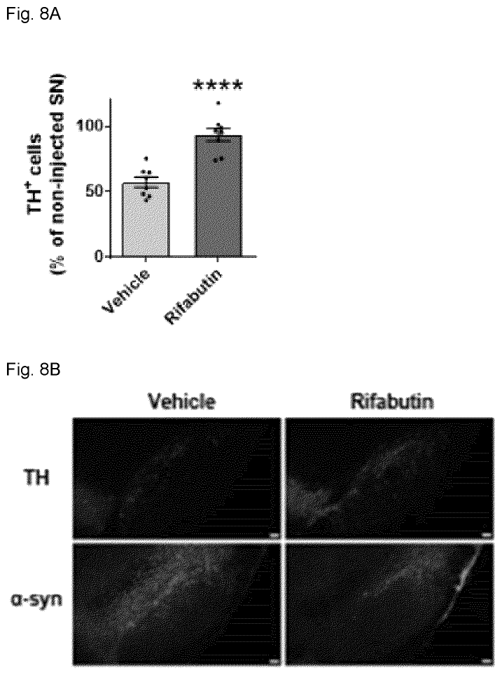

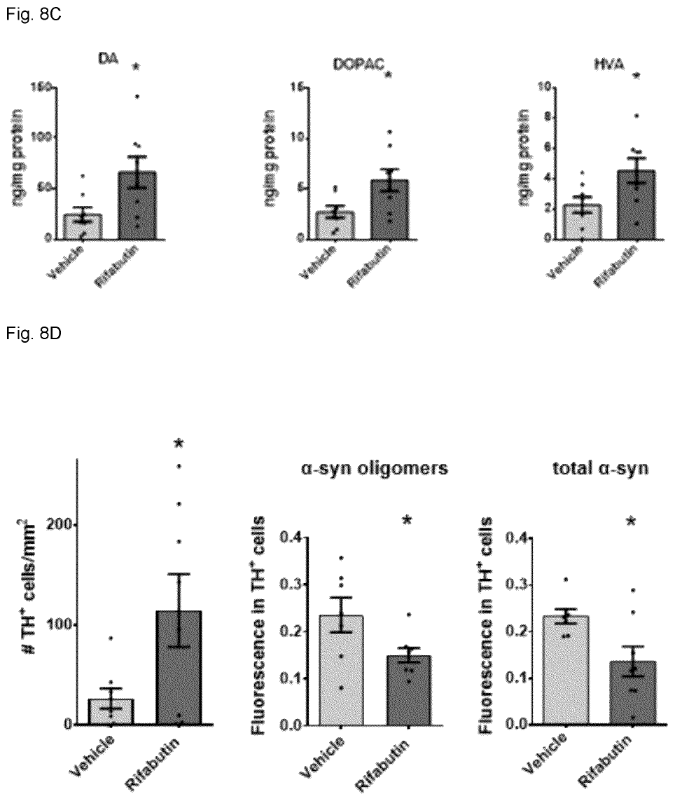

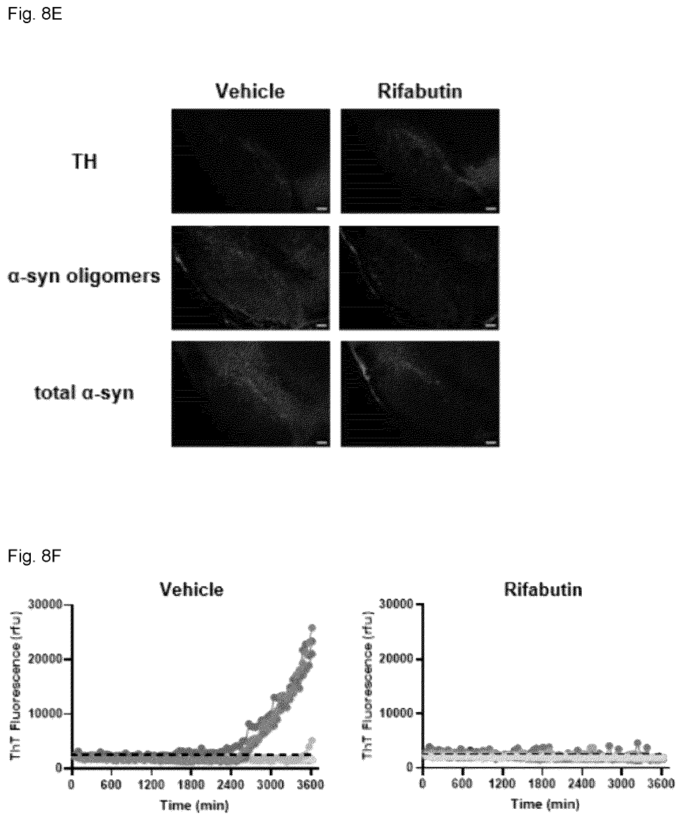

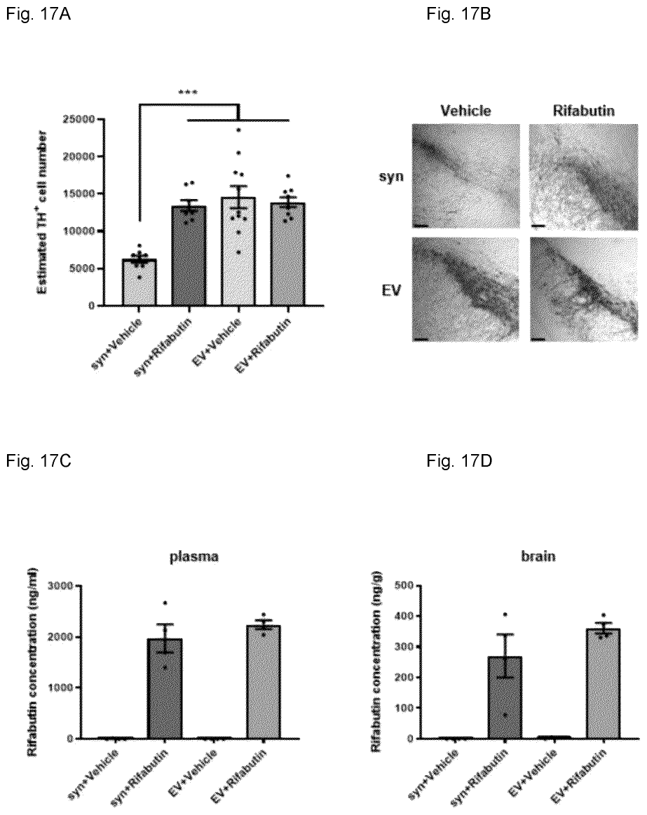

Rifabutin, a member of the ansamycin antibiotic family, is approved and currently used for chronic prophylactic treatment of disseminated Mycobacterium avium complex disease in people with HIV. Although rifabutin is a relatively large molecule (847 Da), it is highly lipophilic and has been reported to demonstrate moderate penetration of the blood-brain barrier.⁶⁵ To test the efficacy of rifabutin in reducing α-synuclein-medicated dopaminergic neurodegeneration in a mammalian model in vivo, we used an AAV-based rat model in which a unilateral stereotactic injection of virus results in expression of human mutant A53 T α-synuclein in the dopaminergic neurons of the SN^{28,66} the most prominently affected brain region in PD. The model consistently shows loss of SN dopaminergic neurons and loss of markers of dopamine function in the striatum (the primary brain region that receives projections from the SN dopaminergic neurons) including dopamine and its metabolites. Rats were randomized to receive vehicle or rifabutin at a dose equivalent to that used in humans.⁶⁷ Previous studies found no significant toxicological effects in rats at this dose⁶⁸. Consistent with this, no statistically significant differences in average body weights of animals treated with vehicle versus rifabutin over the course of the study were found. Animals treated with rifabutin demonstrated less dopaminergic cell death in the SN (FIG. 8a,b). This corresponded with higher levels of dopamine and its metabolites, 3,4-dihyroxyphenylacetic acid (DOPAC) and homovanillic acid (HVA), in the striatum of rifabutin treated animals when compared with vehicle treated animals (FIG. 8c). The AAV-A53 T model consistently shows loss of dopanminergic neurons in the injected SN, which we confirmed in this study using two separate methods. First, we demonstrated that the number of TH-positive cells in the AAV-A53 T injected SN of vehicle treated animals was only 57% compared to the non-injected SN (FIG. 8a, b). Second, we showed that the number of TH-positive cells in the AAV-A53 T injected SN of vehicle treated animals was significantly lower than the number of TH-positive cells in the SN of animals injected with an empty viral vector (EV) instead of AAV-A53 T (FIG. 17a,b). We confirmed brain delivery in our study by measuring steady-state drug concentrations in plasma and saline-perfused brain tissue from a subset of animals (FIG. 17c,d). The animals treated with rifabutin demonstrated less dopaminergic cell death in the SN compared to those treated with vehicle (FIG. 8a, b, d, FIG. 17a,b). This reduction in nigral neurodegeneration corresponded with higher levels of dopamine and its metabolites, 3,4-dihydoxyphenylacetic acid (DOPAC) and homovanillic acid (HVA), in the striatum (the primary brain region that receives projections from the SN dopaminergic neurons) (FIG. 8c). The levels of α-synuclein oligomers and total α-synuclein in the surviving dopaminergic SN neurons were lowered with rifabutin treatment (FIG. 8d, e). To determine whether rifabutin reduced disease-associated α-synuclein conformations, we applied a real-time quaking induced conversion (RT-QuIC) assay to detect seeding activity of α-synuclein from CSF (FIG. 8f). Indeed, 0% of CSF samples from rifabutin treated animals demonstrated positive seeding activity, whereas CSF from 4 of the 7 vehicle animals (57%) tested positive by RT-QuIC (Fisher's exact test, p=0.03).

Here a simple in vivo model that recapitulates fundamental features of PD, including dopaminergic neuron dysfunction due to α-synuclein and motor impairment that improves with dopamine treatment has been developed. Since C. elegans are not known to express a homolog of α-synuclein, the neuronal dysfunction induced by α-synuclein expression and the correlation of motor dysfunction, specifically coiling, with α-synuclein levels suggests a toxic gain-of-function, which is a proposed mechanism of α-synuclein-mediated neurodegeneration in humans.⁴⁹. In the model described herein, the coiler phenotype is caused by dopaminergic neuron dysfunction that precedes the loss of cell bodies and can be induced in non-α-synuclein expressing animals by pharmacological blockade of postsynaptic D2 receptors. Thus, it is inferred that coiling in this animal represents presynaptic dysfunction, further recapitulating the human disease. Coiling in the model described herein was not only an early phenotype but was also responsive to pharmacological or genetic manipulations that altered the chaperone system. Further, the method of measuring the coiling behaviour assessed multiple animals simultaneously,^{50,51} which could easily be scaled up and multiplexed, and thus there is the potential for developing a higher throughput system with this model.

While the simplicity of C. elegans makes it amenable to higher throughput analyses, it does not allow for all aspects of α-synuclein pathogenicity to be incorporated into a single model. The model described herein could be combined with recently described C. elegans that have indicators of cell-to-cell propagation to test compounds or pathways hypothesized to affect both dopaminergic dysfunction due to α-synuclein and its transmission. In this study, the C. elegans model described herein was used downstream of in silico and in vitro methods to identify compounds that inhibit neurodegeneration due to α-synuclein oligomers. The in silico methods, which incorporated AI components including natural language processing with machine learning,^{35,36} were used for the first time for this purpose. Compared to phenotypic screening in cells alone, the addition of upstream in silico predictions appeared to increase the yield of positive hits (e.g., 3 out of a 620 compound library in the screen described herein versus 2 out of a 1280 compound library in Moussaud et al.³⁰). Thus, these results support the potential utility of combining AI technologies with in vitro and in vivo testing to facilitate drug discovery for PD.

The validity of this approach for discovering compounds with the potential to target α-synuclein is supported by one of the candidates identified being rapamycin, a drug well known to enhance autophagy and thereby reduce α-synuclein accumulation.⁴⁵ Losartan, an angiotensin II receptor blocker, and rifabutin, an ansamycin antibiotic, were identified as candidates which, have not been previously explored as a potential treatment to target α-synuclein-mediated neurodegeneration. Rifabutin is a particularly attractive candidate as a treatment for PD or other neurodegenerative diseases because it can penetrate the blood brain barrier.

Methods are described in Example 6.

### Example 6

### Methods

C. elegans Strains

C. elegans were maintained at room temperature (21° C.) on nematode growth medium (NGM) agar plates with E. coli OP50 as a food source as previously described.⁵⁴ The following strains were obtained from Caenorhabditis Genetics Center (CGC; University of Minnesota, St. Paul, MN, USA): wild-type Bristol N2 animals and mutant strains BR2823 (by155), BZ555 (egls1[dat-1p::gfp]), CB102 (e102), CB587 (e587), CB933 (e245), and CB1112 (e1112). The following strains were obtained from National BioResource Project (NBRP; Japan): tmls903, tmls904, tmls905, tmls1082, tmls1083, and tmls1084. VN305, VN306, and VN307 were obtained by crossing YT2022 (tzls3[cre::gfp])²⁷ (kindly provided by Dr. Yoshishige Kimura, Kanagawa University of Human Services) with wild-type α-synuclein, A30P α-synuclein, and A53 T α-synuclein transgenic animals, respectively (kindly provided by Dr. Takeshi Iwatsubo, University of Tokyo).⁹ VN310 was obtained by backcrossing YT2022 with N2. VN162 was obtained by crossing YT2022 with CB1112. Control GFP animals were obtained by crossing VN310 with BZ555 and used as a control for ectopic protein expression in dopaminergic neurons, since GFP was expressed under the same dat-1 promoter as α-synuclein. VN306 was crossed with BR2823 to produce double mutants. VN306 was crossed with BZ555 to use GFP to track dopaminergic neuron loss over time.

C. elegans Locomotion

Animals were synchronized by isolating eggs using alkaline hypochlorite treatment and hatching overnight. Larvae were plated onto NGM agar plates with OP50 and grown for 4 days before recording locomotion. For individual C. elegans recordings, a single adult animal was randomly picked, washed for 1 to 2 minutes in M9 buffer, and then transferred to a fresh NGM plate without food for imaging. Video images were acquired (2 frames per second) for up to 6 minutes using a sCMOS camera (pco.edge, PCO) mounted on a stereomicroscope (Olympus MVX10 MacroView) controlled using the μManager software.⁵⁵ A total of 40 individual animals were imaged for each strain. For C. elegans population recordings, ten adult animals were randomly removed using an eyelash pick, washed for 1 minute in M9 buffer, and then transferred to a fresh NGM plate without food, with a ring of copper sulfate solution (150 mM) around the area of recording (a chemorepellent to keep the animals in the recording frame). Exploratory locomotion was recorded using a CCD camera (GX1920, Allied Vision) which was attached to a dissecting stereomicroscope (Leica M165C, 1.0× PlanApo lens). A Labview program captured video (2 frames per second) at 1936×1456 resolution. Using ImageJ, the displacement, perimeter, and area of each animal was calculated, from which a circularity value was computed using the following formula: circularity=4π(A/P²), where A is the area of the animal's body and P is the perimeter of the animal's body (both expressed in arbitrary pixel units). A cut-off for coiling was set at a circularity value of 0.6; an animal that had a circularity value >0.6 was considered to be coiling. Using MATLAB (Mathworks), various behavioural metrics including the coiler score were calculated, which is the percentage of frames with a circularity value >0.6. Speed was automatically computed from the ImageJ based WrmTrk plugin as the distance travelled by each animal (in pixels) over the amount of time that it was tracked (in seconds).56 Average speed was determined by calculating the weighted average of each tracked animal's speed within a single population of animals. On the day of the recording for dopamine or raclopride treatment, adult animals were washed for 1 minute in M9 buffer, treated in a solution containing either drug for 1 minute, and then placed onto fresh NGM plates without food, with a copper sulfate solution ring around the area of recording. For all other drug treatment experiments, larvae were first grown for 2 days after hatching and then treated on drug plates for 3 days prior to recording, since drug treatment of younger larvae resulted in abnormal development and unhealthy animals. Drug plates were prepared by adding each drug to LB medium with OP50 before spreading onto fresh NGM plates. The solvent used to dissolve each drug was used as vehicle control for the drug treatment experiments (M9 buffer for dopamine, distilled water for raclopride, DMSO for all other drugs).

C. elegans Lifespan

Synchronized larvae were plated onto NGM plates with food and grown for 2 days. Thirty L4 animals were picked and transferred to a fresh NGM plate containing floxuridine (FUDR) (0.005 mg/mL) with food (day 0). A total of 15 plates were prepared for each C. elegans strain (total N=450 animals per strain). Animals were scored as alive or dead every 2 days until no alive animals remained on the plate. An animal was marked as dead and removed from the plate if it no longer exhibited movement and did not respond to prodding with an eyelash pick. Censored animals included those C. elegans that could not be found (e.g., burrowed into the agar) or died due to desiccation on the walls of the plate. An individual animal's lifespan was the age at which it was scored as dead, and mean lifespan was calculated for each plate. Mean lifespan for each C. elegans strain was calculated as the average of the 15 plates.

### Enzyme-Linked Immunosorbent Assay (ELISA)

Synchronized larvae were plated onto NGM plates with food and grown for 4 days. Animals were collected, washed, and pelleted in M9 buffer. Animals were resuspended in ice-cold RIPA buffer (BioBasic, RB4478) containing a protease inhibitor cocktail (Roche, 11836153001) (1 mL of buffer per 0.1 g of C. elegans pellet) and then sonicated at 70% power for 2 runs of 10 seconds each (Qsonica, Q125). Sonicated samples were incubated at 4° C. for 2 hours in a shaker and then centrifuged at 17,000 g for 30 minutes. ELISAs were performed to measure α-synuclein protein levels in the protein lysate (supernatant). Total protein was quantified using a commercial BCA assay (ThermoFisher, 23227) according to the manufacturer's instructions. ELISAs were conducted using a commercial human α-synuclein ELISA kit (BioLegend, 844101) according to the manufacturer's instructions.

Dopaminergic Neuron Loss in C. elegans

Control (tzls3;egls1) and α-synuclein ([A30P α-synuclein];tzls3;egls1) gravid adult animals were synchronized, and eggs were hatched overnight (day 0) on NGM plates without food. Synchronized L1 larvae were transferred to NGM plates with food and grown at room temperature. L4 larval animals were then picked and transferred to fresh NGM plates with food for better synchronization. The animals were transferred to new plates every 48 to 72 hours. On day 3 to day 9, ADE neurons were scored from 26 to 58 animals in a blinded fashion by GFP fluorescence using a widefield microscope (Zeiss AxioObserver). Any absent or unidentifiable ADE neuron was scored as degenerated.

CRE-GFP Reporter of Dopaminergic Dysfunction in C. elegans

Animals were synchronized by isolating eggs using alkaline hypochlorite treatment and hatching overnight. Larvae were plated onto NGM agar plates with OP50 and grown for 3 to 4 days prior to CRE-GFP analysis. Animals were washed with M9 buffer until clear of bacteria and then resuspended in a small volume of M9 buffer. Animals were mounted on 10% agarose pads and immobilized on the pads with 0.2 to 0.5 μl of 0.1 μm diameter polystyrene microspheres (Polysciences, 00876-15). GFP fluorescence of SIA neurons was scored in a blinded fashion fluorescence using a widefield microscope (Zeiss AxioObserver). The number of animals with at least one GFP positive SIA neurons, as well as the number of GFP positive SIA neurons per animal.

Gene Ontology (GO) Analysis of C. elegans Coiler Strains

Yemini et al.¹¹ published a database of behavioural phenotypes for 305 C. elegans strains, including the coiler phenotype (reported as coiling frequency and coil time). Ninety-seven genes were associated with C. elegans strains having a statistically significant increase in either coiler frequency or coil time compared to N2 (binned q value of <0.05). These genes were analysed with PANTHER gene ontology “GO biological process complete” (pantherdb.org/geneListAnalysis.do). The C. elegans genome was used as the reference list. For all GO terms ≥100-fold enrichment, the biological processes were ranked by the −log10 of the false discovery rate (FDR).

### Immunoprecipitation and Immunoblotting

Synchronized animals (4 or 5 days old) were lysed in 0.5X Tris-HCl buffer with 0.5% Triton X-100 at 100° C. for 5 minutes and then passed through a 23-gauge needle 10 times. The lysate was centrifuged at 10,000 g for 10 minutes in a 4° C. refrigerated centrifuge, and the protein lysate (supernatant) was transferred to a new tube. Protein concentration was determined with a Bio-Rad Lowry protein assay (Bio-RAD, 5000116) according to the manufacturer's instructions. For immunoprecipitation, protein lysates were incubated on a rotator at 4° C. overnight with mouse anti-human α-synuclein (Clone 42, BD Laboratories, 610787; dilution 1:100). Immune complexes were isolated by the addition of washed protein G agarose beads followed by incubation for 4 hours at 4° C. Beads were washed and samples were analysed by immunoblotting and mass spectrometry (see below). For immunoblotting, protein lysates (9.5 μg) or beads from immunoprecipitations were mixed with 6X Laemmli sample buffer, and proteins were then separated on a 10% acrylamide gel by SDS-PAGE. Proteins were transferred to a PVDF membrane using a wet transfer system. The membranes were probed with anti-α-synuclein mouse monoclonal antibodies (Clone 42, BD Laboratories, 610787; dilution 1:500). Biotinylated goat anti-mouse antibodies (Jackson Immuno, 115065146; dilution 1:20,000) were used as secondary antibodies with streptavidin-conjugated with horseradish peroxidase (Jackson Immuno, 016030084; dilution 1:10,000). Signals were detected with ECL (ThermoFisher, 32132) and developed on HyBlot CL autoradiography film (DV-E3018).

Mass Spectrometry and GO Analysis of α-Synuclein-Interacting C. elegans Proteins

Liquid chromatography-tandem mass spectrometry (LC-MS/MS) was performed by SPARC BioCentre Molecular Analysis, The Hospital for Sick Children, Toronto, Canada using samples of proteins co-immunoprecipitated with α-synuclein were recorded (described above). One hundred and thirty-one proteins were identified in immunoprecipitants from α-synuclein C. elegans lysates but absent from GFP C. elegans lysates. The genes that encode these proteins were analysed with PANTHER gene ontology “GO biological ontology process complete” (pantherdb.org/geneListAnalysis.do). The C. elegans genome was used as the reference list. Biological processes with FDR ≤0.05 were ranked according to fold enrichment.

### In Silico Ranking of Drugs Predicted to Reduce α-Synuclein Oligomers

IBM Watson for Drug Discovery Predictive Analytics identified ˜26 million records in Medline that cited either the known or candidate entities. Every single Medline abstract was then converted into a multidimensional vector of the words and phrases contained in the document, relative to the 20,000 most common words and phrases within the English lexicon, using a term frequency-inverse document frequency statistic. A centroid for each known and candidate entity was then generated by averaging the multidimensional vectors of all documents associated with each entity and used to produce a distance matrix comprising a similarity index for every pair of entities. Finally, a graph diffusion algorithm was applied to rank each candidate entity by similarity to the entire known set rank, thus producing a ranked candidate list ordered by predicted semantic similarity to the known set. The model performance was validated using a leave-one-out (LOO) cross-validation in which the ranking process (as described above) was run 15 times, each time with one entity removed from the known set and added to the candidate set. Receiver-Operating Characteristic (ROC) and Precision-Recall curves were generated.

### Luciferase Protein-Fragment Complementation and Cell Viability Assays in Cell Lines

H4 neuroglioma cells (ATCC, HTB-148) were maintained in Dulbecco's Modified Eagle Medium plus high glucose, L-glutamine, and sodium pyruvate (ThermoFisher, 11995-065) with 10% heat-inactivated fetal bovine serum (ThermoFisher, 16140071) and 1% antibiotic-antimycotic cocktail (ThermoFisher, 15240062) at 37° C. and 5% CO₂. DNA expression constructs encoding for full-length human α-synuclein fused to the N-terminal fragment of Gaussia princeps luciferase (syn-luc1), full-length human α-synuclein fused to the C-terminal fragment of Gaussia princeps luciferase (syn-luc2), and full-length Gaussia princeps luciferase were kindly provided by Dr. Pamela McLean, Mayo Clinic Jacksonville.³³ Cells were transiently co-transfected with syn-luc1 and syn-luc2 or transfected with luciferase alone using Superfect transfection reagent (Qiagen, 301305) according to the manufacturer's instructions. Twenty-four hours after transfection, cells were plated on a 96-well microplate at 60,000 cells per well. Twenty-four hours after plating, cells were treated with drugs or vehicle control. Following 24 hours of treatment, cells were washed 1 time with PBS and bioluminescence was measured using an automated CLARIOstar plate reader (Mandel, 430-0505). Cell permeable, native coelenterazine was used as the Gaussia luciferase substrate. Lyophilized coelenterazine (Nanolight, 303-500) was reconstituted in NanoFuel Solvent (Nanolight, 399-1), then diluted in PBS to 16.6 μg/mL, and dispensed per well to a final concentration of 20 μM). The bioluminescent signal generated by the luciferase enzyme was assessed at 470 nm over 5 seconds. Raw luminescence units were normalized to the DMSO vehicle control of each 96-well microplate. To assess cell viability, H4 neuroglioma cells were prepared and treated as described above and, 1 hour prior to viability analysis, cells were treated with PrestoBlue viability reagent (ThermoFisher, A13261) according to the manufacturer's instructions. Fluorescence was automatically measured using a CLARIOstar plate reader (Mandel, 430-0505) and then normalized to the fluorescence measure of the DMSO vehicle control.

### Venus YFP Protein-Fragment Complementation Assay in Primary Cortical Neurons

Pregnant Sprague Dawley rats (E17) were purchased from Charles River. Embryos were surgically removed, and neurons were harvested in a sterile environment. Neurons were plated on poly-D-lysine coated glass coverslips at a density of 5×10⁵ cells per well in Neurobasal-A medium, (Gibco, 10888022) supplemented with B27 (2%) (Gibco, 17504044), antibiotic-antimycotic (1%) (Gibco, 15240062), and Glutamax (1%) (Gibco, 35050061). Fifty percent media changes were performed every 3 days. DNA expression constructs encoding for full-length human α-synuclein fused to the N-terminal fragment of Venus YFP (V1 S), full-length human α-synuclein fused to the C-terminal fragment of Venus YFP (SV2), and full-length Venus YFP were kindly provided by Dr. Pamela McLean, Mayo Clinic Jacksonville.³¹ The following AAV serotype 1/2 vectors were custom ordered from GeneDetect (Auckland, New Zealand): AAV1/2-CBA-V1 S-WPRE-BGH-polyA (AAV-V1 S), AAV1/2-CBA-SV2-WPRE-BGH-polyA (AAV-SV2), and AAV1/2-CBA-Venus YFP-WPRE-BGH-polyA (AAV-Venus). Two days post isolation, neurons were co-transduced with AAV-V1 S and AAV-SV2 or transduced with AAV-Venus alone at a MOI of 3,000. AAV-containing media was removed after 72 hours and cells were then treated with drugs or DMSO control for 72 hours at the following concentrations: acetaminophen (100 μM), caffeine (200 μM), losartan (100 μM), rapamycin (50 nM), rifabutin (20 μM). Concentrations were selected to avoid neuron toxicity according to previous reports.⁵⁷⁻⁶¹ Drug-containing media was removed, and cells were washed 1 time with ice-cold PBS before being fixed with 4% PFA (Sigma) at room temperature for 10 minutes. Levels of α-synuclein oligomers were measured by YFP fluorescence. To measure total α-synuclein levels, neurons were immunostained with anti-α-synuclein mouse monoclonal antibodies (Syn211, ThermoFischer, 32-8100; dilution 1:500) and goat anti-mouse secondary antibodies conjugated to Alexa Fluor® Plus 555 (ThermoFischer, A32727; dilution 1:500). Images of fixed cells were acquired using a confocal microscope equipped with 405, 488, 555, and 639 nm laser lines (Zeiss LSM880). All images for each biological replicate were taken within the linear range at constant gain and pinhole settings at a resolution of 1024×1024 pixels using a 63x/NA1.4 oil objective with the zoom set to 1.2. The imaging medium used was Zeiss Immersol 518F. YFP fluorescence intensity was analysed automatically using Imaris imaging software (Oxford Instruments).

### Stereotactic Surgery and Rifabutin Treatment of Rats

Adult female Sprague-Dawley rats (250-300 g) were purchased from Envigo. The animals were pair-housed in cages with wood bedding and access to food and water ad libitum. The animal colony was maintained in a regular 12-h light/dark cycle. All procedures were approved by the University Health Network Animal Care Committee in accordance with guidelines and regulations set by the Canadian Council on Animal Care. AAV serotype 1/2 was used to express A53 T α-synuclein (AAV-A53 T) under the control of the CAG promoter, a hybrid of the chicken beta actin (CBA) promoter fused with the cytomegalovirus (CMV) immediate early enhancer sequence (GeneDetect, Auckland, New Zealand), as previously described¹⁶³. An AAV1/2 vector lacking the A53 T α-synuclein open reading frame was used as an empty vector control (AAV-EV). Animals were secured in a stereotactic frame under isoflurane/oxygen anaesthesia (2.5% isoflurane and 1.5 L/min O₂) and anafen (5 mg/kg) analgesia. The surgical site was shaved and sterilized with iodine/betadine/isopropanol prior to making a 2-cm incision along the midline. The skull was exposed and a unilateral injection targeting the SN was performed at coordinates AP_5.2 mm, ML_2 mm, and DV_7.5 mm with respect to bregma. For each animal, a total volume of 1.5 μl of AAV-A53 T or AAVEV (5.1×10¹² genomic particles/ml) plus 0.5 μl of sterile PBS was injected at a rate of 0.5 μl/min using a microinjection pump and 10 μl Hamilton syringe with a 26-gauge needle. At the end of virus injection, the needle remained in place for 5 min before gradual removal.

Rats were randomly assigned to receive 25 mg/kg rifabutin (prepared as 1.5 mg/ml in 5% DMSO in saline) or vehicle control (5% DMSO in saline) daily. Treatments were started 2 days following stereotactic surgery. Rats were weighed and treated each day between 7:00-8:00 am with rifabutin or vehicle by intraperitoneal injection using a 25-gauge needle for 6 weeks.

### Collection of Rat Plasma, Cerebrospinal Fluid, and Brain Tissue

Animals were euthanised after 6 weeks of rifabutin or vehicle treatment by cardiac puncture under isoflurane/oxygen anaesthesia, followed by transcardial perfusion with approximately 100 to 200 ml of ice-cold heparinised saline. Blood obtained via cardiac puncture was centrifuged at 4° C. in microtainer tubes (BD, 365974) for 2 min at 10,000 g, and the upper layer plasma was collected, frozen on dry ice, and stored at −80° C. until use. Cerebrospinal fluid (CSF) was collected using a latex dropper bulb attached to a custom-made glass micropipette (Drummond Scientific, Broomall, PA) inserted into the cisterna magna. Approximately 50 to 100 μl of CSF were transferred into autoclaved vials and centrifuged at 3000 rpm over 3 to 5 s; samples with a detectable red blood cell precipitate were excluded due to blood contamination. CSF samples were frozen on dry ice and stored at −80° C. until use. Brains were then removed, and tissue anterior to the optic chiasm was snap frozen in dry ice-cooled isopentane. A single 1 mm thick section of the ventral striatum was immediately cut, using a matrix, and frozen on dry ice. These sections were sent to Vanderbilt University Neurochemistry Core (Nashville, TN, USA) for measurements of biogenic amines by high-performance liquid chromatography (HPLC). Approximately 100 mg of frozen brain tissue plus frozen plasma samples from a subset of animals were sent to InterVivo Solutions (Toronto, ON, Canada) for measurements of rifabutin concentrations by LC-MS/MS. Tissue posterior to the optic chiasm, including the posterior striatum and SN, was immersion-fixed in 4% paraformaldehyde in 0.1 M PBS for 2 days at room temperature and cryoprotected at 4° C. in 15% sucrose and then 30% sucrose in 0.1 M PBS solution until the brains sank. For immunostaining, 40 μm thick coronal cryosections were prepared using a sliding microtome (Leica Microsystems Inc.), and 6 series of sections were stored in cryoprotectant (30% glycerol, 30% ethylene glycol, 40% PBS) at −20° C. until use.

### Immunostaining of Brain Cryosections

Immunostaining for stereology was performed by washing free-floating sections with PBS-T (PBS with 0.1% Tween-20) three times for 10 min each at room temperature. Sections were then immersed in 3% H₂O₂ for 3 min to quench endogenous peroxidases. Sections were rinsed in PBS-T three times for 5 min each before incubation in blocking solution (2% BSA, 10% normal goat serum in PBS-T) for 1 h at room temperature. After blocking, sections were incubated with rabbit antityrosine hydroxylase (TH) antibodies (ThermoFisher Scientific, AB152; dilution 1:2000) in blocking solution overnight at room temperature. Sections were washed in TBS-T (TBS with 0.1% Tween-20) before incubation with alkaline phosphatase-conjugated goat anti-rabbit (H+L) secondary antibodies (Jackson ImmunoResearch, 111-055-144; dilution 1:500) in 2% NGS TBS-T for 2 h at room temperature. Sections were then washed three times for 5 min each in TBS-T before incubation in Vector Blue substrate, prepared by adding 2 drops of reagents 1, 2, and 3 to 5 ml of 100 mM Tris-HCl PH 8.2 (Alkaline Phosphatase Substrate Kit III, Vector Labs, SK-5003). The reaction was stopped by incubation of sections in 100 mM Tris-HCl PH 8.2 before the sections were washed five times for 3 min each in PBS. Sections were mounted onto slides and allowed to air-dry overnight. Slides were dehydrated by incubating for 3 min in ddH₂O, then for 1 min each in 70, 95, and 100% EtOH, and finally two times for 3 min each in Histoclear (Harleco, 65,351). Vectamount (Vector Labs, H-5000) was applied prior to coverslip application.

Immunofluorescent staining was performed by washing free-floating sections with PBS-T (0.2% Tween-20 or 0.1% Triton X-100) three times for 5 or 10 min each at room temperature. To detect total α-synuclein, sections were then incubated in blocking solution (2% BSA, 10% normal goat serum in PBS-T) for 1 h at room temperature. After blocking, sections were incubated with rabbit anti-TH antibodies (ThermoFisher Scientific, AB152; dilution 1:1000) and anti-α-synuclein mouse monoclonal antibodies specific for human α-synuclein (Syn211, ThermoFisher, 32-8100; dilution 1:500) in antibody solution (2% normal goat serum in PBS-T) overnight at room temperature. Sections were washed in PBS-T and incubated with secondary fluorescent antibodies in antibody solution for 1 h in the dark at room temperature. Secondary antibodies were Alexa Fluor goat anti-rabbit 594 (Invitrogen, A11037; dilution 1:500) and Alexa Fluor goat anti-mouse 488 (Invitrogen, A11029; dilution 1:500). To detect α-synuclein oligomers, sections were treated with 1 M glycine for 30 min and then incubated in blocking solution (1% BSA, 10% normal goat serum in PBS-T) for 1 h at room temperature. After blocking, sections were incubated with chicken anti-TH antibodies (Abcam, ab76442; dilution 1:1000) and Syn-02 antibodies (dilution 1:5000) in blocking solution overnight at room temperature. Syn-O2 is a mouse monoclonal antibody which specifically recognizes early soluble oligomers and late fibrils of α-synuclein, and Syn-02 has a high binding affinity for oligomeric α-synuclein164. Sections were washed in PBS and incubated with biotin-conjugated goat antimouse secondary antibodies (Jackson ImmunoResearch, 115-065-146; dilution 1:500) in PBS for 1 h at room temperature. Sections were washed in PBS and incubated with Alexa Fluor goat anti-chicken 594 (Invitrogen, A11042; dilution 1:500) and Alexa Fluor 488 streptavidin (Invitrogen, S32354; dilution 1:500) in PBS for 1 h in the dark at room temperature. Sections were washed in PBS, mounted onto glass slides, and allowed to dry. Fluorescence mounting medium (DAKO) was applied, followed by coverslip application. Appropriate targeting of AAV-A53 T injection in the SN was based on the findings from immunostaining human α-synuclein; animals were considered mistargeted and thus excluded from analyses if their SN cells were not transduced with AAV-A53 T.

### Image Analysis of Brain Cryosections

Imaging analyses were performed by a researcher blinded to the drug treatments. Confocal images of immunofluorescent staining were acquired with a Zeiss LSM700 confocal microscope equipped with 405, 488, 555, and 639 nm laser lines. All images were taken within the linear range at constant gain and the pinhole settings at optimal resolution settings determined by the software. The whole midbrain regions were imaged using a 10λ objective. Four serial coronal midbrain sections were imaged per animal, separated by 240 μm intervals. Confocal images of immunofluorescent stained midbrain sections were processed using HALO software (Indica Labs). Injected SN was selected as a region of interest (ROI), and dopaminergic neurons were identified by automated detection of TH-labelled cells within this ROI. Dopaminergic neuron densities, which correlate with neuronal counts obtained by conventional stereology as previously described 165, were estimated using HALO software. Levels of total human α-synuclein (detected by Syn211 staining) or α-synuclein oligomers (detected by Syn-O2 staining) were assessed in this ROI by measuring fluorescence intensity.

### Stereology of Brain Cryosections

The optical fractionator method was used for the unbiased stereological estimation of dopaminergic (i.e., TH-positive) cell counts in the injected and non-injectedSN (Stereo Investigator software version 9, MBF Biosciences). The investigator was blinded to the experimental groups. Every sixth section throughout the SN was quantified (7 sections total). The injected or noninjected SN was selected as a ROI bounded at a 10× objective, and counting was performed using a 40× oil immersion objective. A guard zone thickness of 2 μm was set at the top and bottom of each section. The sampling interval in the X-Y coordinate axis was set as follows: 175 μm×175 μm counting frame size; 300 μm×200 μm grid size; 20 μm dissector height. Coefficient of error was calculated according to Gundersen and Jensen¹⁶⁶, and values <0.10 were accepted.

### Real Time Quaking Induced Conversion (RT-QuIC) Assay

A modified version of the α-synuclein RT-QuIC assay described by Barger et al.¹⁶⁷ was used. Dilution of rat CSF (1:10) was performed with 1× N2 supplement (Thermo Fisher) diluted with PBS. Black 96-well, clear bottom plates were pre-loaded with six 1 mm in diameter silica beads per well (OPS Diagnostics). Lyophilized recombinant human α-synuclein (Sigma) was reconstituted to 1 mg/ml with filtered ddH₂O and centrifuged for 10 min at 4° C. through 100 kDa Amicon filters (Millipore). Diluted rat CSF (15 μl) was added to individual wells containing 85 μl of RT-QuIC reaction mixture composed of 40 mM NaPO4 pH 8.0 (Boston BioProducts), 170 mM NaCl, 0.1 mg/ml recombinant α-synuclein, 20 μM ThT (Sigma), and 0.0005% SDS. The plates were sealed with clear plastic film (Thermo Fisher) and incubated at 42° C. in Clariostar plate reader (BMG Labtech) with cycles of 1 min shaking (400 rpm, double orbital) and 1 min rest throughout the indicated incubation time. ThT fluorescence (450 nm excitation and 480 nm emission) was recorded every 45 min for 60 h. Replicate reactions were run in four separate wells for each sample. Positive RT-QuIC reactivity of individual wells was defined as enhanced ThT fluorescence above a predefined threshold at 60 h. This threshold was calculated as the mean background fluorescence of the diluent plus 5 standard deviations, equal to approximately 3200 relative fluorescence units (rfu) in our experiments. A sample was considered positive when at least one of the four replicates displayed positive ThT reactivity above the threshold. For each positive sample, the mean fluorescence intensity from the positive replicates was calculated and plotted against time. For each negative sample, the mean fluorescence intensity from the negative replicates was calculated and plotted against time.

### Example 7

In silico screening was carried out using Cyclica's Ligand Design™ Library Screening mode against a list of 572 drug compounds generated from drugs that are currently covered in the Ontario Drug Benefit formulary which may modulate Hsp70. The top 19 compounds from this screen were tested in vitro for their ability to reduce α-syn oligomerization using a luciferase protein-fragment complementation assay, and top hits were measured for cell viability using a presto-blue cytotoxicity assay. Drugs identified as non-cytotoxic were analyzed for ability to reduce α-syn oligomerization, effect on HSP70 immunofluorescence in primary cortical rat neurons using a venus YFP protein-fragment complementation assay, and effect on pathological α-syn-induced coiling in C. elegans worms.

Library screening identified a ranked list of 19 compounds predicted to interact with molecular chaperone HSP70. Of these 19 drugs, 7 hits were identified for reduction of α-syn oligomerization in H4 neuroglioma cells through reduction of luciferase fluorescence in protein-fragment complementation assays. Of these 7 hits, 3 drugs (Abacavir, Lamivudine and Acebutolol; dose range 0-20 μM) demonstrated no cytotoxicity. Abacavir (10 μM) was further shown to significantly reduce α-syn oligomerization and increase HSP70 levels in primary cortical rat neurons. Lastly Abacavir was tested in a C. elegans model of PD and found to also significantly reduce α-syn-induced coiling and early motor deficit.

### Example 8

### Predicting Repurposing Candidates for HSPA1A

MatchMaker, a deep-learning methodology, was used to generate the polypharmacological profiles of 572 small molecules. The polypharmacological profile consists of a rank-ordered list of proteins predicted according to their likelihood of binding Cyclica's structurally characterized proteome (8,642 proteins). The resulting database therefore consists of the binding profiles of 572 small molecules against the 8,642 proteins. From the 572 small molecules assessed, the top 25 compounds which yielded the highest rank percentiles (ranging from 99.61% to 97.94%) for HSPA1A were considered for follow-up experimental validation (Table 6).

Methods are described in Example 12.

### Example 9

### Abacavir, Acebutolol, and Lamivudine Reduce α-Syn Oligomers In Vitro

To begin to validate the in silico predictions, we selected the top 19 ranked drugs predicted to interact with HSP70 from the 572 screened to test in vitro (Table 6). We used a bioluminescent protein-fragment complementation assay in which human H4 neuroglioma cells co-express human α-synuclein tagged with the N- or C-terminal half of Gaussia princeps luciferase (FIG. 12). Reconstitution of a complete and active luciferase molecule from the fragments occurs upon α-synuclein oligomerization, and so luminescence provides an indirect measure of α-synuclein oligomer levels. H4 neuroglioma cells were co-transfected with α-syn luciferase fragments, and constructs were plated into a 96-well plate and treated with either 1 μM or 10 μM of each of the 19 drug compounds. Consistent with previous findings (Examples 1-6), 2 μM rapamycin treatment caused a decrease in α-syn oligomerization, whereas treatment with 50 nM of autophagy inhibitor bafilomycin A1 resulted in an increase in α-syn oligomers (FIG. 12A-B). A significant decrease in α-syn oligomers as measured by luciferase activity was found for the following 7 compounds: Abacavir, Acebutolol, Benzydamine, Hydralazine, Clioquinol, Lamivudine, and Modafinil (FIG. 12A-B).

TABLE 6
Top 25 compounds from the Ontario Drug Benefit (ODB) program which yielded the highest rank percentiles for HSPA1A.
Entry of drug simplified molecular-input line-entry system (SMILES) ID into Cyclica's deep learning ligand screening
against HSP70 (human HSP71A) input generated a ranked list of drugs most likely to interact with HSP70.

| | | Rank |
|---|---|---|
| Name | SMILES | Percentile |
| Trimebutine | CCC(COC(═O)C1═CC(═C(C(═C1)OC)OC)OC)(C2═CC═CC═C2)N(C)C | 99.61 |
| Eslicarbazepine | C1[C@@H](C2═CC═CC═C2N(C3═CC═CC═C31)C(═O)N)O | 99.57 |
| Epinephrine | CNC[C@@H](C1═CC(═C(C═C1)O)O)O | 99.42 |
| Pantoprazole | COC1═C(C(═NC═C1)CS(═O)C2═NC3═C(N2)C═C(C═C3)OC(F)F)OC | 99.31 |
| Lorazepam | C1═CC═C(C(═C1)C2═NC(C(═O)NC3═C2C═C(C═C3)Cl)O)Cl | 99.31 |
| Clobazam | CN1C(═O)CC(═O)N(C2═C1C═CC(═C2)Cl)C3═CC═CC═C3 | 99.26 |
| Tetracycline | C[C@@]1([C@H]2C[C@H]3[C@@H](C(═O)C(═C([C@]3(C(═ | 99.25 |
| | O)C2═C(C4═C1C═CC═C4O)O)O)O)C(═O)N)N(C)C)O | |
| Lamivudine | C1[C@H](O[C@H](S1)CO)N2C═CC(═NC2═O)N | 98.96 |
| Iodochlorhydroxyquin | C1═CC2═C(C(═C(C═C2Cl)I)O)N═C1 | 98.70 |
| Hydralazine | C1═CC═C2C(═C1)C═NN═C2NN | 98.67 |
| Celecoxib | CC1═CC═C(C═C1)C2═CC(═NN2C3═CC═C(C═C3)S(═O)(═O)N)C(F)(F)F | 98.66 |
| Clotrimazole | C1═CC═C(C═C1)C(C2═CC═CC═C2)(C3═CC═CC═C3Cl)N4C═CN═C4 | 98.51 |
| Zolpidem | CC1═CC═C(C═C1)C2═C(N3C═C(C═CC3═N2)C)CC(═O)N(C)C | 98.48 |
| Zopiclone | CN1CCN(CC1)C(═O)OC2C3═NC═CN═C3C(═O)N2C4═NC═C(C═C4)Cl | 98.43 |
| Abacavir | C1CC1NC2═C3C(═NC(═N2)N)N(C═N3)[C@@H]4C[C@@H](C═C4)CO | 98.41 |
| Lopinavir | CC1═C(C(═CC═C1)C)OCC(═O)N[C@@H](CC2═CC═CC═C2)[C@H](C[C@H](CC3═ | 98.38 |
| | CC═CC═C3)NC(═O)[C@H](C(C)C)N4CCCNC4═O)O | |
| Galantamine | CN1CC[C@@]23C═C[C@@H](C[C@@H]2OC4═C(C═CC(═C34)C1)OC)O | 98.31 |
| Benzydamine | CN(C)CCCOC1═NN(C2═CC═CC═C21)CC3═CC═CC═C3 | 98.29 |
| Thiamine | CC1═C(SC═[N+]1CC2═CN═C(N═C2N)C)CCO | 98.22 |
| Modafinil | C1═CC═C(C═C1)C(C2═CC═CC═C2)S(═O)CC(═O)N | 98.09 |
| Chloroquine | CCN(CC)CCCC(C)NC1═C2C═CC(═CC2═NC═C1)Cl | 98.08 |
| Oxazepam | C1═CC═C(C═C1)C2═NC(C(═O)NC3═C2C═C(C═C3)Cl)O | 98.03 |
| Acebutolol | CCCC(═O)NC1═CC(═C(C═C1)OCC(CNC(C)C)O)C(═O)C | 98.03 |
| Clorazepate | C1═CC═C(C═C1)C2═NC(C(═O)NC3═C2C═C(C═C3)Cl)C(═O)O | 98.00 |
| Tolterodine | CC1═CC(═C(C═C1)O)[C@H](CCN(C(C)C)C(C)C)C2═CC═CC═C2 | 97.94 |

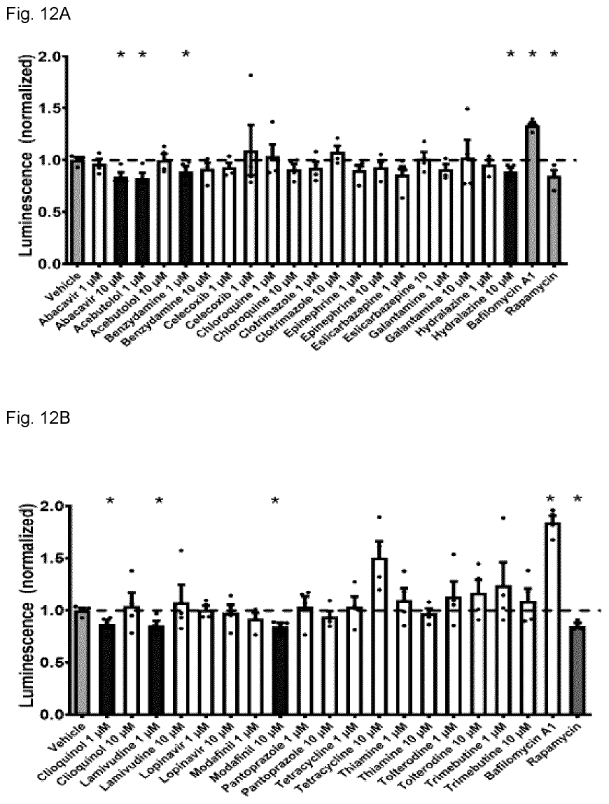

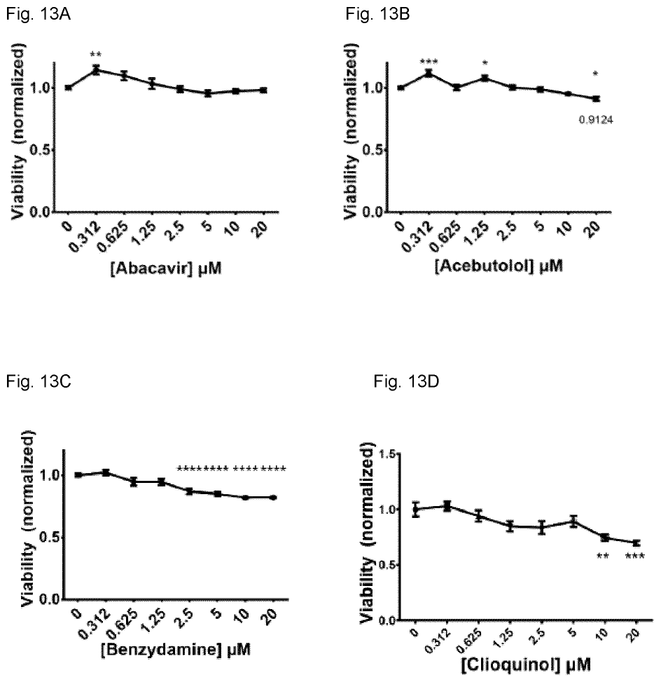

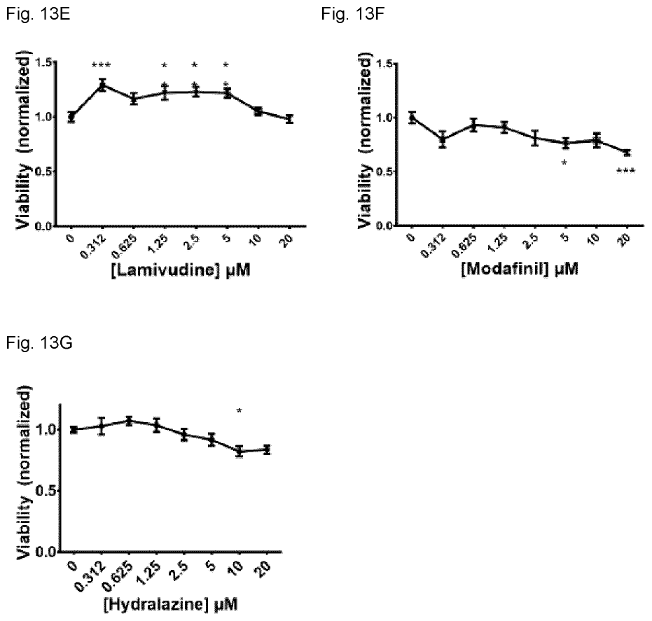

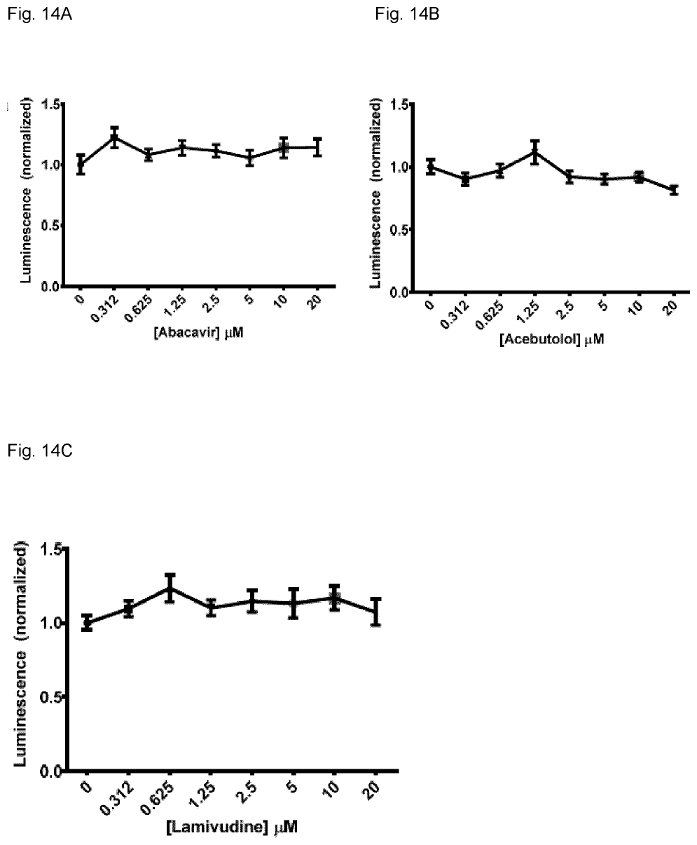

Since a reduction in luciferase activity in this assay can result due to cell death resulting in lower numbers of surviving α-syn-expressing cells, it was examined whether the above compounds caused cytotoxicity at the concentrations associated with reduced luciferase readings. Cells were treated with the seven positive hits, and cell viability was measured using a cell permeable resazurin-based reagent. Abacavir, Acebutolol, and Lamivudine exhibited no statistically significant reductions in cell viability (FIG. 13A, B, E). In contrast, treatment with Benzydamine (18% decrease), Clioquinol (30% decrease), Hydralazine (18% decrease), and Modafinil (32% decrease) resulted in reduced cell viability (FIG. 13C, D, F, G). To exclude the possibility that these drugs were directly affecting luciferase enzyme activity instead of reducing protein-fragment complementation assay, the effects of Abacavir, Acebutolol, and Lamivudine on H4 cells transfected with wild-type Gaussia luciferase alone were measured. None of these compounds reduced the activity of full-length luciferase on treated cells expressing luciferase alone (FIG. 14C). Thus, it can be concluded that abacavir, acebutolol and lamivudine can each individually reduce α-synuclein oligomers independent of cell viability reduction in vitro.

Methods are described in Example 12.

### Example 10

### Abacavir Reduces α-Syn Oligomers and Elevates HSP70 Levels in Neurons

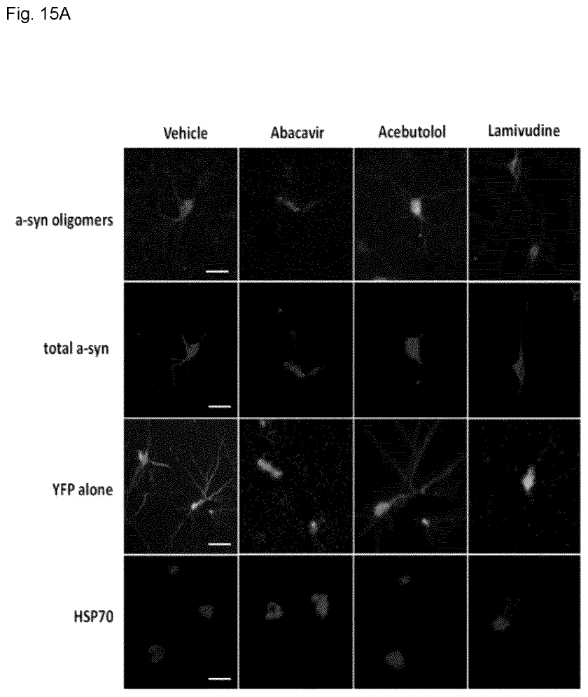

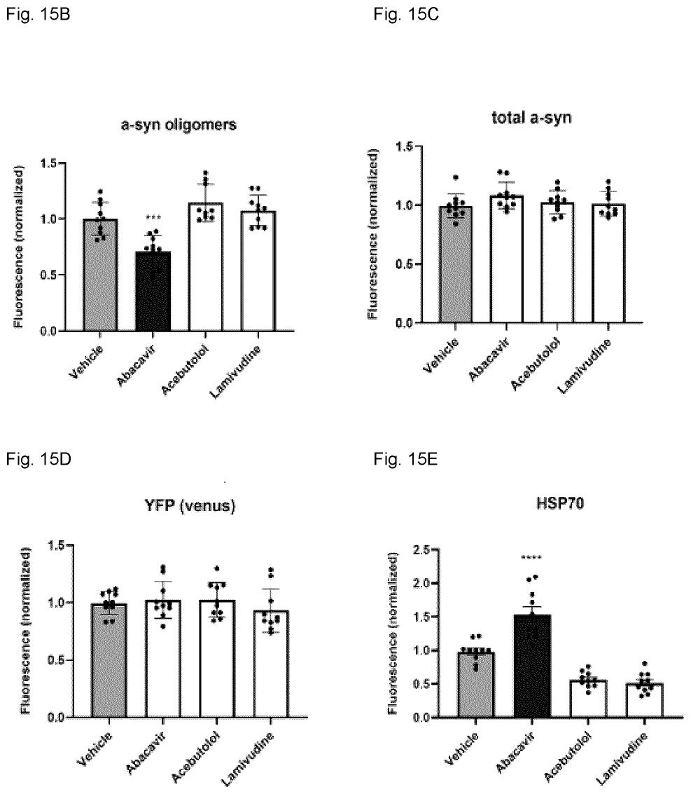

The effects of Abacavir, Acebutolol, and Lamivudine on α-syn oligomers were investigated in a neuronal system. Adeno-associated viral vectors (AAVs) were used to co-express human α-syn tagged with the N- or C-terminal half of Venus YFP in primary rat cortical neurons. Similar to the luciferase-based protein-fragment complementation system used in H4 cells described above, reconstitution of a complete YFP molecule from the YFP halves occurs upon α-syn oligomerization and thus spontaneous fluorescence provides an estimate of α-syn oligomer levels (Danzer et al., 2011; Dimant et al., 2013; Outeiro et al., 2008; Dimant et al., 2013. In neurons treated with Abacavir, YFP fluorescence was significantly reduced compared with vehicle control treated cells. No significant differences were observed in YFP fluorescence following Acebutolol or Lamivudine treatment (10 μm) compared to vehicle control (FIG. 15A-B) No change in total α-syn levels was observed with any of the drug treatments (FIG. 15C). Furthermore, none of the compounds reduced YFP fluorescence when treated neurons expressed full-length YFP alone (FIG. 15D).

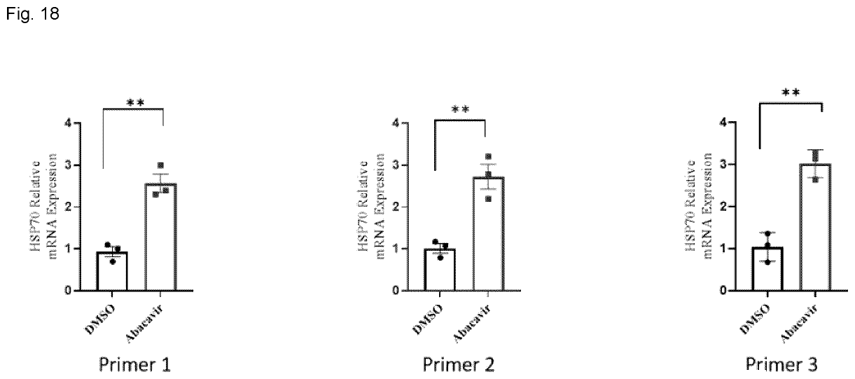

To investigate the mechanism underlying these drug effects, experiments were directed towards the potential involvement of molecular chaperone HSP70. HSP70 is known to play an essential role in suppressing α-synuclein aggregation, and in particular the inhibition of α-synuclein oligomer formation (Tao et al., 2020). To investigate the in silico predictions of HSP70 interactions with top drug candidates from the Cyclica proteome screening, fluorescence intensity was measured in V1 S-SV2 primary cortical rat neurons (FIGS. 15A and E) for potential effects on α-synuclein oligomers. Abacavir treatment (10 μM) significantly increased HSP70 fluorescence compared to vehicle control (FIGS. 15A and E). By performing real-time quantitative RT-PCR, it was found that neurons treated with Abacavir had increased HSP70 mRNA levels compared with neurons treated with vehicle control (FIG. 18). Since previous studies have demonstrated that an elevation of HSP70 reduces α-synuclein aggregation, we therefore suggest that Abacavir could mediate it's α-synuclein reducing effects by increasing levels of the chaperone protein through enhanced HSP70 transcription.

Methods are described in Example 12.

### Example 11

Abacavir Reduces α-Syn Dependent Coiler Phenotype in C. elegans

Previously, mild coiling has been demonstrated as a motor phenotype in C. elegans expressing mutant α-syn. Specifically, this early motor phenotype in transgenic α-synuclein C. elegans, as measured using an unbiased and semi-automated quantification methods, has been demonstrated to result from dopaminergic neuron dysfunction that precedes neuronal loss. In addition, this motor phenotype has been shown to be responsive to drugs which inhibit α-synuclein oligomers (Examples 1-6).

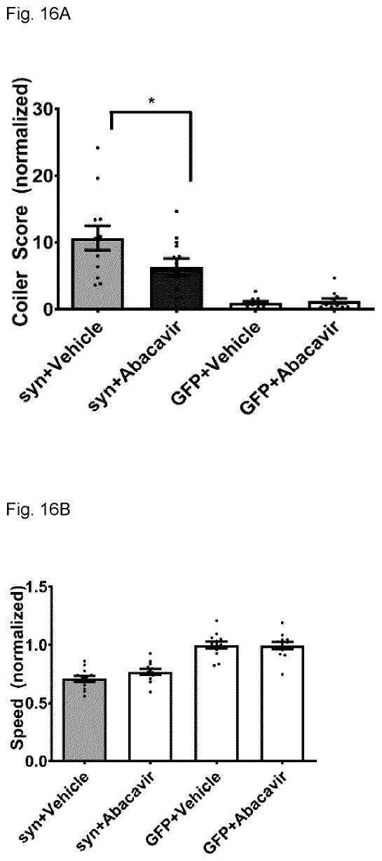

To investigate whether abacavir elicits an α-syn dependent motor phenotype, the coiler score of C. elegans was measured following 72 hr drug treatment. Abacavir (100 μm) significantly reduced coiler score in α-syn worms compared to DMSO vehicle control (FIG. 16A). No significant differences were found in C. elegans speed in abacavir treated worms compared to DMSO control (FIG. 16B). Additionally, no differences were found in abacavir treated GFP worms compared to α-syn worms for coiler score or speed (FIG. 16A-B). Taken together this suggests that abacavir can rescue an early motor phenotype caused by mutant α-syn toxicity.

Methods are described in Example 12.

This study sought to investigate in silico predictions of α-syn oligomer reducing compounds for potential repurposing in neurodegenerative diseases such as PD.

Prior art studies show correlation of PD disease prevalence does not necessarily indicate a targeted therapeutic benefit for repurposing these drugs. The application of artificial intelligence that led to the discovery of antiviral abacavir as a hit for potential repurposing in PD is one that would not have been identified previously using conventional hit-to-target pre-clinical research, using targeted small-molecule ligand interactions.

The potential effects of abacavir on α-synuclein in PD has yet to be investigated in the literature, and this study provides an unexpected result in the ability of abacavir to reduce α-syn oligomerization in primary cortical rat neurons.

The AI methods employed in this study predicted the binding of the 572 ODB drug list against the structurally characterised human proteome, to generate the polypharmacological profile for molecular chaperone HSP70. It's estimated that drugs can interact with around 30-300 proteins in vivo, in addition to their pharmacological target (Zhou et al., 2015). Despite this, many traditional in silico drug discovery techniques, such as structure-based (Ehrt et al., 2016), network based (Jin et al., 2012) or molecular docking-based approaches (Gentile et al., 2020) focus on a single drug target, leaving polypharmacology largely unexplored. This in silico technique can be translated for not only neurodegenerative associated proteins, but across a number of disease types.

### Example 12

### Methods

### In Silico Ligand-Protein Interaction Screening Against HSP70

From the Ontario Drug Benefit (ODB) program library, non-small molecule entries were removed, yielding a total of 572 molecules which were screened for predictive HSPA1A activity using Cyclica's MatchMaker predictive engine (Michael et al., 2021; Cyclica, 2021; Redka et al., 2020). MatchMaker is a deep-learning model for predicting drug-target binding interactions on the basis of the drug and target protein pocket's structural features. The model is trained on millions of known interactions, mapped onto likely binding sites obtained from 100,000 s of experimentally-determined protein structures and homology models. The collection of 572 small molecule compounds was cross-screened with a pre-constructed library of 8642 structurally-characterized proteins, evaluating 4,960,384 total pairwise drug-target interactions. Screening was performed with the 2020Q1 MatchMaker release model and human proteome. Small molecules ranking the target protein HSPA1A favourably relative to the remainder of the cross-screened proteome (polypharmacological profile) were selected for further experimental analysis.

### In Vitro Assay of α-Syn Oligomers in Rat Neurons

Pregnant Sprague Dawley rats (E17) were purchased from Charles River. Embryos were surgically removed from the euthanized female and neurons were harvested in a sterile environment. Cells were plated onto poly-D-lysine coated glass coverslips at a density of 0.5 million cells/well with half media changes every 3 days. Viral transduction of the primary neurons was conducted using adeno-associated virus (AAV) serotype 1/2 (GeneDetect) encoding for α-syn tagged to fragments of Venus YFP as previously described (Dimant et al., 2013). Neurons were co-transduced with AAV-V1 S (expressing amino acids 1 to 158 of Venus YFP fused to the N-terminus of full-length α-syn) and AAV-SV2 (expressing amino acids 159 to 239 of Venus YFP to the C-terminus of full-length α-syn) or transduced with AAV-Venus alone 2 days post isolation at an infection multiplicity of 3000. AAV-containing media was removed after 72 hours and cells were treated with drugs (10 μM) or vehicle alone (5% DMSO) for 72 hours. Drug concentration was selected to avoid neuronal toxicity (Giunta et al., 2011; Li et al. 2018; Nooka et al,. 2017). After 72 hours of treatment, drug-containing media was removed and cells were washed once with ice-cold PBS before being fixed with 4% paraformaldehyde (Sigma) for 10 minutes at room temperature. Following fixation, cells were washed 3 times with ice cold PBS and permeabilized using 0.1% Triton X-100 for 10 minutes at room temperature. After permeabilization, cells were washed 3 times with PBS and incubated in blocking solution (1% BSA, 22.52 mg/ml glycine, 0.1% Tween-20, all dissolved in PBS) for 1 hour at room temperature. Cells were then incubated with monoclonal mouse α-synuclein primary antibody (Clone Syn 211, ThermoFisher, #32-8100, dilution 1:500) and rabbit HSP70 primary antibody (Enzo, #ADI-SPA-812-F, dilution 1:500) in blocking solution overnight at 4° C. before being washed 3 times with PBS. Secondary antibody Alexa Fluor 555 Goat Anti-Mouse (Thermo Fisher, #A28180, dilution 1:500) or Alexa Fluor 647 Goat Anti-Rabbit (ThermoFisher, #A21247, dilution 1:500) in blocking solution and added to cells for 1 hour at room temperature. Cells were washed three more times before being counter-stained with DAPI for 5 mins and mounted using ProLong Gold mounting medium for fluorescence (ThermoFisher). Images of fixed cells were acquired using a Zeiss LSM880 confocal microscope equipped with 405, 488, 555 and 639 nm laser lines. All images for each biological replicate were taken within the linear range at constant gain and pinhole settings using a 63x/NA1.4 oil objective. The imaging medium used was Zeiss Immersol 518F. Fluorescence intensity was analysed automatically using Imaris imaging software (Oxford Instruments) by measuring the mean fluorescence intensity of each individual fluorescent neuron using the “surfaces” module. This module detects fluorescence above background threshold in each field of view, identifies a cell body and neuronal projections of each cell, and calculates the mean fluorescence intensity of each fluorescent cell within the field of view. The mean fluorescence intensity of 10 fields of view was recorded for each biological replicate and normalized to the mean intensity of the DMSO treated condition. The value generated for each neuron was normalized to the mean of fluorescence intensity of neurons treated with vehicle alone. All scale bars represent 10 μm.

### Real-Time Quantitative RT-PCR

Total RNA was isolated from the cultured neurons on DIV 5 using RNeasy Mini Kit (50) (Qiagen, Cat #74104) according to the manufacturer's instructions. RNA quantity and purity were determined by spectrophotometric analysis (NanoDrop 2000c; Thermo Scientific). One microgram of purified RNA was reversely transcribed to cDNA using random primer (Invitrogen) and Superscript™ III Reverse Transcriptase (Invitrogen Cat #18080044). Quantitative real-time PCR was performed in the Quant Studio™ 5 Real-Time PCR System (ThermoFisher Scientific) using Power Track™ SYBR Green Master Mix (Invitrogen, Cat #A46109).

Statistical analysis: For the real-time quantitative RT-PCR, the following equations were used: ΔCt=Ct (gene of interest)−Ct (house keeping gene), ΔΔCt=ΔCt (sample)−ΔCt (calibrator), relative quantity (RQ)=2−ΔΔCt. RQ value was set at 1 for the DMSO treated group when compared to Abacavir treated group. Data were obtained from three biological repeats (n=3) and presented as mean±SEM. The statistical significance is obtained by unpaired student's t-test (two-tailed) where ** indicates p value <0.01.

### In Vivo Assay of α-Syn-Mediated Dopaminergic Dysfunction Using C. Elegans

C. elegans were grown and maintained as previously described (Examples 1-6). Synchronized L1 larvae were grown for 2 days under standard growth conditions then treated on plates spread with OP50 culture containing either abacavir (100 μM) or vehicle (0.5% DMSO). After 72 hours of drug treatment, animals were randomly picked, washed, and recorded. A total of ten worms were recorded per population, and twelve populations were recorded for each of the four treatment groups. Locomotion was recorded for each population over a total of 1800 frames over three minutes. The animal's movement speed, area, and perimeter were automatically analyzed as previously described. Coiler score was calculated automatically using a ratio of the animal's area to perimeter, where a higher coiler score would represent a greater degree of α-synuclein-induced dopaminergic neuron dysfunction in vivo.

### Statistical Analysis

All statistical analyses were conducted using GraphPad Prism 6 or 8. Mean±SEM with individual replicates are presented when appropriate. The specific statistical tests were performed are indicated.

### Example 13

### Example of the Use of Abacavir, Lamivudine, and Losartan in Rats

Abacavir, lamivudine, and losartan are tested in the in vivo rat model using similar methods to those described in Example 6, wherein the rats are administered abacavir, losartan or lamivudine instead of rifabutin.

### Example 14

### Example of the Use of Rifabutin, Abacavir, Lamivudine, or Losartan in Human Patients

One or more dosages of rifabutin, abacavir, lamiduvine, and/or losartan is/are administered to a group of human subjects, for example in a clinical trial. For example, the dosage(s) may be according to their current administration protocol(s) for their currently approved indication(s). Other dosages may also be tested.

### REFERENCES

1. Dorsey, E. R. & Bloem, B. R. The Parkinson pandemic—a call to action. JAMA Neurol. 75, 9-10 (2018).
2. Kalia, L. V. & Lang, A. E. Parkinson's disease. Lancet 386, 896-912 (2015).
3. Brundin, P., Dave, K. D. & Kordower, J. H. Therapeutic approaches to target alpha-synuclein pathology. Exp. Neurol. 298, 225-235 (2017).
4. Lang, A. E. & Espay, A. J. Disease modification in Parkinson's disease: current approaches, challenges, and future considerations. Mov. Disord. 33, 660-677 (2018).
5. Kordower, J. H. & Burke, R. E. Disease modification for Parkinson's disease: axonal regeneration and trophic factors. Mov. Disord. 33, 678-683 (2018).
6. O'Reilly, L. P., Luke, C. J., Perlmutter, D. H., Silverman, G. A. & Pak, S. C. C. elegans in high-throughput drug discovery. Adv. Drug Deliv. Rev. 69-70, 247-253 (2014).
7. Visanji, N. P. et al. α-Synuclein-based animal models of Parkinson's disease: challenges and opportunities in a new era. Trends Neurosci. 39, 750-762 (2016).
8. van Ham, T. J. et al. C. elegans model identifies genetic modifiers of alpha-synuclein inclusion formation during aging. PLoS Genet. 4, e1000027 (2008).
9. Kuwahara, T. et al. Familial Parkinson mutant alpha-synuclein causes dopamine neuron dysfunction in transgenic Caenorhabditis elegans. J. Biol. Chem. 281, 334-340 (2006).
10. Tucci, M. L., Harrington, A. J., Caldwell, G. A. & Caldwell, K. A. Modeling dopamine neuron degeneration in Caenorhabditis elegans. Methods Mol. Biol. 793, 129-148 (2011).
11. Yemini, E., Jucikas, T., Grundy, L. J., Brown, A. E. X. & Schafer, W. R. A database of Caenorhabditis elegans behavioral phenotypes. Nat. Methods 10, 877-879 (2013).
12. Stephens, G. J., Johnson-Kerner, B., Bialek, W. & Ryu, W. S. Dimensionality and dynamics in the behavior of C. elegans. PLoS Comput. Biol. 4, e1000028 (2008).
13. Schneider, C. A., Rasband, W. S. & Eliceiri, K. W. NIH Image to ImageJ: 25 years of image analysis. Nat. Methods 9, 671-675 (2012).
14. Koushika, S. P. et al. A post-docking role for active zone protein Rim. Nat. Neurosci. 4, 997-1005 (2001).
15. Su, C.-W. et al. The short coiled-coil domain-containing protein UNC-69 cooperates with UNC-76 to regulate axonal outgrowth and normal presynaptic organization in Caenorhabditis elegans. J. Biol. 5, 9 (2006).
16. Alfonso, A., Grundahl, K., Duerr, J. S., Han, H. P. & Rand, J. B. The Caenorhabditis elegans unc-17 gene: a putative vesicular acetylcholine transporter. Science 261, 617-619 (1993).
17. Huang, C., Xiong, C. & Kornfeld, K. Measurements of age-related changes of physiological processes that predict lifespan of Caenorhabditis elegans. Proc. Natl. Acad. Sci. U.S.A 101, 8084-8089 (2004).
18. Cooper, J. F. et al. Delaying aging is neuroprotective in Parkinson's disease: a genetic analysis in C. elegans models. NPJ Park. Dis. 1, 15022 (2015).
19. Bodhicharla, R. et al. Effects of α-synuclein overexpression in transgenic Caenorhabditis elegans strains. CNS Neurol. Disord. Drug Targets 11, 965-975 (2012).
20. Vartiainen, S., Aarnio, V., Lakso, M. & Wong, G. Increased lifespan in transgenic Caenorhabditis elegans overexpressing human alpha-synuclein. Exp. Gerontol. 41, 871-876 (2006).
21. Rosborough, K., Patel, N. & Kalia, L. V. α-Synuclein and parkinsonism: updates and future perspectives. Curr. Neurol. Neurosci. Rep. 17, 31 (2017).
22. Blesa, J., Phani, S., Jackson-Lewis, V. & Przedborski, S. Classic and new animal models of Parkinson's disease. J. Biomed. Biotechnol. 2012, 845618 (2012).
23. Langston, J. W., Langston, E. B. & Irwin, I. MPTP-induced parkinsonism in human and non-human primates-clinical and experimental aspects. Acta Neurol. Scand. Suppl. 100, 49-54 (1984).
24. Braungart, E., Gerlach, M., Riederer, P., Baumeister, R. & Hoener, M. C. Caenorhabditis elegans MPP+model of Parkinson's disease for high-throughput drug screenings. Neurodegener. Dis. 1, 175-183 (2004).
25. Suo, S., Kimura, Y. & Van Tol, H. H. M. Starvation induces cAMP response element-binding protein-dependent gene expression through octopamine-Gq signaling in Caenorhabditis elegans. J. Neurosci. 26, 10082-10090 (2006).
26. Suo, S., Culotti, J. G. & Van Tol, H. H. M. Dopamine counteracts octopamine signalling in a neural circuit mediating food response in C. elegans. EMBO J. 28, 2437-2448 (2009).
27. Kimura, Y. et al. A CaMK cascade activates CRE-mediated transcription in neurons of Caenorhabditis elegans. EMBO Rep. 3, 962-966 (2002).
28. Manecka, D.-L., Vanderperre, B., Fon, E. A. & Durcan, T. M. The neuroprotective role of protein quality control in halting the development of alpha-synuclein pathology. Front. Mol. Neurosci. 10, (2017).
29. Putcha, P. et al. Brain-permeable small-molecule inhibitors of Hsp90 prevent alpha-synuclein oligomer formation and rescue alpha-synuclein-induced toxicity. J. Pharmacol. Exp. Ther. 332, 849-857 (2010).
30. Moussaud, S. et al. Targeting alpha-synuclein oligomers by protein-fragment complementation for drug discovery in synucleinopathies. Expert Opin. Ther. Targets 19, 589-603 (2015).
31. Danzer, K. M. et al. Heat-shock protein 70 modulates toxic extracellular α-synuclein oligomers and rescues trans-synaptic toxicity. FASEB J. 25, 326-336 (2011).
32. Shulman, J. M., Shulman, L. M., Weiner, W. J. & Feany, M. B. From fruit fly to bedside: translating lessons from Drosophila models of neurodegenerative disease. Curr. Opin. Neurol. 16, 443-449 (2003).
33. Kalia, L. V. et al. Ubiquitinylation of α-synuclein by carboxyl terminus Hsp70-interacting protein (CHIP) Is regulated by Bcl-2-associated athanogene 5 (BAG5). PLoS ONE 6, e14695 (2011).
34. Pushpakom, S. et al. Drug repurposing: progress, challenges and recommendations. Nat. Rev. Drug Discov. 18, 41-58 (2018).
35. Chen, Y., Elenee Argentinis, J. & Weber, G. I B M Watson: how cognitive computing can be applied to big data challenges in life sciences research. Clin. Ther. 38, 688-701 (2016).
36. Bakkar, N. et al. Artificial intelligence in neurodegenerative disease research: use of IBM Watson to identify additional RNA-binding proteins altered in amyotrophic lateral sclerosis. Acta Neuropathol. 135, 227-247 (2018).
37. Jarvela, T. S. et al. The neural chaperone proSAAS blocks α-synuclein fibrillation and neurotoxicity. Proc. Natl. Acad. Sci. 113, E4708-E4715 (2016).
38. Aelvoet, S.-A. et al. Noninvasive bioluminescence imaging of α-synuclein oligomerization in mouse brain using split firefly luciferase reporters. J. Neurosci. 34, 16518-16532 (2014).
39. Eschbach, J. et al. Mutual exacerbation of PGC-1a deregulation and α-synuclein oligomerization. Ann. Neurol. 77, 15-32 (2015).
40. Lu, J.-H. et al. Baicalein inhibits formation of α-synuclein oligomers within living cells and prevents A B peptide fibrillation and oligomerisation. Chembiochem. 12, 615-624 (2011).
41. Takahashi, R. et al. Phenolic compounds prevent the oligomerization of α-synuclein and reduce synaptic toxicity. J. Neurochem. 134, 943-955 (2015).
42. Wagner, J. et al. Anle138b: a novel oligomer modulator for disease-modifying therapy of neurodegenerative diseases such as prion and Parkinson's disease. Acta Neuropathol. 125, 795-813 (2013).
43. Lu, J.-H. et al. Isorhynchophylline, a natural alkaloid, promotes the degradation of alpha-synuclein in neuronal cells via inducing autophagy. Autophagy 8, 98-108 (2012).
44. Outeiro, T. F. et al. Formation of toxic oligomeric α-synuclein species in living cells. PLoS ONE 3, e1867 (2008).
45. Decressac, M. et al. TFEB-mediated autophagy rescues midbrain dopamine neurons from α-synuclein toxicity. Proc. Natl. Acad. Sci. U.S.A 110, E1817-E1826 (2013).
46. Klucken, J. et al. Alpha-synuclein aggregation involves a bafilomycin A1-sensitive autophagy pathway. Autophagy 8, 754-766 (2012).
47. Saha, S. et al. Mutations in LRRK2 potentiate age-related impairment of autophagic flux. Mol. Neurodegener. 10, 26 (2015).
48. Dimant, H. et al. Direct detection of alpha synuclein oligomers in vivo. Acta Neuropathol. Commun. 1, 6 (2013).
49. Koprich, J. B., Kalia, L. V. & Brotchie, J. M. Animal models of α-synucleinopathy for Parkinson disease drug development. Nat. Rev. Neurosci. 18, 515-529 (2017).
50. Ramot, D., Johnson, B. E., Berry, T. L., Carnell, L. & Goodman, M. B. The Parallel Worm Tracker: a platform for measuring average speed and drug-induced paralysis in nematodes. PloS One 3, e2208 (2008).
51. Swierczek, N. A., Giles, A. C., Rankin, C. H. & Kerr, R. A. High-throughput behavioral analysis in C. elegans. Nat. Methods 8, 592-598 (2011).
52. Tyson, T. et al. Novel animal model defines genetic contributions for neuron-to-neuron transfer of α-synuclein. Sci. Rep. 7, 7506 (2017).
53. Bae, E.-J. et al. LRRK2 kinase regulates α-synuclein propagation via RAB35 phosphorylation. Nat. Commun. 9, 3465 (2018).
54. Brenner, S. The genetics of Caenorhabditis elegans. Genetics 77, 71-94 (1974).
55. Edelstein, A., Amodaj, N., Hoover, K., Vale, R. & Stuurman, N. “Computer Control of Microscopes Using μManager” in Current Protocols in Molecular Biology (eds. Ausubel, F. M. et al.) (John Wiley & Sons, Inc., 2010).
56. Nussbaum-Krammer, C. I., Neto, M. F., Brielmann, R. M., Pedersen, J. S. & Morimoto, R. I. Investigating the spreading and toxicity of prion-like proteins using the metazoan model organism C. elegans. J. Vis. Exp. 8, 52321 (2015).
57. Daval, J.-L. & Nicolas, F. Opposite effects of cyclohexyladenosine and theophylline on hypoxic damage in cultured neurons. Neurosci. Lett. 175, 114-116 (1994).
58. Gasparini, L., Rusconi, L., Xu, H., Soldato, P. D. & Ongini, E. Modulation of β-amyloid metabolism by non-steroidal anti-inflammatory drugs in neuronal cell cultures. J. Neurochem. 88, 337-348 (2004).
59. Boland, B. et al. Autophagy induction and autophagosome clearance in neurons: relationship to autophagic pathology in Alzheimer's Disease. J. Neurosci. 28, 6926-6937 (2008).
60. Posadas, I., Santos, P., Blanco, A., Muñoz-Fernández, M. & Ceña, V. Acetaminophen induces apoptosis in rat cortical neurons. PloS One 5, e15360 (2010).
61. Wang, J. et al. Telmisartan ameliorates glutamate-induced neurotoxicity: roles of AT1 receptor blockade and PPARγ activation. Neuropharmacology 79, 249-261 (2014).
62. Pasternak, B. et al. Use of calcium channel blockers and Parkinson's disease. Am. J. Epidemiol. 175, 627-635 (2012).
63. Ascherio, A. et al. Prospective study of caffeine consumption and risk of Parkinson's disease in men and women. Ann. Neurol. 50, 56-63 (2001).
64. Chen, J. F. et al. Neuroprotection by caffeine and A (2A) adenosine receptor inactivation in a model of Parkinson's disease. J. Neurosci. 21, RC143 (2001).
65. McLeary, F. A. et al. Dexamethasone inhibits copper-induced alpha-synuclein aggregation by a metallothionein-dependent mechanism. Neurotox. Res. 33, 229-238 (2018).
66. La Vitola, P. et al. Alpha-synuclein oligomers impair memory through glial cell activation and via Toll-like receptor 2. Brain. Behav. Immun. 69, 591-602 (2018).
67. Sawada, H. et al. Estradiol protects dopaminergic neurons in a MPP+Parkinson's disease model. Neuropharmacology 42, 1056-1064 (2002).
68. Kaizaki, A. et al. Celecoxib reduces brain dopaminergic neuronaldysfunction, and improves sensorimotor behavioral performance in neonatal rats exposed to systemic lipopolysaccharide. J. Neuroinflammation 10, 45 (2013).
69. Khansari, P. S. & Coyne, L. NSAIDs in the treatment and/or prevention of neurological disorders. Inflammopharmacology 20, 159-167 (2012).
70. Du, Y. et al. Minocycline prevents nigrostriatal dopaminergic neurodegeneration in the MPTP model of Parkinson's disease. Proc. Natl. Acad. Sci. U.S.A 98, 14669-14674 (2001).
71. Styczyńska-Soczka, K., Zechini, L. & Zografos, L. Validating the predicted effect of astemizole and ketoconazole using a Drosophila model of Parkinson's disease. Assay Drug Dev. Technol. 15, 106-112 (2017).
72. Koob, A. O. et al. Lovastatin ameliorates alpha-synuclein accumulation and oxidation in transgenic mouse models of alpha-synucleinopathies. Exp. Neurol. 221, 267-274 (2010).
73. Khan, S. et al. Effect of L-ascorbic Acid on the climbing ability and protein levels in the brain of Drosophila model of Parkinson's disease. Int. J. Neurosci. 122, 704-709 (2012).
74. Hirohata, M., Ono, K., Morinaga, A. & Yamada, M. Non-steroidal anti-inflammatory drugs have potent anti-fibrillogenic and fibril-destabilizing effects for alpha-synuclein fibrils in vitro. Neuropharmacology 54, 620-627 (2008).
75. Mittal, S. et al. β2-Adrenoreceptor is a regulator of the α-synuclein gene driving risk of Parkinson's disease. Science 357, 891-898 (2017).
76. Hebron, M. L., Lonskaya, I. & Moussa, C. E.-H. Nilotinib reverses loss of dopamine neurons and improves motor behavior via autophagic degradation of α-synuclein in Parkinson's disease models. Hum. Mol. Genet. 22, 3315-3328 (2013).
77. Emre, M. Treatment of dementia associated with Parkinson's disease. Parkinsonism Relat. Disord. 13, S457-S461 (2007).
78. Rcom-H'cheo-Gauthier, A. N., Meedeniya, A. C. B. & Pountney, D. L. Calcipotriol inhibits α-synuclein aggregation in S H-SY5Y neuroblastoma cells by a Calbindin-D28k-dependent mechanism. J. Neurochem. 141, 263-274 (2017).
79. Yu, H. C., Feng, S. F., Chao, P. L. & Lin, A. M. Y. Anti-inflammatory effects of pioglitazone on iron-induced oxidative injury in the nigrostriatal dopaminergic system. Neuropathol. Appl. Neurobiol. 36, 612-622 (2010).
80. Ulusoy, G. K. et al. Effects of pioglitazone and retinoic acid in a rotenone model of Parkinson's disease. Brain Res. Bull. 85, 380-384 (2011).
81. Kim, C.-H., Leblanc, P. & Kim, K.-S. 4-amino-7-chloroquinoline derivatives for treating Parkinson's disease: implications for drug discovery. Expert Opin. Drug Discov. 11, 337-341 (2016).
82. Grammatopoulos, T. N., Outeiro, T. F., Hyman, B. T. & Standaert, D. G. Angiotensin I I protects against α-synuclein toxicity and reduces protein aggregation in vitro. Biochem. Biophys. Res. Commun. 363, 846-851 (2007).
83. Grammatopoulos, T. N. et al. Angiotensin type 1 receptor antagonist losartan, reduces MPTP-induced degeneration of dopaminergic neurons in substantia nigra. Mol. Neurodegener. 2, 1 (2007).
84. Gagne, J. J. & Power, M. C. Anti-inflammatory drugs and risk of Parkinson disease. Neurology 74, 995-1002 (2010).
85. Locke, C. J., Fox, S. A., Caldwell, G. A. & Caldwell, K. A. Acetaminophen attenuates dopamine neuron degeneration in animal models of Parkinson's disease. Neurosci. Lett. 439, 129-133 (2008).
86. Madeira, J. M. et al. Novel protective properties of auranofin: Inhibition of human astrocyte cytotoxic secretions and direct neuroprotection. Life Sci. 92, 1072-1080 (2013).
87. Neves, K. R. T. et al. Pentoxifylline neuroprotective effects are possibly related to its anti-inflammatory and TNF-alpha inhibitory properties, in the 6-OHDA model of Parkinson's Disease. Parkinson's Disease 2015, 108179 (2015).
88. Davies, P., Moualla, D. & Brown, D. R. Alpha-synuclein is a cellular ferrireductase. PLOS ONE 6, e15814 (2011).
89. Batta, A., Kalra, B S., Khirasaria, R. (2020). Trends in FDA drug approvals over last 2 decades: An observational study. J Family Med Prim Care. 9 (1), 105-114.
90. Carbone, F., Djamshidian, A., Seppi, K., Poewe, W. (2019). Apomorphine for Parkinson's Disease: Efficacy and Safety of Current and New Formulations. CNS Drugs. 33 (9), 905-918.
91. Chen, K S., Menezes, K., Rodgers, J B., O'Hara, D M., Tran, N., Fujisawa, K., Ishikura, S., Khodaei, S., Chau, H., Lacoste, A., Spangler, S., Visanji, N P., Marras, C., Lozano, A M., Culotti, J., Suo, S., Ryu, W S., Kalia, S K., Kalia, L V. (unpublished).
92. Chen, J., Luo, X., Qiu, H., Mackey, V., Sun, L., Ouyang, X. (2018). Drug discovery and drug marketing with the critical roles of modern administration. Am J Transl Res. 10 (12), 4302-4312.
93. Cox, D., Whiten, D R., Brown, J W P., Horrocks, M H., Gil, R S., Dobson, C M., Klenerman, D., van Oijen, A M., Ecroyd, H. (2018). The small heat shock protein Hsp27 binds α-synuclein fibrils, preventing elongation and cytotoxicity. J Biol Chem. 293 (12), 4486-4497.
94. Daturpalli, S., Waudby, C A., Meehan, S., Jackson, S E. (2013). Hsp90 inhibits α-synuclein aggregation by interacting with soluble oligomers. J Mol Biol. 425 (22), 4614-4628.
95. Dorsey, E R., Sherer, T., Okun, M S., Bloem, B R. (2018). The Emerging Evidence of the Parkinson Pandemic. J Parkinsons Dis. 8 (1), 181474.
96. Du, X., Xie, X., Liu, R. (2020). The Role of α-Synuclein Oligomers in Parkinson's Disease. Int J Mol Sci. 21 (22), 8645.
97. Ehrt, C., Brinkjost, T., Koch, O. (2016). Impact of Binding Site Comparisons on Medicinal Chemistry and Rational Molecular Design. J Med Chem. 59 (9), 4121-4151.
98. Ekimova, I V., Plaksina, D V., Pastukhov, Y F., Lapshina, K V., Lazarev, V F., Mikhaylova, E R., Polonik, S G., Pani, B., Margulis, B A., Guzhova, I V., Nudler, E. (2018). New HSF1 inducer as a therapeutic agent in a rodent model of Parkinson's disease. Exp Neurol. 306 (1), 199-208.
99. FDA. (2020). FDA drug summary report 2020. Available: https://www.fda.gov/drugs/new-drugs-fda-cders-new-molecular-entities-and-new-therapeutic-biological-products/novel-drug-approvals-2020.
100. Friesen, E L., De Snoo, M L., Rajendran, L., Kalia, L V. (2017). Chaperone-Based Therapies for Disease Modification in Parkinson's Disease. Parkinsons Dis. 21 (1), 5307.
101. Friesen, E L., Zhang, Y T., Earnshaw, R., DeSnoo, M L., O'Hara, D M., Agapova, V., Chau, H., Ngana, S., Chen, K S., Kalia, L V., Kalia, S K. (2020). BAG5 Promotes Alpha-Synuclein Oligomer Formation and Functionally Interacts With the Autophagy Adaptor Protein p62. Front Cell Dev Biol. 4 (8), 716.
102. Gao, X., Carroni, M., Nussbaum-Krammer, C., Mogk, A., Nillegoda, N B., Szlachcic, A., Guilbride, D L., Saibil, H R., Mayer, M P., Bukau, B. (2015). Human Hsp70 Disaggregase Reverses Parkinson's-Linked α-Synuclein Amyloid Fibrils. Mol Cell. 59 (5), 781-793.
103. Gentile, F., Agrawal, V., Hsing, M., Ton, A T T., Ban, F., Norinder, U., Gleave, M E., Cherkasov, A. (2020). Deep Docking: A Deep Learning Platform for Augmentation of Structure Based Drug Discovery. ACS Cent Sci. 6 (6), 939-949.
104. Giunta, B., Ehrhart, J., Obregon, D F., Lam, L., Le, L., Jin, J., Fernandez, F., Tan, J., Shytle, D. (2011). Antiretroviral medications disrupt microglial phagocytosis of α-amyloid and increase its production by neurons: implications for HIV-associated neurocognitive disorders. Mol Brain. 4 (1), 23.
105. Goedert, M., Spillantini, M G., Tredici, K D., Braak, H. (2013). 100 years of Lewy pathology. Nat Rev Neurol. 9 (1), 13-24.
106. Gold, J., Rowe, D B., Kiernan, M C., Vucic, S., Mathers, S., Eijk, R P A., Nath, A., Montojo, M G., Norato, G., Santamaria, U A., Rogers, M L., Malaspina, A., Lombardi, V., Mehta, P R., Westeneng, H J., Berg, L H., Al-Chalabi, A. (2019). Safety and tolerability of Triumeq in amyotrophic lateral sclerosis: the Lighthouse trial. Amylotroph Lateral Scler Frontotemporal Degener. 20 (7-8), 595-604.
107. Jin, G., Fu, C., Zhao, H., Cui, K., Chang, J., Wong, S T C. (2012). A novel method of transcriptional response analysis to facilitate drug repositioning for cancer therapy. Cancer Res. 72 (1), 33-44.
108. Kalia, S K., Kalia, L V., McLean, P J. (2010). Molecular chaperones as rational drug targets for Parkinson's disease therapeutics. CNS Neurol Disord Drug Targets. 9 (6), 741-753.
109. Kilpatrick, K., Novoa, J A., Hancock, T., Guerriero, C J., Wipf, P., Brodsky, J L., Segatori, L. (2013). Chemical induction of Hsp70 reduces α-synuclein aggregation in neuroglioma cells. ACS Chem Biol. 8 (7), 1460-1468.
110. Klucken, J., Shin, Y., Masliah, E., Hyman, B T., McLean, P J. (2004). Hsp70 Reduces alpha-Synuclein Aggregation and Toxicity. J Biol Chem. 279 (24), 25497-25502.
111. Koprich, J B., Johnston, T H., Reyes, M G., Sun, X., Brotchie, J M. (2010). Expression of human A53 T alpha-synuclein in the rat substantia nigra using a novel AAV1/2 vector produces a rapidly evolving pathology with protein aggregation, dystrophic neurite architecture and nigrostriatal degeneration with potential to model the pathology of Parkinson's disease. Mol Neurodegener. 28 (5), 34.
112. Lackie, R E., Maciejewski, A., Ostapchenko, V G., Marques-Lopez, J., Choy, W Y., Duennwald, M L., Prado, V F., Prado, M A M. (2017). The Hsp70/Hsp90 Chaperone Machinery in Neurodegenerative Diseases. 16 (11), 3389.
113. Letendre, S., Marquie-Beck, J., Capparelli, E., Best, B., Clifford, D., Collier, A C., Gelman, B B., McArthur, J C., McCutchan, J A., Morgello, S., Simpson, D., Grant, I., Ellis, R J. (2008). Validation of the CNS Penetration-Effectiveness Rank for Quantifying Antiretroviral Penetration Into the Central Nervous System. Arch Neurol. 65 (1), 65-70.
114. LeWitt, P A. (2015). Levodopa therapy for Parkinson's disease: Pharmacokinetics and pharmacodynamics. Mov Disord. 30 (1), 64-72.
115. Li, Y., Jung, N Y., Yoo, J C., Kim, Y., Yi, G S. (2018). Acebutolol, a Cardioselective Beta Blocker, Promotes Glucose Uptake in Diabetic Model Cells by Inhibiting JNK-JIP1 Interaction. Biomol Ther (Seoul). 26 (5), 458-463.
116. Ma, J., Gao, J., Wang, J., Xie, A. (2019). Prion-Like Mechanisms in Parkinson's Disease. Front Neurosci. 13 (1), 552.
117. Maclagan, L C., Visanji, N P., Cheng, Y., Tadrous, M., Lacoste, A M B., Kalia, L V., Bronskill, S E., Marras, C. (2020). Identifying drugs with disease-modifying potential in Parkinson's disease using artificial intelligence and pharmacoepidemiology. Pharmacoepidemiol Drug Saf. 29 (8), 864-872.
118. Mak, K K., Pichika, M R. (2018). Artificial intelligence in drug development: present status and future prospects. Drug Discov Today. 24 (3), 773-780.
119. MatchMaker: A Leap Forward in Proteome Screening Beyond Molecular Docking https://static1.squarespace.com/static/5ed55fed9bdbb618fa024532/t/5ee7b153a1aef52e9a8cdcef/159224251 9500/Cyclica ValidationNote MatchMaker Benchmark.pdf
120. Michael G, Sugiyama., Haotian, Cui., Dar'ya S, Redka., Mehran, Karimzadeh., Edurne, Rujas., Hassaan, Maan., Sikander, Hayat., Kyle, Cheung., Rahul, Misra., Joseph B, McPhee., Russell D, Viirre, Andrew, Haller., Roberto J, Botelho., Raffi, Karshafian., Sarah A, Sabatinos., Gregory D, Fairn., Seyed, Ali., Madani, Tonekaboni., Andreas, Windemuth., Jean-Philippe, Julien., Vijay, Shahani., Stephen S, Mackinnon., Bo, Wang., Costin N, Antonescu. (2021). Multiscale interactome analysis coupled with off-target drug predictions reveals drug repurposing candidates for human coronavirus disease. bioRxiv 2021.04.13.439274; doi: https://doi.org/10.1101/2021.04.13.439274
121. Mdanda, S., Ntshangase, S., Singh, S D., Naicker, T., Kruger, H G., Baijnath, S., Govender, T. (2020). Mass spectrometric investigations into the brain delivery of abacavir, stavudine and didanosine in a rodent model. Xenobiotica. 50 (5), 570-579.
122. Moloney, T C., Hyland, R., O'Toole, D., Paucard, A., Kirik, D., O'Doherty, A., Gorman, A M., Dowd, E. (2014). Heat shock protein 70 reduces α-synuclein-induced predegenerative neuronal dystrophy in the α-synuclein viral gene transfer rat model of Parkinson's disease. CNS Neurosci Ther. 20 (1), 50-58.
123. Nillegoda, N B., Bukau, B. (2015). Metazoan Hsp70-based protein disaggregases: emergence and mechanisms. Front Mol Biosci. 2 (1), 57.
124. Nisar, T., Sutherland-Froggio, H., Husar, W. (2019). Antiviral amantadine. Lancet. 18 (12), 1080.
125. Nooka, S., Ghorpade, A. (2017). HIV-1-associated inflammation and antiretroviral therapy regulate astrocyte endoplasmic reticulum stress responses. Cell Death Discov. 4 (3), 17061.
126. O'Hara, D M., Kalia, S K., Kalia, L K. (2018). Emerging disease-modifying strategies targeting α-synuclein for the treatment of Parkinson's disease. Epub. 175 (15), 3080-3089.
127. Palacios, N., Gao, X,. Mccullough, M L., Schwartzschild, M A., Shah, R., Gapstur, S., Ascherio, A. (2012). Caffeine and risk of Parkinson disease in a large cohort of men and women. Mov Disord. 27 (10), 1276-1282.
128. Picillo, M., Lizarraga, K J., Friesen, E L., Chau, H., Zhang, M., Sato, C., Rooke, G., Munhoz, R P., Rogaeva, E., Fraser, P E., Kalia, S K., Kalia, L V. (2018). Parkinsonism due to A53E α-synuclein gene mutation: Clinical, genetic, epigenetic, and biochemical features. Mov Disord. 33 (12), 1950-1955.
129. Pillaiyar, T., Meenakschisundaram, S., Manickam, M., Sankaranarayanan, M. (2020). A medicinal chemistry perspective of drug repositioning: Recent advances and challenges in drug discovery. Eur J Med Chem. 1 (195), 112275.
130. Polymeropoulos, M H., Lavedan, C., Leroy, E., Ide, S E., Dehejia, A., Dutra, A., Pike, B., Root, H., Rubenstein, J., Boyer, R., Stenroos, E S., Chandrasekharappa, S., Athanassiadou, A., Papapetropoulos, T., Johnson, W G., Lazzarini, A M., Duvoisin, R C., Di lorio, G., Golbe, L I., Nussbaum, R L. (1997). Mutation in the alpha-synuclein gene identified in families with Parkinson's disease. Science. 276 (5321), 2045-2047.
131. Postuma, R B., Anang, J., Pelletier, A., Joseph, L., Mascovich, M., Grimes, D., Furtado, S., Munhoz, R P., Appel-Cresswell, S., Moro, A., Borys, A., Hobson, D,. Land, A E. (2017). Caffeine as symptomatic treatment for Parkinson disease (Café-P D): A randomized trial. Neurology. 89 (17), 1795-1803.
132. Quteiro, T F., Putcha, P., Tetzlaff, J E., Spoelgen, R., Koker, M., Carvalho, F., Hyman, B T., McLean, P J. (2008). Formation of toxic oligomeric alpha-synuclein species in living cells. PLoS One. 3 (4), 1867.
133. Redka, Dar'ya S, Mackinnon, Stephen S, Landon, Melissa., Windemuth, Andreas, Kurji, Naheed., Shahani, Vijay (2020): PolypharmDB, a Deep Learning-Based Resource, Quickly Identifies Repurposed Drug Candidates for COVID-19. ChemRxiv. Preprint. https://doi.org/10.26434/chemrxiv.12071271.v1
134. Riedel, M., Goldbaum, O., Schwartz, L., Schmitt, S., Richter-Landsberg., C. (2010). 17-AAG Induces Cytoplasmic α-Synuclein Aggregate Clearance by Induction of Autophagy. PLoS One. 5 (1), 1.
135. Ruesink, H., Reimer, L., Gregersen, E., Moeller, A., Betzer, C., Jensen, P H. (2019). Stabilization of α-synuclein oligomers using formaldehyde. PLoS One. 14 (10), 1371.
136. Saag, M., Sonnerborg, A., Torres, R A., Lancaster, D., Gazzard, B G., Schooley, R T., Romero, C., Kelleher, D., Spreen, W., LaFon, S. (1998). Antiretroviral effect and safety of abacavir alone and in combination with zidovudine in HIV-infected adults. Abacavir Phase 2 Clinical Team. AIDS. 12 (16), 203-209.
137. Sääksjärvi, K., Rissanen, P K., Laaksonen, M A., Reunanen, A., Männistö, S. (2008). Prospective study of coffee consumption and risk of Parkinson's disease. Eur J Clin Nutr. 7 (908), 1062788.
138. Simuni, T., Oakes, D., Biglan, K,. Galpern, W R., Hauser, K., Hodgeman, E., Kayson, A., Lang, E., Surmeier, D J., Vento, C. (2020). Isradipine Versus Placebo in Early Parkinson Disease A Randomized Trial. Ann Intern Med. 172 (9), 591-598.
139. Sun, W., Sanderson, P E., Zheng, W. (2016). Drug combination therapy increases successful drug repositioning. Drug Discov Today. 21 (7), 1189-1195.
140. Szökö, É., Tábi, T., Riederer, R., Vécsei, L., Magyar, K. (2018). Pharmacological aspects of the neuroprotective effects of irreversible MAO-B inhibitors, selegiline and rasagiline, in Parkinson's disease. J Neural Transm (Vienna). 125 (11), 1735-1749.
141. Tao, J., Berthet, A., Citron, R., Stanley, R., Gestwicki, J., Agard, D A., McConlogue, L. (2020). Hsp70 chaperone blocks α-synuclein oligomer formation via a novel engagement mechanism. 5 (1), 699.
142. Thomas, S A., Bye, A., Segal, M B. (2001). Transport characteristics of the anti-human immunodeficiency virus nucleoside analog, abacavir, into brain and cerebrospinal fluid. J Pharmacol Exp Ther. 298 (3), 947-953.
143. WO2020140156A1-Method and system for predicting drug binding using synthetic data-Google Patents. Available at: https://patents.google.com/patent/WO2020140156A1/en. (Accessed: 1st June 2021)
144. Wang, V., Kuo, T T., Huang, E K., Ma, K H., Chou, Y C., Fu, Z Y., Lai, L W., Jung, J., Choi, H I., Choi, D S., Li, Y., Olson, L., Greig, N H., Hoffer, B J., Chen, Y H. (2021). Sustained Release GLP-1 Agonist PT320 Delays Disease Progression in a Mouse Model of Parkinson's Disease. ACS Pharmacol Transl Sci. 16 (4), 858-869.
145. Wouters, O J., McKee, M., Luyten, J. (2020). Estimated Research and Development Investment Needed to Bring a New Medicine to Market, 2009-2018. JAMA. 323 (9), 844-853.
146. Yang, W., Hamilton, J., Kopil, C., Beck, J C., Tanner, C M., Albin, R L., Dorsey, E R., Dahodwala, N., Cintina, I., Hogan, P., Thompson, T. (2020). Current and projected future economic burden of Parkinson's disease in the U.S. NPJ Parkinsons Dis. 9 (6), 1038.
147. Zafar, F., Valappil, R A., Kim, S., Johansen, K K., Chang, A L S., Tetrud, J W., Eis, P S., Hatchwell, E., Langston, J W., Dickson, D W., Schule, B. (2018). Genetic fine-mapping of the lowan SNCA gene triplication in a patient with Parkinson's disease. NPJ Parkinsons Dis. 4 (18), 1038).
148. Zhang, X M., Anwar, S., Kim, S., Brown, J., Comte, I., Cai, H., Cai, N N., Martins, R., Szele, F G. (2019). The A30P α-synuclein mutation decreases subventricular zone proliferation. Hum Mol Genet. 28 (14), 2283-2294.
149. Zhou, H., Gao, M., Skolnick, J. (2015). Comprehensive prediction of drug-protein interactions and side effects for the human proteome. Nature. 5 (1), 11090.
150. Nalls, Mike A et al. “Identification of novel risk loci, causal insights, and heritable risk for Parkinson's disease: a meta-analysis of genome-wide association studies.” The Lancet. Neurology vol. 18,12 (2019): 1091-1102. doi: 10.1016/S1474-4422 (19) 30320-5.
151. Chia, Ruth et al. “Genome sequencing analysis identifies new loci associated with Lewy body dementia and provides insights into its genetic architecture.” Nature genetics vol. 53,3 (2021): 294-303. doi: 10.1038/s41588-021-00785-3.
152. Noyce, Alastair J et al. “Meta-analysis of early nonmotor features and risk factors for Parkinson disease.” Annals of neurology vol. 72,6 (2012): 893-901. doi: 10.1002/ana.23687
153. Bellou, Vanesa et al. “Environmental risk factors and Parkinson's disease: An umbrella review of meta-analyses.” Parkinsonism & related disorders vol. 23 (2016): 1-9. doi: 10.1016/j.parkreldis.2015.12.008
154. Noyce, Alastair J et al. “PREDICT-P D: An online approach to prospectively identify risk indicators of Parkinson's disease.” Movement disorders: official journal of the Movement Disorder Society vol. 32,2 (2017): 219-226. doi: 10.1002/mds.26898.
155. Schlossmacher, Michael G et al. “Modelling idiopathic Parkinson disease as a complex illness can inform incidence rate in healthy adults: the P R EDIGT score.” The European journal of neuroscience vol. 45,1 (2017): 175-191. doi: 10.1111/ejn.13476.
156. Jack, Clifford R Jr et al. “NIA-A A Research Framework: Toward a biological definition of Alzheimer's disease.” Alzheimer's & dementia: the journal of the Alzheimer's Association vol. 14,4 (2018): 535-562. doi: 10.1016/j.jalz.2018.02.018.
157. McKeith, lan G et al. “Research criteria for the diagnosis of prodromal dementia with Lewy bodies.” Neurology vol. 94,17 (2020): 743-755. doi: 10.1212/WNL.0000000000009323.
158. Berg, Daniela et al. “MDS research criteria for prodromal Parkinson's disease.” Movement disorders: official journal of the Movement Disorder Society vol. 30,12 (2015): 1600-11. doi: 10.1002/mds.26431.
159. Postuma, Ronald B et al. “MDS clinical diagnostic criteria for Parkinson's disease.” Movement disorders: official journal of the Movement Disorder Society vol. 30,12 (2015): 1591-601. doi: 10.1002/mds.26424.
160. McKeith, lan G et al. “Diagnosis and management of dementia with Lewy bodies: Fourth consensus report of the DLB Consortium.” Neurology vol. 89,1 (2017): 88-100. doi: 10.1212/WNL.0000000000004058.
161. Gilman, S et al. “Second consensus statement on the diagnosis of multiple system atrophy.” Neurology vol. 71,9 (2008): 670-6. doi: 10.1212/01.wnl.0000324625.00404.15.
162. McKhann, Guy M et al. “The diagnosis of dementia due to Alzheimer's disease: recommendations from the National Institute on Aging-Alzheimer's Association workgroups on diagnostic guidelines for Alzheimer's disease.” Alzheimer's & dementia: the journal of the Alzheimer's Association vol. 7,3 (2011): 263-9. doi: 10.1016/j.jalz.2011.03.005.
163. Koprich J B, Johnston T H, Reyes M G, Sun X, Brotchie J M. Expression of human A53 T alpha-synuclein in the rat substantia nigra using a novel AAV1/2 vector produces a rapidly evolving pathology with protein aggregation, dystrophic neurite architecture and nigrostriatal degeneration with potential to model the pathology of Parkinson's disease. Mol Neurodegener. 2010; 5 (1): 43. https://doi.org/10.1186/1750-1326-5-43.
164. Vaikath N N, Majbour N K, Paleologou K E, Ardah M T, van Dam E, van de Berg W D J, et al. Generation and characterization of novel conformation-specific monoclonal antibodies for α-synuclein pathology. Neurobiol Dis. 2015; 79:81-99. https://doi.org/10.1016/j.nbd.2015.04.009. 29.
165. O'Hara D M, Kapadia M, Ping S, Kalia S K, Kalia L V. Semi-quantitative determination of dopaminergic neuron density in the substantia Nigra of rodent models using automated image analysis. J Vis Exp. 2021; 168 (168): e62062. https://doi.org/10.3791/62062.
166. Gundersen H J, Jensen E B. The efficiency of systematic sampling in stereology and its prediction. J Microsc. 1987;147 (3): 229-63. https://doi. org/10.1111/j.1365-2818.1987.tb02837.x.
167. Bargar C, Wang W, Gunzler S A, LeFevre A, Wang Z, Lerner A J, et al. Streamlined alpha-synuclein R T-QuIC assay for various biospecimens in Parkinson's disease and dementia with Lewy bodies. Acta Neuropathol Commun. 2021; 9 (1): 62. https://doi.org/10.1186/s40478-021-01175-w.

1. A method of treating a subject with an α-synucleinopathy neurodegenerative disorder or a subject with a high risk of developing an α-synucleinopathy neurodegenerative disorder, or for inhibiting α-synuclein aggregation, the method comprising administering one or more therapeutic(s) to the subject or contacting a cell with the one or more therapeutic(s), wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof.

2. The method of claim 1, wherein the one or more therapeutic(s) is or comprises rifabutin, losartan, lamivudine, and/or abacavir.

3. The method of claim 1, wherein the subject is a mammal, preferably human.

4. (canceled)

5. The method of claim 1, wherein the α-synucleinopathy neurodegenerative disorder is selected from a group comprising Parkinson's Disease, dementia with Lewy bodies, multiple system atrophy, and Alzheimer's disease, optionally wherein the subject is administered the one or more therapeutics immediately following a clinical diagnosis of Parkinson's Disease, dementia with Lewy bodies, multiple system atrophy, Alzheimer's disease.

6. The method of claim 1, wherein the one or more therapeutic(s) are rifabutin, losartan, and abacavir.

7.-10. (canceled)

11. The method of claim 1, wherein the method further comprises administering rapamycin.

12. (canceled)

13. The method of claim 1, for treating a subject with a high risk of developing an α-synucleinopathy neurodegenerative disorder, the method comprising administering one or more therapeutic(s) to the subject, wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof, preferably wherein the one or more therapeutic(s) is or comprises rifabutin, losartan, lamivudine, and/or abacavir.

14. (canceled)

15. The method of claim 13, wherein the subject has known genetic risk factor(s) for developing α-synucleinopathy neurodegenerative disorder.

16. (canceled)

17. The method of claim 13, wherein the subject has prodromal Parkinson's Disease, prodromal dementia with Lewy bodies, prodromal multiple system atrophy, and prodromal Alzheimer's disease (including those with idiopathic/isolated REM sleep behaviour disorder).

18. The method of claim 13, wherein the subject is administered the one or more therapeutic(s) prior to clinical diagnosis of an α-synucleinopathy neurodegenerative disorder.

19. The method of claim 13, wherein the subject is a mammal, preferably a human.

20. (canceled)

21. The method of claim 13, wherein the one or more therapeutic(s) is or comprises rifabutin, losartan, and abacavir.

22.-25. (canceled)

26. The method of claim 13, wherein the method further comprises administering rapamycin.

27. The method of claim 1 for inhibiting α-synuclein aggregation, the method comprising contacting the cell with one or more therapeutic(s), wherein the one or more therapeutic(s) is or comprise rifabutin, one or more nucleoside analog reverse transcriptase inhibitor(s), optionally selected from lamivudine, emtricitabine, tenofovir disoproxil funiarate, tenofovir alafenamide, abacavir, zidovudine, didanosine, and/or stavudine, losartan, or a combination thereof.

28. The method of claim 27, wherein the one or more therapeutic(s) is or comprises rifabutin, losartan, lamivudine, and/or abacavir.

29. The method of claim 27, wherein the cell is a mammalian cell, preferably a human cell, optionally a neuron.

30. (canceled)

31. (canceled)

32. The method of claim 27, wherein the cell is in a subject afflicted with or at risk of developing an α-synucleinopathy neurodegenerative disorder.

33. (canceled)

34. The method of claim 32, wherein the subject has prodromal Parkinson's Disease, prodromal dementia with Lewy bodies, prodromal multiple system atrophy, and prodromal Alzheimer's disease (including those with idiopathic/isolated REM sleep behaviour disorder).

35. The method of claim 32, wherein the subject is administered the one or more therapeutic(s) prior to clinical diagnosis of an α-synucleinopathy neurodegenerative disorder, preferably wherein the one or more therapeutic(s) are rifabutin, losartan, and abacavir.

36.-40. (canceled)

41. The method of claim 27, wherein the method further comprises administering rapamycin.

42.-51. (canceled)
